(19) United States
(12) Patent Application Publication
(10) Pub. No.: US 2015/0105272 A1
(43) Pub. Date: Apr. 16, 2015

(54) BIOMOLECULAR EVENTS IN CANCER REVEALED BY ATTRACTOR METAGENES

(71) Applicant: The Trustees of Columbia University in the City of New York, New York, NY (US)

(72) Inventors: Dimitris Anastassiou, Tenafly, NJ (US); Wei Yi Cheng, New York, NY (US)

(73) Assignee: The Trustees of Columbia University in the City of New York, New York, NY (US)

(21) Appl. No.: 14/519,795

(22) Filed: Oct. 21, 2014

Related U.S. Application Data
(63) Continuation of application No. PCT/US2013/037720, filed on Apr. 23, 2013.
(60) Provisional application No. 61/637,187, filed on Apr. 23, 2012.

(51) Int. Cl. G06F 19/24; C40B 30/02; C12Q 1/68

(52) U.S. Cl. CPC G06F 19/24; C12Q 1/6886; C40B 30/02; C12Q 2600/158; C12Q 2600/118; C12Q 2600/154; C12Q 2600/106
USPC 506/2; 506/16; 506/9; 506/8

(57) ABSTRACT

The present invention is directed to compositions and methods for the independent and unconstrained identification of attractor metagenes as surrogates of pure biomolecular events as well as the use of such attractor metagenes in performing medical diagnosis, prognosis, and developing appropriate therapeutic regimes.

### CROSS REFERENCE TO RELATED APPLICATIONS

The present application is a continuation of PCT Application No. PCT/US13/037,720, filed Apr. 23, 2013, which claims priority to U.S. Provisional Application No. 61/637,187, filed on Apr. 23, 2012, the disclosures of which are incorporated by reference in their entirety.

### 1. BACKGROUND OF THE INVENTION

Rich datasets, such as the rich biomolecular datasets publicly available at an increasing rate from sources such as The Cancer Genome Atlas (TCGA), provide unique opportunities for discovery from purely computational analysis. For example, gene expression signatures resulting from analysis of cancer datasets can serve as surrogates of cancer phenotypes. (Nevins, J. R. & Potti, A. Nat Rev Genet 8, 601-609 (2007)). Subtypes in many cancer types (Collisson et al., Nat Med 17, 500-503 (2011); Verhaak et al., Cancer Cell 17, 98-110 (2010); and Cancer Genome Atlas Research, Nature 474, 609-615 (2011)) have been successfully identified by gene expression analysis often using techniques such as nonnegative matrix factorization (Brunet et al. Proc Natl Acad Sci USA 101, 4164-4169 (2004)) combined with consensus clustering. (Monti, et al., Machine Learning 52, 91-118 (2003)).

The main objective addressed by techniques such as nonnegative matrix factorization is to reduce dimensionality by identifying a number of metagenes jointly representing the gene expression dataset as accurately as possible, in lieu of the whole set of individual genes. Each metagene is defined as a positive linear combination of the individual genes, so that its expression level is an accordingly weighted average of the expression levels of the individual genes. The identity of each resulting metagene is influenced by the presence of other metagenes within the objective of overall dimensionality reduction achieved by joint optimization.

In contrast, if the aim is not dimensionality reduction or classification into subtypes, but instead the independent and unconstrained identification of metagenes as surrogates of pure biomolecular events, then a different algorithm should be devised. This approach is devoid of cross-interference and has the advantage of increasing the chance of precisely identifying the few particular genes that are at the core of the underlying biological mechanism as those that have the highest weights in the corresponding metagene, thus shedding more light on that mechanism. The present invention relates to such a novel approach, including in the context of applications involving data sets other than those related to gene expression, as well as the metagenes identified thereby, and compositions & methods employing such metagenes.

### 2. SUMMARY OF THE INVENTION

In certain embodiments, the present invention is directed to compositions and methods for identifying an attractor from a data set, comprising: evaluating the data set, wherein the data set comprises information concerning a plurality of objects characterized by particular feature vectors and wherein the evaluation identifies, using a computer processor, an association between individual members of the plurality of objects; and selecting, from the plurality of objects, a set of two or more objects maximally associated with a composite version of the same set of objects, and thereby identifying an attractor from the data set.

In certain embodiments, the present invention is directed to compositions and methods for identifying an attractor metagene from a gene data set, comprising: evaluating the gene data set, wherein the gene data set comprises information from a plurality of genes and wherein the evaluation identifies, using a computer processor, an association between individual members of the plurality of genes; and selecting, from the plurality of genes, a set of two or more genes maximally associated with a composite version of the same set of genes, and thereby identifying an attractor metagene from the gene data set.

In certain embodiments of such methods, the composite version of the gene set comprising the attractor metagene is a weighted average of the individual genes in which the weights are proportional to the associations of the corresponding individual genes with the metagene. In certain embodiments of such methods, said evaluation consists of an iterative process in which each iteration modifies a metagene defined as a weighted average of individual genes such that the weights become increasingly proportional to the associations of the corresponding individual genes with the metagene. In certain embodiments of such methods, the evaluation consists of an iterative process in which each iteration modifies a metagene comprising individual genes such that the individual genes are increasingly associated with a composite version of the same set of genes. In certain embodiments of such methods, the gene data set comprises expression levels for each of the plurality of genes. In certain embodiments of such methods, the gene data set comprises methylation values for each of the plurality of genes.

In certain embodiments, the present invention is directed to a system for identifying an attractor metagene from a gene data set, comprising: at least one processor and a computer readable medium coupled to the at least one processor, the computer readable medium having stored thereon instructions which when executed cause the processor to: evaluate the gene data set, wherein the gene data set comprises information from a plurality of genes and wherein the evaluation identifies, using the computer processor, an association between individual members of plurality of genes; and selecting, from the plurality of genes, a set of two or more genes maximally associated with a composite version of the same set of genes, and thereby identifying an attractor metagene from the gene data set.

In certain embodiments of such systems, the composite version of the gene set comprising the attractor metagene is a weighted average of the individual genes in which the weights are proportional to the associations of the corresponding individual genes with the metagene. In certain embodiments of such systems, the evaluation consists of an iterative process in which each iteration modifies a metagene comprising individual genes such that the individual genes are increasingly associated with a composite version of the same set of genes. In certain of such embodiments, the gene data set comprises expression levels for each of the plurality of genes. In certain of such embodiments, the gene data set comprises methylation values for each of the plurality of genes.

In certain embodiments, the present invention is directed to a kit for detecting the presence of an attractor metagene comprising measuring means for one or more biomarker selected from the group consisting of the genes associated with an attractor metagene of FIG. 1A-1, 1A-2, 1B-1, 1B-2, 1B-3, 1B-4, 1B-5, 1B-6, Table 2, Table 3, or Table 4 where the measuring means is, for each biomarker to be measured, a pair of oligonucleotide primers capable of hybridizing the biomarker.

In certain embodiments, the present invention is directed to a kit for detecting the presence of a mesenchymal attractor metagene comprising measuring means for one or more biomarker selected from the group consisting of the genes associated with the attractor metagene of Table 2, where the measuring means is, for each biomarker to be measured, a pair of oligonucleotide primers capable of hybridizing the biomarker.

In certain embodiments, the present invention is directed to a kit for detecting the presence of a mitotic CIN attractor metagene comprising measuring means for one or more biomarker selected from the group consisting of the genes associated with the attractor metagene of Table 3, where the measuring means is, for each biomarker to be measured, a pair of oligonucleotide primers capable of hybridizing the biomarker.

In certain embodiments, the present invention is directed to a kit for detecting the presence of a lymphocyte-specific attractor metagene comprising measuring means for one or more biomarker selected from the group consisting of the genes associated with the lymphocyte-specific attractor metagene of FIG. 1A-1, 1A-2, 1B-1, 1B-2, 1B-3, 1B-4, 1B-5 and 1B-6, where the measuring means is, for each biomarker to be measured, a pair of oligonucleotide primers capable of hybridizing the biomarker.

In certain embodiments, the present invention is directed to a kit for detecting the presence of a lymphocyte-specific attractor metagene comprising measuring means for one or more biomarker selected from the group consisting of the genes associated with the lymphocyte-specific attractor metagene of Table 4, where the measuring means is, for each biomarker to be measured, a pair of oligonucleotide primers capable of hybridizing the biomarker.

In certain embodiments, the present invention is directed to a kit for detecting the presence of a Chr8q24.3 amplicon attractor metagene comprising measuring means for one or more biomarker selected from the group consisting of the genes associated with the Chr8q24.3 amplicon attractor metagene of FIG. 1A-1, 1A-2, 1B-1, 1B-2, 1B-3, 1B-4, 1B-5 and 1B-6, where the measuring means is, for each biomarker to be measured, a pair of oligonucleotide primers capable of hybridizing the biomarker.

In certain embodiments, the present invention is directed to a kit for detecting the presence of a Chr17q12 HER2 amplicon attractor metagene comprising measuring means for one or more biomarker selected from the group consisting of the genes associated with a Chr17q12 HER2 amplicon attractor metagene of FIG. 1A-1, 1A-2, 1B-1, 1B-2, 1B-3, 1B-4, 1B-5 and 1B-6, where the measuring means is, for each biomarker to be measured, a pair of oligonucleotide primers capable of hybridizing the biomarker.

In certain of the foregoing embodiments relating to kits, the present invention is also directed to kits that further comprise a control sample.

In certain embodiments, the present invention is directed to a method of treatment wherein a patient sample is assayed for the presence of one or more biomarker selected from the group consisting of the genes associated with an attractor metagene of FIG. 1A-1, 1A-2, 1B-1, 1B-2, 1B-3, 1B-4, 1B-5, 1B-6, Table 2, Table 3, or Table 4 and wherein, if the biomarker associated with the attractor metagene is present, thereafter adjusting the treatment accordingly.

In certain embodiments, the present invention is directed to a method of treatment wherein a patient sample is assayed for the presence of one or more biomarker selected from the group consisting of the genes associated with the mesenchymal attractor metagene of Table 2 and wherein, if the biomarker associated with the attractor metagene is present, thereafter adjusting the treatment accordingly.

In certain embodiments, the present invention is directed to a method of treatment wherein a patient sample is assayed for the presence of one or more biomarker selected from the group consisting of the genes associated with the mitotic CIN attractor metagene of Table 3, and wherein, if the biomarker associated with the attractor metagene is present, thereafter adjusting the treatment accordingly.

In certain embodiments, the present invention is directed to a method of treatment wherein a patient sample is assayed for the presence of one or more biomarker selected from the group consisting of the genes associated with the lymphocyte-specific attractor metagene of FIG. 1A-1, 1A-2, 1B-1, 1B-2, 1B-3, 1B-4, 1B-5 and 1B-6, and wherein, if the biomarker associated with the attractor metagene is present, thereafter adjusting the treatment accordingly.

In certain embodiments, the present invention is directed to a method of treatment wherein a patient sample is assayed for the presence of one or more biomarker selected from the group consisting of the genes associated with the lymphocyte-specific attractor metagene of Table 4 and wherein, if the biomarker associated with the attractor metagene is present, thereafter adjusting the treatment accordingly.

In certain embodiments, the present invention is directed to a method of treatment wherein a patient sample is assayed for the presence of one or more biomarker selected from the group consisting of the genes associated with the Chr8q24.3 amplicon attractor metagene of FIG. 1A-1, 1A-2, 1B-1, 1B-2, 1B-3, 1B-4, 1B-5 and 1B-6, and wherein, if the biomarker associated with the attractor metagene is present, thereafter adjusting the treatment accordingly.

In certain embodiments, the present invention is directed to a method of treatment wherein a patient sample is assayed for the presence of one or more biomarker selected from the group consisting of the genes associated with the Chr17q12 HER2 amplicon attractor metagene of FIG. 1A-1, 1A-2, 1B-1, 1B-2, 1B-3, 1B-4, 1B-5 and 1B-6, and wherein, if the biomarker associated with the attractor metagene is present, thereafter adjusting the treatment accordingly.

In certain embodiments, the present invention provides for methods of performing a prognosis of a subject identified as having cancer, such as, but not limited to, methods comprising performance of a diagnostic method as set forth herein (e.g., obtaining a sample from the subject and determining whether an attractor metagene can be detected in the sample) and then, if an attractor metagene is detected in a sample of the subject, predicting the likely outcome (i.e., performing a prognosis) of the cancer, e.g., the likely survival duration. In certain embodiments, the prognosis will be based on the presence of one or more attractor metagenes. In certain embodiments, the prognosis will be based on the presence of one or more attractor metagenes and one or more additional factors, such as clinical and molecular features (e.g., the number of cancer-positive lymph nodes, age at diagnosis, and expression levels of particular genes exhibiting protective activity).

### 3. BRIEF DESCRIPTION OF THE FIGURES

FIGS. 1A-1, 1A-2, 1B-1, 1B-2, 1B-3, 1B-4, 1B-5 and 1B-6 includes a summarization of a series of multi-cancer attractors. FIGS. 1A-1 and 1A-2 contains the general attractors, and FIGS. 1B-1, 1B-2, 1B-3, 1B-4, 1B-5 and 1B-6 contains attractors of genes located close to the other in the genome, which in certain, but not all, cases represent amplicons.

FIGS. 2A-B depicts analysis of the Mitotic CIN attractor metagene. (A and B) Kaplan-Meier cumulative survival curves of breast cancer patients over a 15-year period on the basis of the mitotic CIN attractor metagene expression—represented by the CIN feature—in the (A) METABRIC and (B) OsloVal data sets. The patients were divided into equal-sized “high” and “low” CIN-expressing subgroups according to their ranking with respect to expression values of the CIN feature. High expression of the mitotic CIN attractor metagene was associated with poorer survival in both data sets. P values derived using the log-rank test in the two data sets were less than 2×10⁻¹⁶ and 0.041, respectively.

FIGS. 3A-C depicts analysis of the LYM attractor metagene. (A and B) Kaplan-Meier cumulative survival curves of ER-negative breast cancer patients over a 15-year period on the basis of LYM attractor metagene expression—represented by the LYM feature—in the (A) METABRIC and (B) OsloVal data sets. The ER-negative breast cancer patients were divided into equal-sized high and low LYM expressing subgroups according to their ranking with respect to expression values of the LYM feature. High expression of the LYM attractor metagene was associated with improved survival in both data sets. P values derived using the log-rank test in the two data sets were 0.0024 and 0.0223, respectively. (C) Kaplan-Meier cumulative survival curves of ER-positive breast cancer patients with more than four positive lymph nodes over a 15-year period on the basis of LYM attractor metagene expression—represented by the LYM feature—in the METABRIC data set. ER-positive breast cancer patients with more than four positive lymph nodes were divided into equal-sized high and lowLYM-expressing subgroups according to their ranking with respect to expression values of the LYM feature. In contrast to (A), high expression of the LYM attractor metagene was associated with poorer survival in this patient subset. The P value derived using the log-rank test was 0.0278. There were only 19 corresponding samples in the OsloVal data set, insufficient for validation of this reversal relative to (B).

FIGS. 4A-D depicts analysis of the FGD3-SUSD3 metagene. (A) A scatter plot of the expression of SUSD3 versus FGD3 in the METABRIC data set shows a high variance in the expression of both genes at high expression levels. On the other hand, low expression of one strongly suggests low expression of the other in breast tumors. (B) ER-negative breast tumors tended not to express the FGD3-SUSD3 metagene, whereas ER-positive breast tumors may or may not express the FGD3-SUSD3 metagene. (C and D) Kaplan-Meier cumulative survival curves of breast cancer patients over a 15-year period on the basis of FGD3-SUSD3 metagene expression in the (C) METABRIC and (D) OsloVal data sets. Patients were divided into equal-sized high and low subgroups according to their ranking with respect to FGD3-SUSD3 metagene expression values. Low levels of FGD3-SUSD3 metagene expression were associated with poor survival in both data sets. P values derived using the log-rank test in the two data sets were less than 2×10⁻¹⁶ and 0.0028, respectively.

FIG. 5 depicts the results achieved with the final ensemble model. Shown are Kaplan-Meier cumulative survival curves of breast cancer patients over a 15-year period on the basis of the predictions made by the final ensemble model in the OsloVal data set. The patients were divided into equal-sized poor and good predicted survival subgroups according to the ranking assigned by the final model, which was trained on the METABRIC data set. The P value derived using the log-rank test was less than 2×10⁻¹⁶.

FIGS. 6A-C depict a schematic of model development for the model described in Example 2. Shown are block diagrams that describe the development stages for the final ensemble prognostic model. Building a prognostic model involves derivation of relevant features, training submodels and making predictions, and combining predictions from each submodel. The model derived the attractor metagenes using gene expression data, combined them with the clinical information through Cox regression, GBM, and KNN techniques, and eventually blended each submodel's prediction.

FIGS. 7A-C depict the corresponding attractors for the CIN metagene in the PANCAN12 data. Highlighted (light shading for top 10, dark shading for remaining 90) are the genes from Table 1 that appear in the PANCAN12 data.

FIGS. 8A-C depict the corresponding attractors for the MES metagene in the PANCAN12 data. Highlighted (light shading for top 10, dark shading for remaining 90) are the genes from Table 2 that appear in the PANCAN12 data.

FIGS. 9A-C depict the corresponding attractors for the LYM metagene in the PANCAN12 data. Highlighted (light shading for top 10, dark shading for remaining 90) are the genes from Table 3 that appear in the PANCAN12 data.

FIGS. 10A-F depict scatter plots of the top three genes of the CIN attractor metagene in the context of the various cancer types present in the PANCAN12 data sets publicly available from the Cancer Genome Atlas.

FIGS. 11A-F depict scatter plots of the top three genes of the LYM attractor metagene in the context of the various cancer types present in the PANCAN12 data sets publicly available from the Cancer Genome Atlas.

FIGS. 12A-F depict scatter plots of the top three genes of the MES attractor metagene in the context of the various cancer types present in the PANCAN12 data sets publicly available from the Cancer Genome Atlas.

FIGS. 13A-F depict scatter plots of the top three genes of a previously disclosed early mesenchymal transition attractor metagene in the context of the various cancer types present in the PANCAN12 data sets publicly available from the Cancer Genome Atlas.

FIGS. 14A-F depict scatter plots of the top three genes of the chr8q24.3 attractor metagene (excluding MYC) in the context of the various cancer types present in the PANCAN12 data sets publicly available from the Cancer Genome Atlas.

### 4. DETAILED DESCRIPTION OF THE INVENTION

The present invention is directed to compositions and methods for the independent and unconstrained identification of attractors out of rich datasets. For example, given a rich dataset represented by a gene expression matrix, such surrogate metagenes can be naturally identified as stable and precise attractors using a simple iterative approach. The identification processes of the present invention can be totally unsupervised, as the processes need not make use of any phenotypic association. Once identified, however, a metagene attractor is likely to be found associated with a phenotype. This approach is devoid of cross-interference and has the advantage of increasing the chance of precisely identifying the few particular genes that are at the core of the underlying biological mechanism as those that have the highest weights in the corresponding metagene, thus shedding more light on that mechanism. While the identification of attractor metagenes is employed throughout the instant application, it is appreciated that virtually any rich dataset can be analyzed in this fashion to identify relevant attractors—whether it be gene expression data, physiological data, or even commercial data.

The present invention is directed, in part, to compositions and methods for the independent and unconstrained identification of metagenes as surrogates of pure biomolecular events. Given a rich dataset represented by a gene expression matrix, such surrogate metagenes can be naturally identified as stable and precise attractors using a simple iterative approach. The identification processes of the present invention can be totally unsupervised, as the processes need not make use of any phenotypic association. Once identified, however, a metagene attractor is likely to be found associated with a phenotype. This approach is devoid of cross-interference and has the advantage of increasing the chance of precisely identifying the few particular genes that are at the core of the underlying biological mechanism as those that have the highest weights in the corresponding metagene, thus shedding more light on that mechanism.

In certain embodiments, attractor metagenes have been identified as present in nearly identical form in multiple cancer types. This provides an additional opportunity to combine the powers of a large number of rich datasets to focus, at an even sharper level, on the core genes of the underlying mechanism. For example, this methodology can precisely point to the causal (driver) oncogenes within amplicons to be among very few candidate genes. This can be done from rich gene expression data, which already exist in abundance, without the requirement of generating and/or using sequencing data.

For clarity and not by way of limitation, this detailed description is divided into the following sub-portions:
4.1. Identification of Attractor Metagenes;
4.2. Mesenchymal Transition Attractor;
4.3. Mitotic CIN Attractor;
4.4. A Lymphocyte-Specific Attractor;
4.5. Chr8q24.3 Amplicon Attractor;
4.6. Chr17q12 HER2 Amplicon Attractor; and
4.7. Diagnosis & Treatment Employing Attractor Metagenes

4.1. Identification of Attractor Metagenes

4.1.1. Introduction to Attractor Metagenes

While the instant application is directed, in part, to the identification and use of “attractor metagenes,” the techniques described herein for identifying attractors find significantly broader use than solely in connection with gene expression data. For example, but not by way of limitation, the algorithms described herein can be used for identifying attractors present in virtually any rich dataset, whether it relates to gene expression data, physiological activity (e.g., neuronal activity), or even commercial data (e.g., purchasing patterns or the use of social media). Thus, while the identification of genes will be employed as one example of the algorithms disclosed herein, the scope of the instant application is not so limited and can be implemented to identify objects characterized by any type of feature vectors.

Given a nonnegative measure J(Gᵢ, Gⱼ) of pairwise association between genes Gᵢ and Gⱼ, an attractor metagene can be defined as

$$M = {\sum }_{i} {w}_{i} {G}_{i}$$

to be a linear combination of the individual genes with weights wᵢ=J(Gᵢ, M). The association measure J is assumed to have minimum possible value 0 and maximum possible value 1, so the same is true for the weights. It is also assumed to be scale-invariant, therefore it is not necessary for the weights to be normalized so that they add to 1, and the metagenes can still be thought of as expressing a normalized weighted average of the expression levels of the individual genes.

According to this definition, the genes with the highest weights in an attractor metagene will have the highest association with the metagene (and, by implication, they will tend to be highly associated among themselves) and so they will often represent a biomolecular event reflected by the co-expression of these top genes. This can happen, e.g., when a biological mechanism is activated, or when a copy number variation (CNV), such as an amplicon, is present, in some of the samples included in the expression matrix.

As used herein, the tem “attractor metagene,” means a signature of coexpressed genes and the phrase “top genes” refers to the genes with the highest weights in a particular attractor metagene. However, in certain embodiments, the definition of an attractor metagene can readily be generalized to include features other than gene expression, such as, but not limited to, methylation values. In certain embodiments, the term attractor can be used in datasets of any objects (not necessarily genes) characterized by any type of feature vectors.

The computational problem of identifying attractor metagenes given an expression matrix can be addressed heuristically using a simple iterative process: Starting from a particular seed (or “attractee”) metagene M, a new metagene is defined in which the new weights are wᵢ=J(Gᵢ, M). The same process is then repeated in the next iteration resulting in a new set of weights, and so forth. Given a sufficient number of iterations, such a process will converge to a limited number of stable attractors. Each attractor is defined by a precise set of weights, which are reached with high accuracy, and, in certain embodiments, within 10 or 20 iterations.

This algorithmic behavior with convergence properties occurs due to the fact that if a metagene contains some co-expressed genes with high weights, then the next iteration will naturally “attract” even more genes with the same properties, and so forth, until the process will eventually converge to a metagene representing a potential underlying biological event reflected by this co-expression. Therefore, in certain embodiments, this methodology provides an unsupervised algorithm of identifying biomolecular events from rich biological data. Furthermore, in certain embodiments, the set of the few genes with the highest weight can represent the “heart” (core) of the biomolecular event. In support of this concept, the association of any of the top-ranked individual genes with the attractor metagene is consistently and significantly higher than the pairwise association between any of these genes, suggesting that, in certain embodiments, the set of these top genes are synergistically associated, comprising a proxy representing a biomolecular event in a better way than each of the individual genes would. In certain embodiments, these proxy attractor metagenes can then be used within the context of Bayesian methods to identify regulatory interactions in a more straightforward manner than having to jointly identify clusters of co-expressed genes and regulatory interactions.

Indeed, in certain instances, particular aspects of attractors identified using the techniques described herein have been previously identified in various contexts, often intermingled with additional genes that may be unrelated or weakly related with the actual underlying mechanism. The techniques described herein, however, allow for recognition of certain attractors as multi-cancer biomolecular events and their composition is “purified” as a result of the attractor convergence to represent the core of the mechanism. Therefore the top genes of the attractors will be most appropriate to be used as biomarkers or for improved understanding of the underlying biology and for identifying potential therapeutic targets. For example, certain aspects related to the mitotic CIN attractor descried herein have been previously described generally (Whitfield et al., Nat Rev Cancer 6, 99-106 (2006)) as “proliferation” or “cell-cycle related” markers, while the actual attractor, identified for the first time herein, points much more sharply to particular elements in the kinetochore structure.

In certain embodiments, a reasonable implementation of an “exhaustive” search will include only consider the seed metagenes in which one selected “attractee” gene is assigned a weight of 1 and all the other genes are assigned a weight of 0. The metagene resulting from the next iteration will then assign high weights to all genes highly associated with the originally selected gene, referred to as the “attractee gene.” In this way all attractors representing biomolecular events characterized by coordinately co-expressed genes will be identified when these genes are used as attractees. A computational implementation of an algorithm associated to such an embodiment is described in the Examples section, below. In certain embodiments, a dual method can be used to identify attractor “metasamples” as representatives of subtypes, and in certain embodiments such metasamples can be combined with the attractor metagenes in various ways to achieve biclustering.

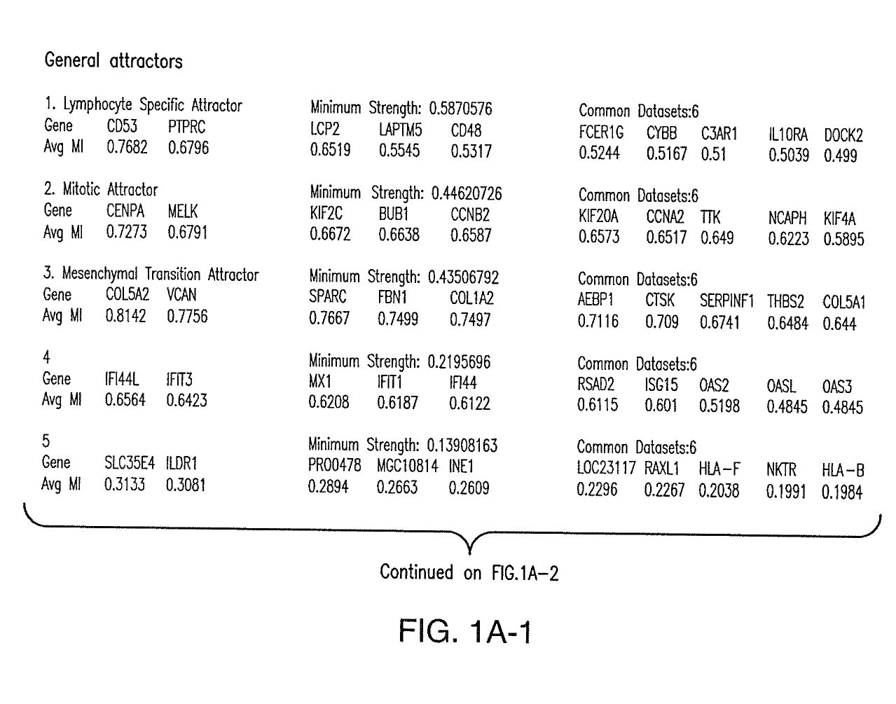

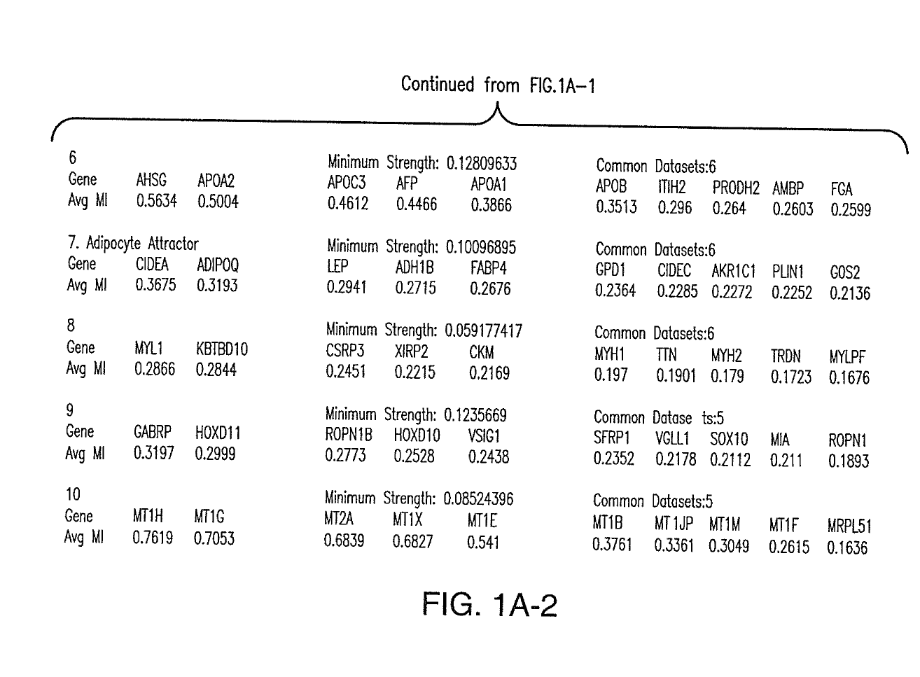

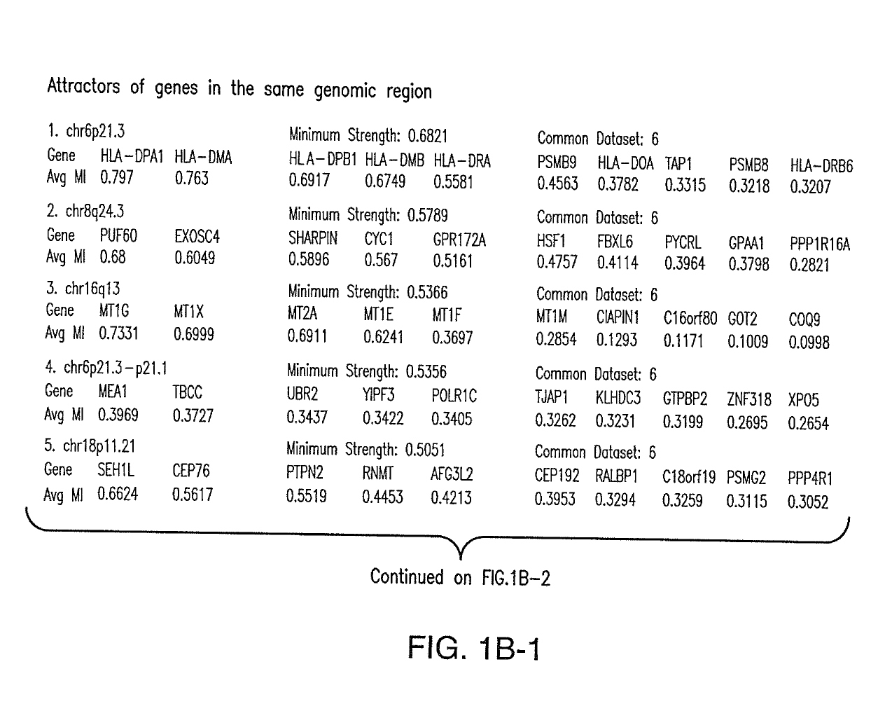

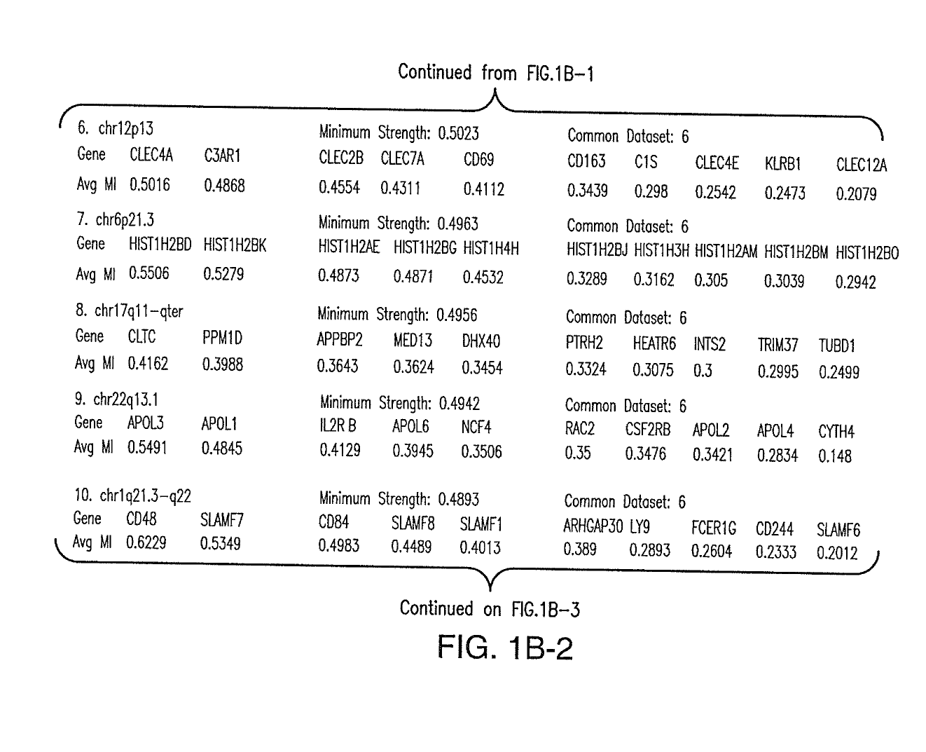

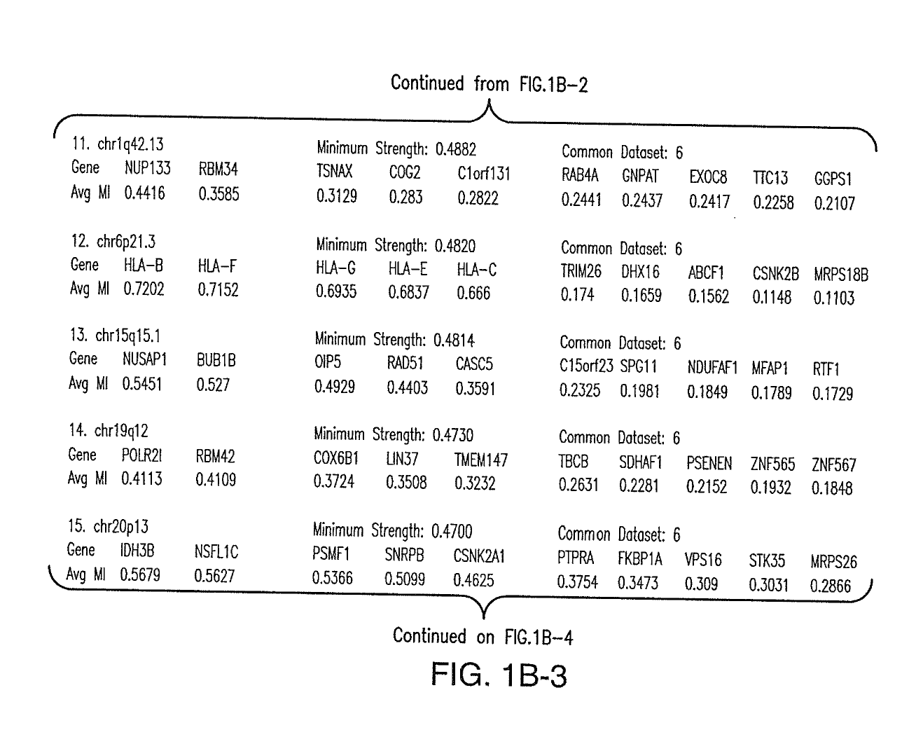

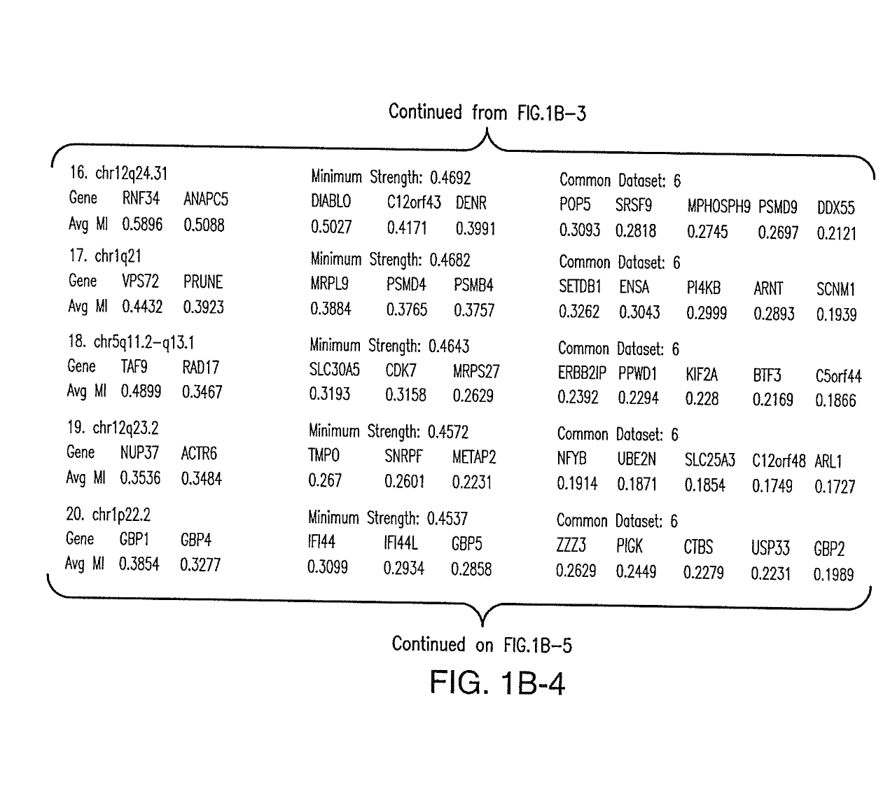

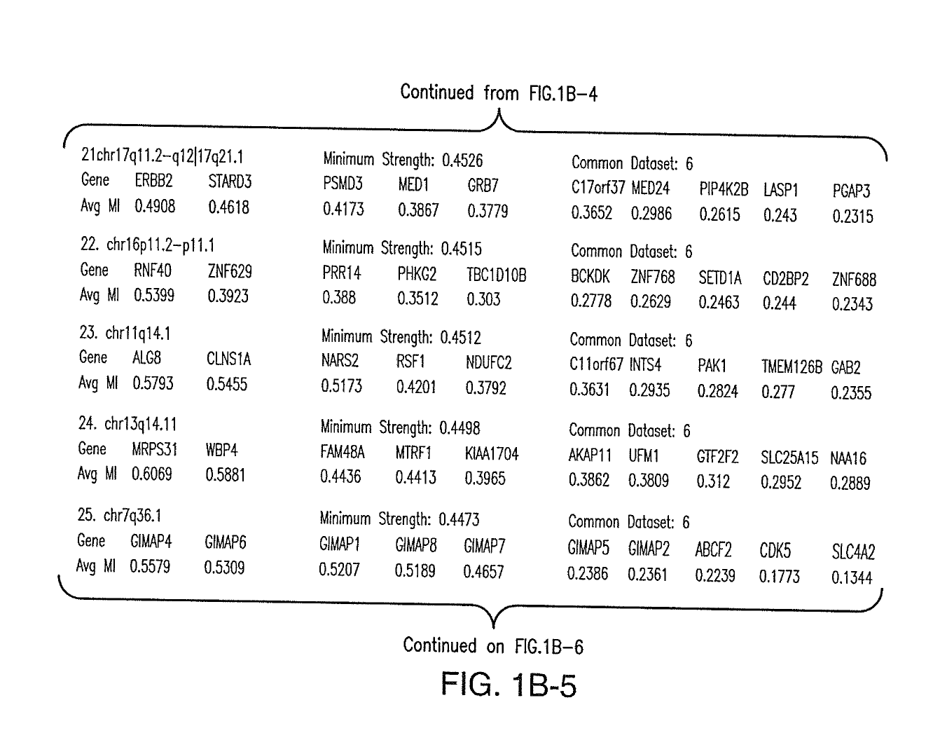

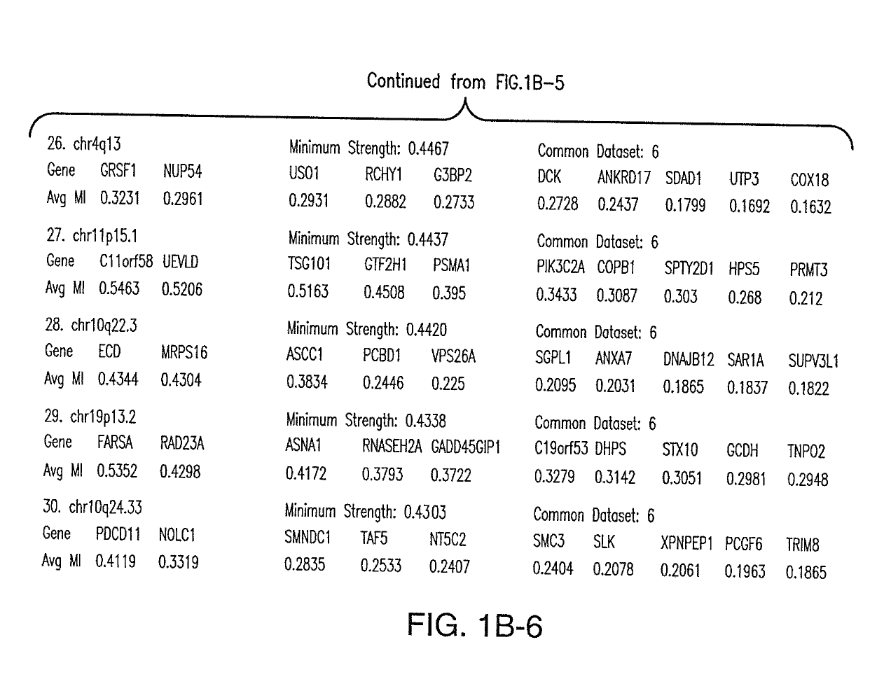

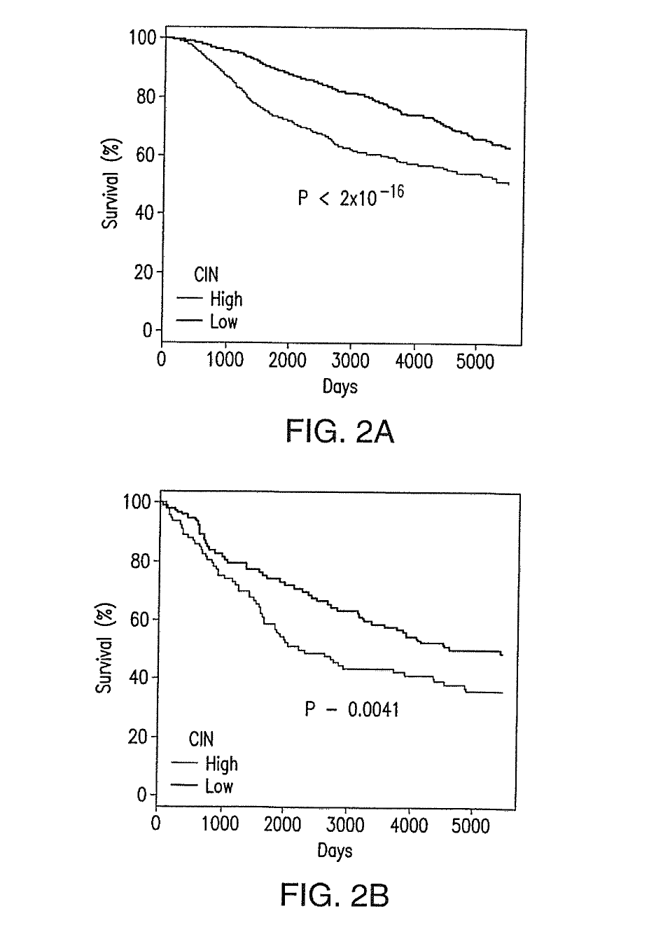

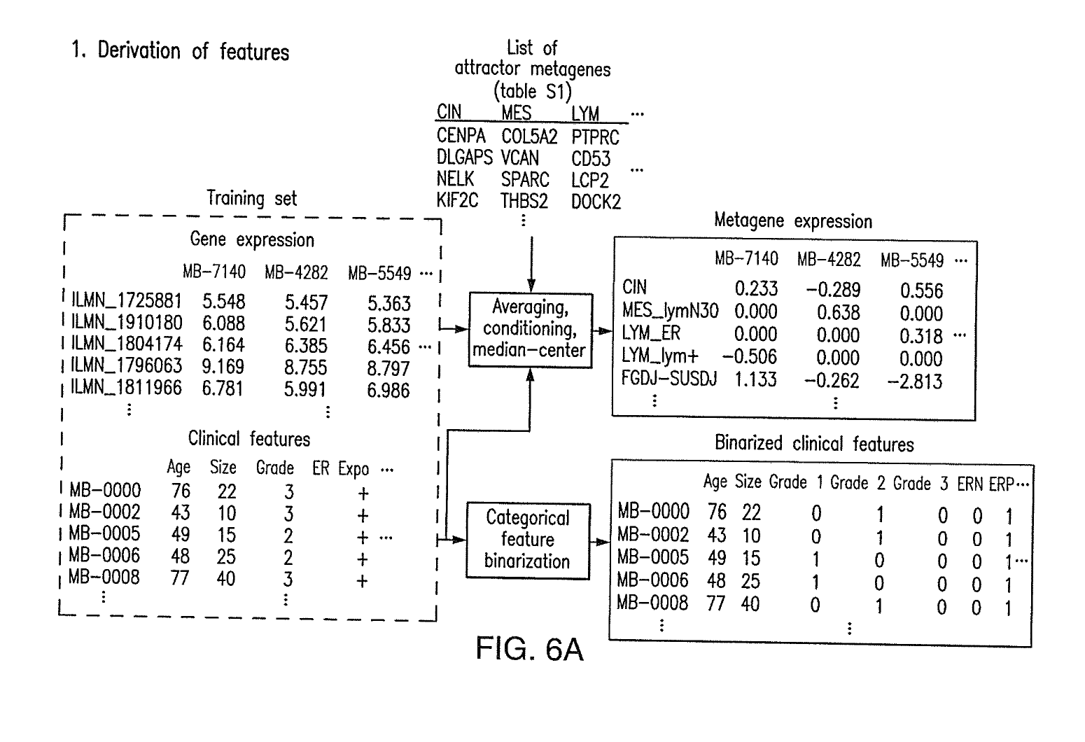

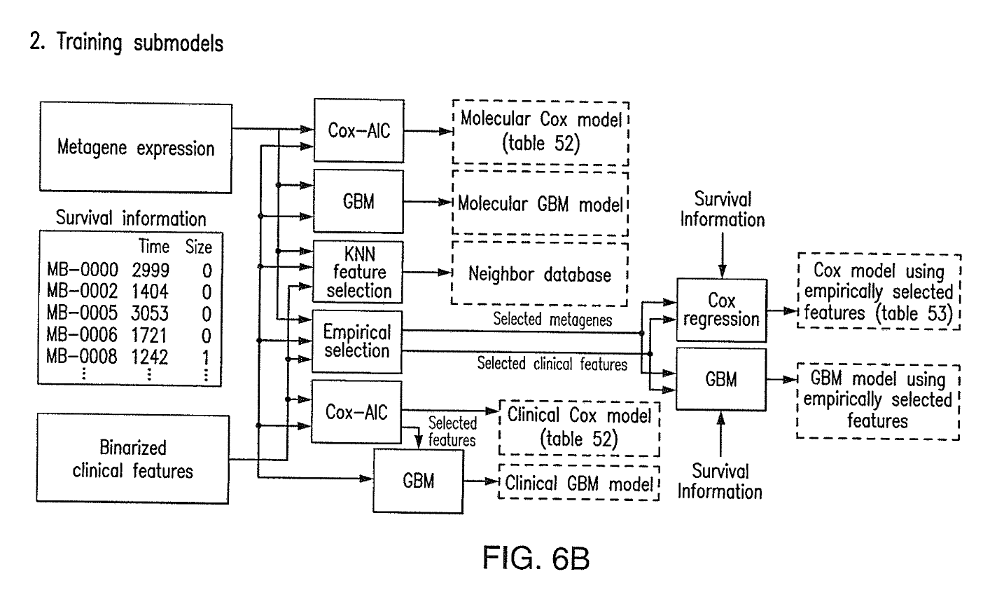

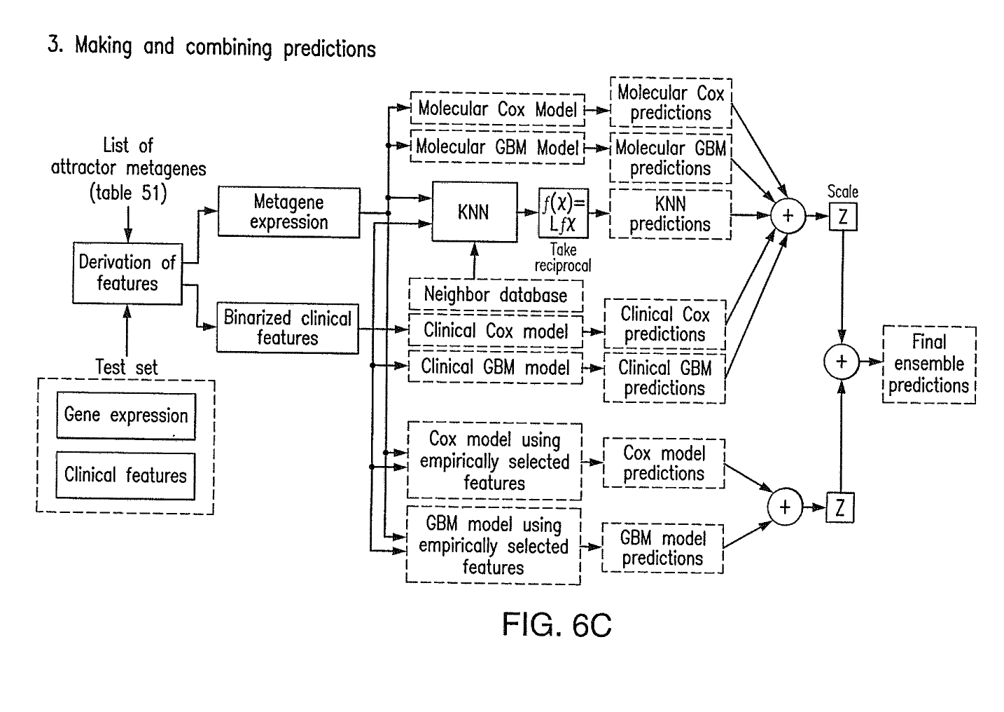

As outlined in the Example 1, below, six datasets, two from ovarian cancer, two from breast cancer and two from colon cancer (Table 1) were initially analyzed in indentifying the attractor metagenes disclosed herein. In each case, general (see FIGS. 1A-1 and 1A-2) and amplicon (see FIGS. 1B-1, 1B-2, 1B-3, 1B-4, 1B-5 and 1B-6) attractors were found separately and most of these attractors appear in similar forms in all six datasets. The criteria used for merging and ranking the attractors in each case are set forth in detail in the following sections. As outlined in Examples 2 and 3, below, the attractors can be identified in additional data sets, validating their diagnostic and prognostic value.

TABLE 1
Lists of datasets used to derive attractors

| Dataset | Sample Size | Platform |
|---|---|---|
| Breast Wang | 286 | Affymetrix HG-U133A |
| (GSE2034) | | |
| Breast TCGA | 536 | Agilent 244K Custom Gene |
| | | Expression G4502A-07-03 |
| Colon Jorrison | 290 | Affymetrix HG-U133Plus 2.0 |
| (GSE14333) | | |
| Colon TCGA | 154 | Agilent 244K Custom Gene |
| | | Expression G4502A-07-3 |
| Ovarian Tothill | 285 | Affymetrix HG-U133Plus 2.0 |
| (GSE9891) | | |
| Ovarian TCGA | 584 | Affymetrix HG-U133A |

4.1.2. General Attractor Finding Algorithm

As noted above, while the instant application describes the identification of attractors in the context of gene information, the general attractor finding algorithm described herein can be applied to virtually any rich data set, regardless of the particular nature of the data. Accordingly, while the instant application will describe the use of algorithms in the particular context of identifying attractor metagenes, it is understood that alternative attractors, depending the nature of the data set, can be identified. Thus, in the context of identifying attractor metagenes the association measure J(Gᵢ, Gⱼ) between genes (which in other contexts would represent the association measure between two alternative factors) is selected to be a power function with exponent a of a normalized estimated information theoretic measure of the mutual information I(Gᵢ, Gⱼ) with minimum value 0 and maximum value 1, as a proper compromise between performance and complexity (although more sophisticated related association measures can also be used). (Cover, T. M. & Thomas, J. A. Elements of information theory, Edn. 2nd. Wiley-Interscience, Hoboken, N.J.; (2006); and Reshef et al., Science 334, 1518-1524 (2011)). In other words, J(Gᵢ, Gⱼ)=I^{a}(Gᵢ, Gⱼ), in which the exponent a can be any nonnegative number. As described in Examples section, each iteration of the algorithm will define a new metagene in which the weight wᵢ for gene Gᵢ will be equal to wᵢ=J(Gᵢ, M), where M is the immediately preceding metagene. The process is repeated until the magnitude of the difference between two consecutive weight vectors is less than a threshold, which can be selected, in certain embodiments, to be equal to 10⁻⁷.

In certain embodiments, algorithms useful in the context of the present invention can be described in simple MATLAB computer language as follows:

when given a gene expression matrix “E” of size ngenes×nsamples, where “ngenes” is the number of genes and “nsamples” is the number of samples. The single-row vector “weights” has size ngenes and contains the corresponding weights of a metagene. In each iteration, the metagene, being the weighted average of the expression values of the individual genes, is modified according to the following MATLAB code, in which “association” is an association measure function between two genes defined by their expression values:

```
for j=1:nsamples
  metagene (j) = weights*E(:,j);
end
for i=1:ngenes
  weights(i)= association(E(i), metagene)
end.
```

Alternatively, the attractor finding algorithm can identify unweighted “attractor gene sets” of size “attractorsize,” which can be fixed or adaptively varying. In that case, if the indices of the rows of the member genes are defined by a vector named “members,” then the metagene will be the simple average of the member genes. Each iteration leads to a new gene set consisting of the new set of top-ranked genes in terms of their association with the previous metagene. Therefore, in each iteration, the metagene will be modified as follows:

metagene = mean(E(members,:),1);
for i=1:ngenes
vect(i)=association(E(i), metagene);
end
[Y I] = sort(vect,‘descend’);
members = I(1:attractorsize).

In certain embodiments, the result of the instant process is tunable in terms of a parameter of “sharpness” of the attractor. This sharpness is based on a nonlinear function “f” of a known original association function “I” like the mutual information or the Pearson coefficient. Thus, in certain embodiments, the final “association function J” used to fit the definition of attractor can be f(I)=I^{a}, where the range of the continuously varying exponent “a” can be from zero to infinity. In certain non-limiting embodiments, “a” will be a large number, e.g., 10-10 or a very small number, e.g., from about 0.5 to 10⁻¹⁰. At one extreme, if “a” is very large then each of the seeds will create its own single-gene attractor because all other genes will always have near-zero weights. In such embodiments, the total number of attractors will be equal to the number of genes. At the other extreme, if “a” is zero then all weights will remain equal to each other, thus representing the average of all genes, so there will only be one attractor. The higher the value of “a,” the “sharper” (more focused on its top gene) each attractor will be and the higher the overall number of attractors will be. As the value of “a” is gradually decreased, the attractor from a particular seed will transform itself, and in certain embodiments in a discontinuous manner, thus providing insight into potential related biological mechanisms.

In certain embodiments, an appropriate choice of “a” (in the sense of revealing single biomolecular events of co-expressed genes) for general attractors is around is from about 0.5 to about 10, in certain embodiments from 1 to about 6, and in certain embodiments a is about 5. In embodiments where a is about 5, there will typically be approximately 50 to 150 resulting attractors, each resulting from numerous attractee genes, depending on the number of genes and the cancer type. (An alternative to the power function can be a sigmoid function with varying steepness, but the consistency of the resulting attractors can, in certain embodiments, be decreased as compared to other techniques).

In certain embodiments, an attractor metagene can also be interpreted as a set of co-expressed genes containing a number among the top genes of the attractor. In such cases, one can define the size of such set so that the set contains only the genes that are significantly associated with the attractor. One empirical such criterion would be to include the genes whose z-score of their mutual information with the attractor exceeds a large threshold, such as, but not limited to, exceeding a z-score of 20.

Identified attractors can be ranked in various ways. In certain embodiments, the “strength of an attractor” will be defined as the mutual information between the n^{th} top gene of the attractor and the attractor metagene itself. Indeed, if this measure is high, this implies that at least the top n genes of the attractor are strongly co-expressed. In certain embodiments, n=50 can be selected as a reasonable choice, not too large, but sufficiently so to represent a real complex biological phenomenon of co-expression of at least 50 genes. For amplicons, n=5 is sufficient to ensure that the oncogenes are included in the co-expression).

4.1.3. Amplicon Finding Algorithm

In certain embodiments, the top genes of an attractor are in a similar chromosomal location. In such cases, the biomolecular event that they represent can be the presence of a particular copy number variation, such as, but not limited to, the presence of an amplicon.

To identify amplicons, the same algorithm can be used as described above, but for each seed gene the set of candidate attractor genes is restricted to only include those in the local genomic neighborhood of the gene, and the exponent “a” is optimized so that the strength of the attractor is maximized. Specifically, the genes in each chromosome are sorted in terms of their genomic location and only the genes within a window of size 51 are considered, i.e., with 25 genes on each side of the seed gene. The choice of the exponent “a” can be optimized for each seed, by allowing “a” to range from 1.0 to 6.0 with step size of 0.5 and selecting the attractor with the highest strength.

Because the set of allowed genes is different for each seed, the attractors will be different from each other, but “neighboring” attractors will usually be very similar to each other. Therefore, following exhaustive attractor finding by considering each seed gene in a chromosome, a filtering algorithm is applied to only select the highest-strength attractor in each local genomic region, as follows: For each attractor, all the genes are first ranked in terms of their mutual information with the corresponding attractor metagene and the range of the attractor is defined to be the chromosomal range of its top 15 genes. If there is any other attractor with overlapping range and higher strength, then the former attractor will be filtered out. This filtering is done in parallel so elimination of attractors occurs simultaneously. The remaining “winning” attractors are assumed to correspond to real amplicons. Of course, the co-expression of the genes in such attractors will still occasionally be due to other co-regulation biological mechanisms, as in the local region of a major histocompatibility complex. They may also be due to copy number deletions, rather than amplifications. In all cases, however, the resulting locally focused attractors will still be useful.

4.1.4. Mutual Information Estimation

Assuming that the continuous expression levels of two genes G₁ and G₂ are governed by a joint probability density p₁₂ with corresponding marginals p₁ and p₂ and using simplified notation, the mutual information [symbol] I(G [symbol] ₁, G₂) is defined as the expected value of log(p₁₂/p₁p₂). It is a non-negative quantity representing the information that each one of the variables provides about the other. The pairwise mutual information has successfully been used as a general measure of the correlation between two random variables. Mutual information can be computed with a spline-based estimator using six bins in each dimension. (Daub et al., BMC Bioinformatics 5, 118 (2004)). This method divides the observation space into equally spaced bins and blurs the boundaries between the bins with spline basis functions using third-order B-splines. The estimated mutual information can be further normalized by dividing by the maximum of the estimated [symbol] I(G [symbol] ₁, G₂) and [symbol] I(G [symbol] ₁, G₂), so the maximum possible value of [symbol] I(G [symbol] ₁, G₂) is 1.

4.1.5. Pre-Processing Gene Expression Datasets

Among the list of datasets in Table 1, Level 3 data can be used when directly available, and imputed missing values using a k-nearest-neighbor algorithm with k=10, as implemented in Troyanskaya et al., Bioinformatics 17, 520-525 (2001). The other datasets on the Affymetrix platform can be normalized using the RMA algorithm as implemented in the affy package in Gautier et al., Bioinfoimatics 20, 307-315 (2004).

To avoid biasing attractor convergence with multiple correlated probe sets of the same gene, the probe set-level expression values can be summarized into the gene-level expression values by taking the mean of the expression values of probe sets for the same genes. The annotations for the probe sets given in the jetset package can be used as well. (Li et al., BMC Bioinformatics 12, 474 (2011).

To investigate the associations between the attractor metagene expression and the tumor stage and grade, the following, non-limiting, annotated gene expression datasets can be used. For stage association: Breast (GSE3893), TCGA Ovarian, Colon (GSE14333). For grade association: Breast (GSE3494), TCGA Ovarian, Bladder (GSE13507). In certain embodiments, for Breast GSE3494, only the samples profiled by U133A arrays are used. In certain embodiments, for Breast GSE3893, two platforms can be combined by taking the intersections of the probes in the U133A and the U133Plus 2.0 arrays. In certain embodiments, such as, but not limited to those datasets profiled by Affymetrix platforms, all the datasets can be normalized using the RMA algorithm. In certain embodiments, for Bladder GSE13507, normalization is provided in the dataset itself

4.1.6. Clustering Attractors in Multiple Datasets

In certain embodiments, after applying the attractor finding algorithms in the six datasets of Table 1, any attractors that resulted from less than three attractee (seed) genes can be filtered out. To identify common attractors in different datasets, the genes in each attractor can be first ranked according to their mutual information with the attractor metagene, selecting the top 50 genes as its representative “attractor gene set.” Hierarchical clustering can then be performed on the attractor gene sets. The clustering algorithm iteratively defines “attractor clusters,” each of which only contains attractors from distinct datasets (i.e. its maximum size is six). The “similarity score” between two attractor clusters can be defined to be the number of overlapping genes among all possible pairs of attractor gene sets between two attractor clusters. If two attractor clusters both contain gene sets from the same datasets, then they are not clustered together. Starting from the two attractor gene sets with highest similarity score, the process can proceed until there is no attractor cluster pair that can be further clustered together. An exemplary result of such clustering is given in FIGS. 1A-1 and 1A-2.

4.1.7. Clustering Amplicon Attractors in Multiple Datasets

All amplicon attractors can be ranked in each dataset according to their strength and the same clustering algorithm as described above can be used, except that attractor gene sets have size 15 and the similarity score is set to 1 if two attractors are overlapping and 0 if their ranges are exclusive. An exemplary result of such clustering of amplicons is given in FIGS. 18-1, 1B-2, 1B-3, 1B-4, 1B-5 and 1B-6.

4.2. Mesenchymal Transition Attractor Metagene

This attractor contains mostly epithelial-mesenchymal transition (EMT)-associated genes. Table 2 provides a listing of top 100 genes based on their average mutual information with their corresponding attractor metagenes.

This is a stage-associated attractor, in which the signature is significantly present only when a particular level of invasive stage, specific to each cancer type, has been reached. This phenomenon is observed, in three cancer datasets from different types (breast, ovarian and colon) that were annotated with clinical staging information, by providing a listing of differentially expressed genes, ranked by fold change, when ductal carcinoma in situ (DCIS) progresses to invasive ductal carcinoma; colon cancer progresses to stage II; and ovarian cancer progresses to stage III. In all three cases, the attractor is highly enriched among the top genes. Specifically, among the top 100 differentially expressed genes, the number of attractor genes included Table 2 were 55 in breast cancer, 45 in ovarian cancer and 31 in colon cancer. The corresponding Fisher's exact test P values are 3×10⁻¹⁰⁹, 9×10⁻⁸³ and 5×10⁻⁶², respectively.

This attractor has been previously identified with remarkable accuracy as representing a particular kind of mesenchymal transition of cancer cells present in all types of solid cancers tested leading to a published list of top 64 genes. (Kim et al., BMC Med Genomics 3, 51 (2010); and Anastassiou et al., BMC Cancer 11, 529 (2011)). Indeed 56 of these top 64 genes appear in Table 2 (P<10⁻¹²⁷), and all top 24 genes of Table 2 are among the 64. Most of the genes of the signature were found to be expressed by the cancer cells themselves, and not by the surrounding stroma, at least in a neuroblastoma xenograft model. (Anastassiou et al., BMC Cancer 11, 529 (2011)). The signature is found to be associated with prolonged time to recurrence in glioblastoma. (Cheng et al., PLoS One 7, e34705 (2012). Related versions of the same signature were previously found to be associated with resistance to neoadjuvant therapy in breast cancer. (Farmer et al., Nat Med 15, 68-74 (2009)). These results are consistent with the finding that EMT induces cancer cells to acquire stem cell properties. (Mani et al., Cell 133, 704-715 (2008)). It has been hypothesized that EMT is a key mechanism for cancer cell invasiveness and motility. (Hay, Acta Anat (Basel) 154, 8-20 (1995); Thiery, Nat Rev Cancer 2, 442-454 (2002); and Kalluri et al., J Clin Invest 119, 1420-1428 (2009)). The attractor, however, appears to represent a more general phenomenon of transdifferentiation present even in nonepithelial cancers such as neuroblastoma, glioblastoma and Ewing's sarcoma.

Although similar signatures are often labeled as “stromal,” because they contain many stromal markers such as α-SMA and fibroblast activation protein, the fact that most of the genes of the signature were expressed by xenografted cancer cells (Anastassiou et al., BMC Cancer 11, 529 (2011)), and not by mouse stromal cells, suggests that this particular attractor of coordinately expressed genes represents cancer cells having undergone a mesenchymal transition. The signature may indicate a non-fibroblastic transition, as occurs in glioblastoma, in which case collagen COL11A1 is not co-expressed with the other genes of the attractor. It is believed that a full fibroblastic transition of the cancer cells occurs when cancer cells encounter adipocytes (Anastassiou et al., BMC Cancer 11, 529 (2011)), in which case they may well assume the duties of cancer associated fibroblasts (CAFs) in some tumors. Hanahan et al., Cell 144, 646-674 (2011)). In that case, the best proxy of the signature (Kim et al., BMC Med Genomics 3, 51 (2010)) is COL11A1 and the strongly co-expressed genes THBS2 and INHBA. Indeed, the 64 genes of the previously identified signature were found from multi-cancer analysis (Kim et al., BMC Med Genomics 3, 51 (2010)) as the genes whose expression is consistently most associated with that of COL11A1.

The only EMT-inducing transcription factor found upregulated in the xenograft model (Anastassiou et al., BMC Cancer 11, 529 (2011)) is SNAI2 (Slug), and it is also the one most associated with the signature in publicly available datasets. The microRNAs found to be most highly associated with this attractor are miR 214, miR 199a, and miR-199b. Interestingly, miR-214 and miR-199a were found to be jointly regulated by another EMT-inducing transcription factor, TWIST1¹⁷. (Yin et al., Oncogene 29, 3545-3553 (2010)).

TABLE 2
Top 100 genes of the mesenchymal transition attractor
based on six datasets.

| | Gene | |
|---|---|---|
| Rank | Symbol | Avg MI |
| 1 | COL5A2 | 0.814 |
| 2 | VCAN | 0.775 |
| 3 | SPARC | 0.766 |
| 4 | THBS2 | 0.758 |
| 5 | FBN1 | 0.749 |
| 6 | COL1A2 | 0.749 |
| 7 | COL5A1 | 0.747 |
| 8 | FAP | 0.734 |
| 9 | AEBP1 | 0.711 |
| 10 | CTSK | 0.709 |
| 11 | COL3A1 | 0.688 |
| 12 | COL1A1 | 0.683 |
| 13 | SERPINF1 | 0.674 |
| 14 | COL6A3 | 0.669 |
| 15 | CDH11 | 0.663 |
| 16 | GLT8D2 | 0.658 |
| 17 | LUM | 0.654 |
| 18 | MMP2 | 0.654 |
| 19 | DCN | 0.650 |
| 20 | CCDC80 | 0.637 |
| 21 | POSTN | 0.631 |
| 22 | CTHRC1 | 0.616 |
| 23 | ADAM12 | 0.613 |
| 24 | COL6A2 | 0.608 |
| 25 | MSRB3 | 0.608 |
| 26 | OLFML2B | 0.607 |
| 27 | INHBA | 0.600 |
| 28 | FSTL1 | 0.600 |
| 29 | SFRP2 | 0.596 |
| 30 | SNAI2 | 0.577 |
| 31 | CRISPLD2 | 0.574 |
| 32 | PCOLCE | 0.571 |
| 33 | PDGFRB | 0.567 |
| 34 | BGN | 0.565 |
| 35 | COL12A1 | 0.560 |
| 36 | ANGPTL2 | 0.555 |
| 37 | COPZ2 | 0.553 |
| 38 | CMTM3 | 0.549 |
| 39 | ASPN | 0.547 |
| 40 | FN1 | 0.545 |
| 41 | CNRIP1 | 0.540 |
| 42 | FNDC1 | 0.538 |
| 43 | LRRC15 | 0.533 |
| 44 | COL11A1 | 0.529 |
| 45 | ANTXR1 | 0.528 |
| 46 | RAB31 | 0.527 |
| 47 | FRMD6 | 0.524 |
| 48 | TSHZ3 | 0.520 |
| 49 | THY1 | 0.519 |
| 50 | NNMT | 0.519 |
| 51 | SULF1 | 0.505 |
| 52 | LOXL1 | 0.502 |
| 53 | PRRX1 | 0.502 |
| 54 | PPAPDC1A | 0.499 |
| 55 | COL10A1 | 0.498 |
| 56 | ITGA11 | 0.495 |
| 57 | NTM | 0.494 |
| 58 | MXRA8 | 0.494 |
| 59 | FIBIN | 0.493 |
| 60 | WISP1 | 0.483 |
| 61 | RCN3 | 0.483 |
| 62 | TNFAIP6 | 0.481 |
| 63 | ECM2 | 0.480 |
| 64 | HTRA1 | 0.480 |
| 65 | EFEMP2 | 0.478 |
| 66 | MXRA5 | 0.474 |
| 67 | ACTA2 | 0.472 |
| 68 | LOX | 0.470 |
| 69 | ITGBL1 | 0.466 |
| 70 | PMP22 | 0.465 |
| 71 | P4HA3 | 0.464 |
| 72 | PTRF | 0.463 |
| 73 | CALD1 | 0.460 |
| 74 | HEG1 | 0.458 |
| 75 | NEXN | 0.455 |
| 76 | NID2 | 0.455 |
| 77 | TAGLN | 0.455 |
| 78 | FAM26E | 0.452 |
| 79 | ZNF521 | 0.452 |
| 80 | SFRP4 | 0.451 |
| 81 | PALLD | 0.450 |
| 82 | OLFML1 | 0.447 |
| 83 | FILIP1L | 0.447 |
| 84 | TIMP3 | 0.445 |
| 85 | SPON2 | 0.443 |
| 86 | SPOCK1 | 0.443 |
| 87 | COL8A2 | 0.441 |
| 88 | GPC6 | 0.438 |
| 89 | PDPN | 0.437 |
| 90 | GFPT2 | 0.436 |
| 91 | LHFP | 0.436 |
| 92 | GREM1 | 0.436 |
| 93 | TGFB1I1 | 0.435 |
| 94 | C1S | 0.433 |
| 95 | EDNRA | 0.432 |
| 96 | GAS1 | 0.431 |
| 97 | NOX4 | 0.431 |
| 98 | FBLN2 | 0.428 |
| 99 | TCF4 | 0.428 |
| 100 | NUAK1 | 0.427 |

4.3. Mitotic CIN Attractor Metagene

This attractor contains mostly kinetochore-associated genes. Table 3 provides a listing of top 100 genes based on their average mutual information with their corresponding attractor metagenes.

Contrary to the stage associated mesenchymal transition attractor, this is a grade associated attractor, in which the signature is significantly present only when an intermediate level of tumor grade is reached. This phenomenon can be observed, in three cancer datasets from different types (breast, ovarian and bladder) that were annotated with tumor grade information, by providing a listing of differentially expressed genes, ranked by fold change, when grade G2 is reached. In all three cases, the attractor is highly enriched among the top genes. Specifically, among the top 100 differentially expressed genes, the number of attractor genes included Table 3 were 41 in breast cancer, 36 in ovarian cancer and 26 in colon cancer. The corresponding Fisher's exact test P values are 7×10⁻⁷³, 4×10⁻⁶¹ and 5×10⁻⁴⁷, respectively. Consistently, a similar “gene expression grade index” signature was previously found differentially expressed between histologic grade 3 and histologic grade 1 breast cancer samples. (Sotiriou et al., Journal of the National Cancer Institute 98, 262-272 (2006)). Furthermore, that same signature was found capable of reclassifying patients with histologic grade 2 tumors into two groups with high versus low risks of recurrence. (Sotiriou et al., Journal of the National Cancer Institute 98, 262-272 (2006)).

This attractor is associated with chromosomal instability (CIN), as evidenced from the fact that another similar gene set comprising a “signature of chromosomal instability” (Carter et al., Nat Genet 38, 1043-1048 (2006)) was previously derived from multiple cancer datasets purely by identifying the genes that are most correlated with a measure of aneuploidy in tumor samples. This led to a 70-gene signature referred to as “CIN70.” Indeed 34 of these 70 genes appear in Table 3 (P<10⁻⁶¹). However, several top genes of the attractor, such as CENPA, KIF2C, BUB1 and CCNA2 are not present in the CIN70 list. Mitotic CIN is increasingly recognized as a widespread multi-cancer phenomenon. (Schvartzman, J. M., Sotillo, R. & Benezra, R. Mitotic chromosomal instability and cancer: mouse modelling of the human disease. Nat Rev Cancer 10, 102-115 (2010)).

The attractor is characterized by overexpression of kinetochore-associated genes, which are known (Yuen et al., Current Opinion in Cell Biology 17, 576-582 (2005)) to induce chromosomal instability (CIN) for reasons that are not clear. Overexpression of several of the genes of the attractor, such as the top gene CENPA (Amato et al., Mol Cancer 8, 119 (2009)), as well as MAD2L1 (Sotillo et al., Nature 464, 436-440 (2010)) and TPX2 (Heidebrecht et al., Mol Cancer Res 1, 271-279 (2003)), has also been independently previously found associated with CIN. Included in the mitotic CIN attractor are key components of mitotic checkpoint signaling (Orr-Weaver et al., Nature 392, 223-224 (1998)), such as BUB1B, MAD2L1 (aka MAD2), CDC20, and TTK (MSP1). It was recently found (Birkbak et al., Cancer Res 71, 3447-3452 (2011)) that the CIN70 signature is most strongly associated with poor outcome at intermediate, rather than extreme levels. This is consistent with the concept that, while cancer cells are intolerant of extreme instability, moderate mitotic chromosomal instability may provide a proliferative advantage.

Among transcription factors, MYBL2 (aka B-Myb) and FOXM1 were found to be strongly associated with the attractor. They are already known to be sequentially recruited to promote late cell cycle gene expression to prepare for mitosis. (Sadasivam et al., Genes & development 26, 474-489 (2012)).

Inactivation of the retinoblastoma (RB) tumor suppressor promotes CIN (Manning et al., Nat Rev Cancer 12, 220-226 (2012)) and the expression of the attractor signature. Indeed, a similar expression of a “proliferation gene cluster” (Rosty et al., Oncogene 24, 7094-7104 (2005)) was found strongly associated with the human papillomavirus E7 oncogene, which abrogates RB protein function and activates E2F-regulated genes. Consistently, many among the genes of the attractor correspond to E2F pathway genes controlling cell division or proliferation. Among the E2F transcription factors, E2F8 and E2F7 were found to be most strongly associated with the attractor.

TABLE 3
Top 100 genes of the mitotic chromosomal instability attractor
based on six datasets.

| | Gene | |
|---|---|---|
| Rank | Symbol | Avg MI |
| 1 | CENPA | 0.720 |
| 2 | DLGAP5 | 0.693 |
| 3 | MELK | 0.684 |
| 4 | BUB1 | 0.674 |
| 5 | KIF2C | 0.660 |
| 6 | KIF20A | 0.658 |
| 7 | KIF4A | 0.656 |
| 8 | CCNA2 | 0.654 |
| 9 | CCNB2 | 0.652 |
| 10 | NCAPG | 0.649 |
| 11 | TTK | 0.642 |
| 12 | CEP55 | 0.638 |
| 13 | CCNB1 | 0.632 |
| 14 | CDK1 | 0.629 |
| 15 | HJURP | 0.626 |
| 16 | CDC20 | 0.624 |
| 17 | CDCA5 | 0.615 |
| 18 | NCAPH | 0.615 |
| 19 | BUB1B | 0.609 |
| 20 | KIF23 | 0.592 |
| 21 | KIF11 | 0.591 |
| 22 | BIRC5 | 0.589 |
| 23 | NUF2 | 0.587 |
| 24 | TPX2 | 0.586 |
| 25 | AURKB | 0.582 |
| 26 | RACGAP1 | 0.580 |
| 27 | NUSAP1 | 0.580 |
| 28 | ASPM | 0.579 |
| 29 | MCM10 | 0.579 |
| 30 | PRC1 | 0.576 |
| 31 | DEPDC1B | 0.572 |
| 32 | UBE2C | 0.569 |
| 33 | UBE2T | 0.567 |
| 34 | NEK2 | 0.566 |
| 35 | FOXM1 | 0.565 |
| 36 | NDC80 | 0.556 |
| 37 | CDCA3 | 0.556 |
| 38 | FAM54A | 0.553 |
| 39 | ANLN | 0.551 |
| 40 | KIF15 | 0.548 |
| 41 | STIL | 0.547 |
| 42 | EXO1 | 0.542 |
| 43 | AURKA | 0.540 |
| 44 | PTTG1 | 0.539 |
| 45 | OIP5 | 0.539 |
| 46 | RRM2 | 0.539 |
| 47 | DEPDC1 | 0.539 |
| 48 | CDKN3 | 0.538 |
| 49 | KIF14 | 0.537 |
| 50 | SPC25 | 0.534 |
| 51 | CDCA8 | 0.532 |
| 52 | CDC45 | 0.528 |
| 53 | KIF18A | 0.524 |
| 54 | HMMR | 0.506 |
| 55 | TOP2A | 0.505 |
| 56 | CENPF | 0.503 |
| 57 | ZWINT | 0.503 |
| 58 | PLK1 | 0.501 |
| 59 | RAD51AP1 | 0.501 |
| 60 | FAM83D | 0.498 |
| 61 | E2F8 | 0.497 |
| 62 | CENPE | 0.497 |
| 63 | MKI67 | 0.492 |
| 64 | CENPN | 0.491 |
| 65 | MAD2L1 | 0.489 |
| 66 | CHEK1 | 0.486 |
| 67 | GTSE1 | 0.477 |
| 68 | RAD51 | 0.475 |
| 69 | SGOL2 | 0.474 |
| 70 | PARPBP | 0.469 |
| 71 | TRIP13 | 0.467 |
| 72 | SHCBP1 | 0.465 |
| 73 | DTL | 0.465 |
| 74 | CENPL | 0.462 |
| 75 | FEN1 | 0.461 |
| 76 | FANCI | 0.461 |
| 77 | FBXO5 | 0.459 |
| 78 | ECT2 | 0.457 |
| 79 | MND1 | 0.456 |
| 80 | CDC25C | 0.456 |
| 81 | PBK | 0.456 |
| 82 | KPNA2 | 0.452 |
| 83 | RAD54L | 0.452 |
| 84 | ESPL1 | 0.447 |
| 85 | CDCA2 | 0.446 |
| 86 | FAM64A | 0.440 |
| 87 | CENPK | 0.436 |
| 88 | MYBL2 | 0.435 |
| 89 | SPAG5 | 0.434 |
| 90 | EZH2 | 0.431 |
| 91 | SMC4 | 0.430 |
| 92 | TACC3 | 0.428 |
| 93 | C11orf82 | 0.427 |
| 94 | MASTL | 0.426 |
| 95 | ASF1B | 0.426 |
| 96 | PTTG3P | 0.425 |
| 97 | CENPW | 0.424 |
| 98 | ORC1 | 0.424 |
| 99 | HELLS | 0.422 |
| 100 | TK1 | 0.421 |

4.4. A Lymphocyte-Specific Attractor Metagene

A strong lymphocyte-specific attractor was identified as consisting mainly of genes CD53, PTPRC, LAPTM5, DOCK2, EVI2B, CYBB and LCP2. This attractor is strongly associated with the expression of miR-142 as well as with particular hypermethylated and hypomethylated gene signatures. (Andreopoulos, B. & Anastassiou, D., Cancer Informatics 11, 61-75 (2012)). The latter include many of the overexpressed genes, suggesting that their expression is triggered by hypomethylation. Gene set enrichment analysis reveals that the attractor is found enriched in genes known to be preferentially expressed in lymphocyte differentiation and is also found occasionally upregulated in various cancers. (Lee et al., International Immunology 16, 1109-1124 (2004)). Table 4 provides a listing of the top 100 genes of the lymphocyte-specific attractor based on their average mutual information with their corresponding metagenes.

TABLE 4
Top 100 genes of the lymphocyte-specific attractor based on six datasets

| | Gene | |
|---|---|---|
| Rank | Symbol | Avg MI |
| 1 | PTPRC | 0.782 |
| 2 | CD53 | 0.768 |
| 3 | LCP2 | 0.739 |
| 4 | LAPTM5 | 0.708 |
| 5 | DOCK2 | 0.699 |
| 6 | IL10RA | 0.699 |
| 7 | CYBB | 0.698 |
| 8 | CD48 | 0.691 |
| 9 | ITGB2 | 0.679 |
| 10 | EVI2B | 0.675 |
| 11 | MS4A6A | 0.673 |
| 12 | TFEC | 0.659 |
| 13 | SLA | 0.657 |
| 14 | TNFSF13B | 0.657 |
| 15 | C1orf162 | 0.656 |
| 16 | SAMSN1 | 0.652 |
| 17 | PLEK | 0.649 |
| 18 | GMFG | 0.647 |
| 19 | GIMAP4 | 0.647 |
| 20 | SASH3 | 0.645 |
| 21 | EVI2A | 0.638 |
| 22 | SRGN | 0.638 |
| 23 | AIF1 | 0.636 |
| 24 | LAIR1 | 0.627 |
| 25 | FYB | 0.625 |
| 26 | FCER1G | 0.623 |
| 27 | MPEG1 | 0.621 |
| 28 | CD86 | 0.621 |
| 29 | C3AR1 | 0.611 |
| 30 | C1QB | 0.608 |
| 31 | CD2 | 0.606 |
| 32 | HCLS1 | 0.599 |
| 33 | HCK | 0.592 |
| 34 | MNDA | 0.587 |
| 35 | CD37 | 0.587 |
| 36 | LY96 | 0.585 |
| 37 | CCR5 | 0.585 |
| 38 | ARHGAP9 | 0.580 |
| 39 | CD52 | 0.580 |
| 40 | GPR65 | 0.580 |
| 41 | GIMAP6 | 0.578 |
| 42 | SLAMF8 | 0.577 |
| 43 | WIPF1 | 0.577 |
| 44 | MS4A4A | 0.574 |
| 45 | ARHGAP15 | 0.573 |
| 46 | HAVCR2 | 0.567 |
| 47 | ARHGAP30 | 0.566 |
| 48 | CLEC4A | 0.566 |
| 49 | TAGAP | 0.564 |
| 50 | CYTIP | 0.563 |
| 51 | NCF1 | 0.560 |
| 52 | CCL5 | 0.557 |
| 53 | LST1 | 0.557 |
| 54 | CD3D | 0.553 |
| 55 | RCSD1 | 0.548 |
| 56 | FGL2 | 0.538 |
| 57 | HCST | 0.538 |
| 58 | MARCH1 | 0.538 |
| 59 | FERMT3 | 0.536 |
| 60 | FCGR2B | 0.533 |
| 61 | GIMAP5 | 0.530 |
| 62 | MYOIF | 0.530 |
| 63 | KLHL6 | 0.530 |
| 64 | GIMAP1 | 0.527 |
| 65 | CD163 | 0.524 |
| 66 | CLEC7A | 0.522 |
| 67 | CCR1 | 0.518 |
| 68 | GBP5 | 0.517 |
| 69 | NCF2 | 0.516 |
| 70 | HLA- DPA1 | 0.516 |
| 71 | RNASE6 | 0.515 |
| 72 | CD14 | 0.515 |
| 73 | FAM26F | 0.511 |
| 74 | CD4 | 0.510 |
| 75 | FCGR1A | 0.506 |
| 76 | GZMA | 0.506 |
| 77 | GPR183 | 0.505 |
| 78 | CD84 | 0.505 |
| 79 | NKG7 | 0.504 |
| 80 | C1QA | 0.502 |
| 81 | CD300LF | 0.500 |
| 82 | FPR3 | 0.499 |
| 83 | PARVG | 0.496 |
| 84 | TRAF3IP3 | 0.494 |
| 85 | TYROBP | 0.492 |
| 86 | LPXN | 0.492 |
| 87 | GIMAP8 | 0.492 |
| 88 | MS4A7 | 0.490 |
| 89 | IL2RB | 0.489 |
| 90 | CD300A | 0.488 |
| 91 | IGSF6 | 0.488 |
| 92 | SELPLG | 0.488 |
| 93 | FCGR2A | 0.487 |
| 94 | NCKAP1L | 0.483 |
| 95 | DOK2 | 0.483 |
| 96 | CD247 | 0.481 |
| 97 | SELL | 0.480 |
| 98 | GZMK | 0.479 |
| 99 | CCR2 | 0.479 |
| 100 | LY86 | 0.479 |

4.5. Chr8q24.3 Amplicon Attractor Metagene

Amplification in chr8q24 is often associated with cancer because of the presence of the MYC (aka c-Myc) oncogene at location 8q24.21. Indeed, MYC is one of 157 genes in “amplicon 8q23-q24” previously identified in an extensive study of the breast cancer “amplicome” derived from 191 samples. (Nikolsky et al., Cancer Res 68, 9532-9540 (2008)).

It was found, however, that the core of the amplified genes occurs at location 8q24.3 and this is, in fact, the most prominent multi-cancer amplicon attractor. The main core gene of the attractor appears to be PUF60 (aka FIR). Other consistently present top genes are EXOSC4, CYC 1, SHARPIN, HSF1, GPR172A. It is known that PUF60 can repress c-Myc via its far upstream element (FUSE), although a particular isoform was found have the opposite effect. (Matsushita et al., Cancer Res 66, 1409-1417 (2006)). The other genes may also play important roles. For example, HSF1 (heat shock transcription factor 1) has been associated with cancer in various ways. (Dai et al., Cell 130, 1005-1018 (2007). It was found that HSF1 can induce genomic instability through direct interaction with CDC20, a key gene of the mitotic CIN attractor mentioned above (listed in Table 3). (Lee et al., Oncogene 27, 2999-3009 (2008)). Furthermore, HSF1 was found required for the cell transformation and tumorigenesis induced by the ERBB2 (aka HER2) oncogene (see subsequent discussion of HER2 amplicon) responsible for aggressive breast tumors. (Meng et al., Oncogene 29, 5204-5213 (2010)).

4.6. Chr17q12 HER2 Amplicon Attractor Metagene

This amplicon is prominent in breast cancer but was also found to be present in some samples of ovarian cancer, but not as much in colon cancer. (Theillet, Breast Cancer Res 12, 107 (2010)). Among the top four genes of the attractor are ERBB2 (aka HER2), GRB7 and STARD3, consistent with their known presence in the amplicon. However, MIEN1 (aka C17orf37) was also identified to have equal strength in the attractor as these three genes. This gene has also recently been identified as an important player within the 17q12 amplicon in various cancers including prostate cancer. (Dasgupta et al., Oncogene 28, 2860-2872 (2009)).

The HER2 amplicon is known to contain multiple focal amplifications of neighboring loci. For example, in addition to the narrow HER2 amplicons, sometimes a large amplicon extends to more than a million bases containing both HER2 as well as TOP2A (one of the genes of the mitotic chromosomal instability attractor) at 17q21. (Arriola, et al., Lab Invest 88, 491-503 (2008)). This is confirmed in the instant results from the existing, though weak, correlation of TOP2A with the HER2 amplicon. HER2/TOP2A co-amplification has been linked with better clinical response to therapy.

4.7. Diagnosis & Treatment Employing Attractor Metagenes

4.7.1. Methods of Diagnosis & Treatment Generally

Conventional gene expression analysis in connection with cancer diagnosis and treatment has resulted in several cancer types being further classified into subtypes labeled, e.g. as “mesenchymal” or “proliferative.” Such characterizations, however, may sometimes simply reflect the presence of the mesenchymal transition attractor or the mitotic chromosomal instability attractor, respectively, in some of the analyzed samples. Similar subtype characterizations across cancer types often share several common genes, but the consistency of these similarities has not been significantly high.

In contrast, by using an unconstrained algorithm independent of subtype classification or dimensionality reduction, as described herein, several attractors exhibiting remarkable consistency across many cancer types can be identified, indicating that each of them represents a precise biological phenomenon present in multiple cancers and therefore are of particular use in cancer diagnosis and treatment.

For example, the mesenchymal transition attractor described above is significantly present only in samples whose stage designation has exceeded a threshold, but not in all of such samples. Similarly, the mitotic chromosomal instability attractor described above is significantly present only in samples whose grade designation has exceeded a threshold, but not in all of them. On the other hand, the absence of the mesenchymal transition attractor in a profiled high-stage sample (or the absence of the mitotic chromosomal instability attractor in a profiled high-grade sample) does not necessarily mean that the attractor is not present in other locations of the same tumor. Indeed, it is increasingly appreciated that tumors are highly heterogeneous. (Gerlinger et al., The New England Journal of Medicine 366, 883-892 (2012)). Therefore it is possible for the same tumor to contain components, in which, e.g., some are migratory having undergone mesenchymal transition, some other ones are highly proliferative, etc. If so, attempts for subtype classification based on one particular site in a sample may be confusing.

Similarly, existing molecular marker products make use of multigene assays that have been derived from phenotypic associations in particular cancer types. For breast cancer, biomarkers such as Oncotype DX (Paik et al., The New England Journal of Medicine 351, 2817-2826 (2004)) and Mammaprint (van't Veer et al., Nature 415, 530-536 (2002)) contain several genes highly ranked in the attractors. For example, many among the genes used for the Oncotype DX breast cancer recurrence score directly converge to one of the identified attractors: MMP11 to the mesenchymal transition attractor; MKI67 (aka Ki-67), AURKA (aka STK15), BIRC5 (aka Survivin), CCNB1, and MYBL2 to the mitotic chromosomal instability attractor; CD68 to the lymphocyte-specific attractor; ERBB2 and GRB7 to the HER2 amplicon attractor; and ESR1, SCUBE2, PGR to the ESR1 attractor.

In contrast, the present invention relates, in certain embodiments, to a “multidimensional” biomarker product that will be applicable to multiple cancer types. Each of the dimensions of such embodiments will correspond to a specific attractor detected from a sharp choice of the gene at its core, reflecting a precise biological attribute of cancer. For example, each relevant amplicon can be identified by the coordinate co-expression of the top few genes of the attractor without any need for sequencing, and each will correspond to another dimension. The collection of the independent results in many dimensions will provide a clearer diagnostic and prognostic image after cleanly distinguishing the contributions of each component, whether the embodiment is directed to cancer or any other indication. Thus, even though molecular marker genes in existing products are often separated into groups that are related to the attractor designation, the improvement in diagnostic, prognostic, or predictive accuracy resulting from better such group designation and better choice of genes in each group that is achieved using the methods and compositions described herein is highly desirable.

4.7.2. Methods of Using Attractor Metagenes for Diagnosis and/or Treatment

In certain embodiments, the present invention provides for methods of treating a subject, such as, but not limited to, methods comprising performing a diagnostic method as set forth herein and then, if an attractor metagene is detected in a sample of the subject, administering therapy consistent with the presence or absence of the attractor metagene.

In certain non-limiting embodiments of the present invention, a diagnostic method as set forth above is performed and a therapeutic decision is made in light of the results of that diagnostic method. For example, but not by way of limitation, a therapeutic decision, such as whether to prescribe a particular therapeutic or class of therapeutic can be made in light of the results of a diagnostic method as set forth below. The results of the diagnostic methods described herein are relevant to the therapeutic decision as the presence of the attractor metagene or a subset of markers associated with it, in a sample from a subject can, in certain embodiments, indicate a decrease in the relative benefit conferred by a particular therapeutic intervention.

In certain embodiments, a diagnostic method as set forth below is performed and a decision regarding whether to continue a particular therapeutic regimen is made in light of the results of that diagnostic method. For example, but not by way of limitation, a decision whether to continue a particular therapeutic regimen, such as whether to continue with one or more of the therapeutics described herein can be made in light of the results of a diagnostic method as set forth below. The results of the diagnostic method are relevant to the decision whether to continue a particular therapeutic regimen as the presence of the attractor metagene or a subset of markers associated with it, in a sample from a subject can be indicative of the subject's responsiveness to the particular therapeutic.

In certain embodiments, the present invention provides for methods of performing a prognosis of a subject identified as having cancer, such as, but not limited to, methods comprising performance of a diagnostic method as set forth herein (e.g., obtaining a sample from the subject and determining whether an attractor metagene can be detected in the sample) and then, if an attractor metagene is detected in a sample of the subject, predicting the likely outcome (i.e., performing a prognosis) of the cancer, e.g., the likely survival duration. In certain embodiments, the prognosis will be based on the presence of one or more attractor metagenes. In certain embodiments, the prognosis will be based on the presence of one or more attractor metagenes and one or more additional factors, such as clinical and molecular features (e.g., the number of cancer-positive lymph nodes, age at diagnosis, and expression levels of particular genes exhibiting protective activity).

In certain embodiments, biomarker assays capable of identifying an attractor metagenes in patient samples for use in connection with the therapeutic interventions discussed herein can include, but are not limited to, nucleic acid amplification assays; nucleic acid hybridization assays; as well as protein detection assays that are specific for the attractor metagene biomarkers discussed herein. In certain embodiments, the assays of the present invention involve combinations of such detection techniques, e.g., but not limited to: assays that employ both amplification and hybridization to detect a change in the expression, such as overexpression or decreased expression, of a gene at the nucleic acid level; immunoassays that detect a change in the expression of a gene at the protein level; as well as combination assays comprising a nucleic acid-based detection step and a protein-based detection step.

A “sample” from a subject to be tested according to one of the assay methods described herein can be at least a portion of a tissue, at least a portion of a tumor, a cell, a collection of cells, or a fluid (e.g., blood, cerebrospinal fluid, urine, expressed prostatic fluid, peritoneal fluid, a pleural effusion, peritoneal fluid, etc.). In certain embodiments the sample used in connection with the assays of the instant invention will be obtained via a biopsy. Biopsy can be done by an open or percutaneous technique. Open biopsy is conventionally performed with a scalpel and can involve removal of the entire tumor mass (excisional biopsy) or a part of the tumor mass (incisional biopsy). Percutaneous biopsy, in contrast, is commonly performed with a needle-like instrument either blindly or with the aid of an imaging device, and can be either a fine needle aspiration (FNA) or a core biopsy. In FNA biopsy, individual cells or clusters of cells are obtained for cytologic examination. In core biopsy, a core or fragment of tissue is obtained for histologic examination which can be done via a frozen section or paraffin section.

“Overexpression” and “increased activity”, as used herein, refers to an increase in expression or activity, respectively, of a gene product relative to a normal or control value, which, in non-limiting embodiments, is an increase of at least about 30% or at least about 40% or at least about 50%, or at least about 100%, or at least about 200%, or at least about 300%, or at least about 400%, or at least about 500%, or at least 1000%.

“Decreased expression” and “decreased activity”, as used herein, refers to an decrease in expression or activity, respectively, of a gene product relative to a normal or control value, which, in non-limiting embodiments, is an decrease of at least about 30% or at least about 40% or at least about 50%, at least about 90%, or a decrease to a level where the expression or activity is essentially undetectable using conventional methods.

As used herein, a “gene product” refers to any product of transcription and/or translation of a gene. Accordingly, gene products include, but are not limited to, microRNA, pre-mRNA, mRNA, and proteins.

In certain embodiments, the present invention provides compositions and methods for the detection of gene expression indicative of all or part of the attractor metagene in a sample using nucleic acid hybridization and/or amplification-based assays.

In non-limiting embodiments, the genes/proteins within the attractor metagene set forth above constitute at least 10 percent, or at least 20 percent, or at least 30 percent, or at least 40 percent, or at least 50 percent, or at least 60 percent, or at least 70 percent, or at least 80 percent, or at least 90 percent, of the genes/proteins being evaluated in a given assay.

In certain embodiments, the present invention provides compositions and methods for the detection of gene expression indicative of all or part of the attractor metagene in a sample using a nucleic acid hybridization assay, wherein nucleic acid from said sample, or amplification products thereof, are hybridized to an array of one or more nucleic acid probe sequences. In certain embodiments, an “array” comprises a support, preferably solid, with one or more nucleic acid probes attached to the support. Preferred arrays typically comprise a plurality of different nucleic acid probes that are coupled to a surface of a substrate in different, known locations. These arrays, also described as “microarrays” or “chips” have been generally described in the art, for example, U.S. Pat. Nos. 5,143,854, 5,445,934, 5,744,305, 5,677,195, 5,800,992, 6,040,193, 5,424,186 and Fodor et al., Science, 251:767-777 (1991).

Arrays can generally be produced using a variety of techniques, such as mechanical synthesis methods or light directed synthesis methods that incorporate a combination of photolithographic methods and solid phase synthesis methods. Techniques for the synthesis of these arrays using mechanical synthesis methods are described in, e.g., U.S. Pat. Nos. 5,384,261, and 6,040,193, which are incorporated herein by reference in their entirety for all purposes. Although a planar array surface is preferred, the array can be fabricated on a surface of virtually any shape or even a multiplicity of surfaces. Arrays can be nucleic acids on beads, gels, polymeric surfaces, fibers such as fiber optics, glass or any other appropriate substrate. See U.S. Pat. Nos. 5,770,358, 5,789,162, 5,708,153, 6,040,193 and 5,800,992.

In certain embodiments, the arrays of the present invention can be packaged in such a manner as to allow for diagnostic, prognostic, and/or predictive use or can be an all-inclusive device; e.g., U.S. Pat. Nos. 5,856,174 and 5,922,591.

In certain embodiments, the hybridization assays of the present invention comprise a primer extension step. Methods for extension of primers from solid supports have been disclosed, for example, in U.S. Pat. Nos. 5,547,839 and 6,770,751. In addition, methods for genotyping a sample using primer extension have been disclosed, for example, in U.S. Pat. Nos. 5,888,819 and 5,981,176.

In certain embodiments, the methods for detection of all or a part of the attractor metagene in a sample involves a nucleic acid amplification-based assay. In certain embodiments, such assays include, but are not limited to: real-time PCR (for example see Mackay, Clin. Microbiol. Infect. 10(3):190-212, 2004), Strand Displacement Amplification (SDA) (for example see Jolley and Nasir, Comb. Chem. High Throughput Screen. 6(3):235-44, 2003), self-sustained sequence replication reaction (3SR) (for example see Mueller et al., Histochem. Cell. Biol. 108(4-5):431-7, 1997), ligase chain reaction (LCR) (for example see Laffler et al., Aim. Biol. Clin. (Paris).51(9):821-6, 1993), transcription mediated amplification (TMA) (for example see Prince et al., J. Viral Hepat. 11(3):236-42, 2004), or nucleic acid sequence based amplification (NASBA) (for example see Romano et al., Clin. Lab. Med. 16(1):89-103, 1996).

In certain embodiments of the present invention, a PCR-based assay, such as, but not limited to, real time PCR is used to detect the presence of an attractor metagene in a test sample. In certain embodiments, attractor metagene-specific PCR primer sets are used to amplify attractor metagene-associated RNA and/or DNA targets. Signal for such targets can be generated, for example, with fluorescence-labeled probes. In the absence of such target sequences, the fluorescence emission of the fluorophore can be, in certain embodiments, eliminated by a quenching molecule also operably linked to the probe nucleic acid. However, in the presence of the target sequences, probe binds to template strand during primer extension step and the nuclease activity of the polymerase catalyzing the primer extension step results in the release of the fluorophore and production of a detectable signal as the fluorophore is no longer linked to the quenching molecule. (Reviewed in Bustin, J. Mol. Endocrinol 25, 169-193 (2000)). The choice of fluorophore (e.g., FAM, TET, or Cy5) and corresponding quenching molecule (e.g. BHQ1 or BHQ2) is well within the skill of one in the art and specific labeling kits are commercially available.

In certain embodiments, the present invention provides compositions and methods for the detection of gene expression indicative of all or part of the attractor metagene in a sample by employing high throughput sequencing techniques, such as RNA-seq. (See, e.g., Wang et al., RNA-Seq: a revolutionary tool for transcriptomics, Nat Rev Genet. 2009 January; 10(1): 57-63). In general, such techniques involve obtaining a sample population of RNA (total or fractionated, such as poly(A)+) which is then converted to a library of cDNA fragments, typically of 30-400 bp in length. These cDNA fragments will be generated to include adaptors attached to one or both ends, depending on whether the subsequent sequencing step proceeds from one or both ends. Each of the adaptor-tagged molecules, with or without amplification, can then be sequenced in a high-throughput manner to obtain short sequences. Virtually any high-throughput sequencing technology can be used for the sequencing step, including, but not limited to the Illumina IG®, Applied Biosystems SOLiD®, Roche 454 Life Science®, and Helicos Biosciences tSMS® systems. Following sequencing, bioinformatics techniques can be used to either align there results against a reference genome or to assemble the results de novo. Such analysis is capable of identifying both the level of expression for each gene as well as the sequence of particular expressed genes.

In certain embodiments, the present invention provides compositions and methods for the detection of gene expression indicative of all or part of the attractor metagene in a sample by detecting changes in concentration of the protein, or proteins, encoded by the genes of interest.

In certain embodiments, the present invention relates to the use of immunoassays to detect modulation of gene expression by detecting changes in the concentration of proteins expressed by a gene of interest. Numerous techniques are known in the art for detecting changes in protein expression via immunoassays. (See The Immunoassay Handbook, 2nd Edition, edited by David Wild, Nature Publishing Group, London 2001.) In certain of such immunoassays, antibody reagents capable of specifically interacting with a protein of interest, e.g., an individual member of the attractor metagene, are covalently or non-covalently attached to a solid phase. Linking agents for covalent attachment are known and can be part of the solid phase or derivatized to it prior to coating. Examples of solid phases used in immunoassays are porous and non-porous materials, latex particles, magnetic particles, microparticles, strips, beads, membranes, microtiter wells and plastic tubes. The choice of solid phase material and method of labeling the antibody reagent are determined based upon desired assay format performance characteristics. For some immunoassays, no label is required, however in certain embodiments, the antibody reagent used in an immunoassay is attached to a signal-generating compound or “label”. This signal-generating compound or “label” is in itself detectable or can be reacted with one or more additional compounds to generate a detectable product (see also U.S. Pat. No. 6,395,472 B1). Examples of such signal generating compounds include chromogens, radioisotopes (e.g., 125I, 131I, 32P, 3H, 35S, and 14C), fluorescent compounds (e.g., fluorescein and rhodamine), chemiluminescent compounds, particles (visible or fluorescent), nucleic acids, complexing agents, or catalysts such as enzymes (e.g., alkaline phosphatase, acid phosphatase, horseradish peroxidase, beta-galactosidase, and ribonuclease). In the case of enzyme use, addition of chromo-, fluoro-, or lumo-genic substrate results in generation of a detectable signal. Other detection systems such as time-resolved fluorescence, internal-reflection fluorescence, amplification (e.g., polymerase chain reaction) and Raman spectroscopy are also useful in the context of the methods of the present invention.

In certain embodiments, the assays of the present invention are capable of detecting coordinated modulation of expression, for example, but not limited to, overexpression, of the genes associated with the attractor metagene. In certain embodiments, such detection involves, but is not limited to, detection of the expression of one or more of the attractor metagenes identified in FIGS. 1A-1B.

Any of the exemplary assay formats described herein can be adapted or optimized for use in automated and semi-automated systems (including those in which there is a solid phase comprising a microparticle), for example as described, e.g., in U.S. Pat. Nos. 5,089,424 and 5,006,309, and in connection with any of the commercially available detection platforms known in the art.

In certain embodiments, the methods and/or assays of the present invention are directed to the detection of all or a part of the attractor metagene wherein such detection can take the form of either a binary, detected/not-detected, result. In certain embodiments, the methods, assays, and/or kits of the present invention are directed to the detection of all or a part of the attractor metagene wherein such detection can take the form of a multi-factorial result. For example, but not by way of limitation, such multi-factorial results can take the form of a score based on one, two, three, or more factors. Such factors can include, but are not limited to: (1) detection of a change in expression of an attractor metagene gene product, state of methylation, and/or presence of microRNA; (2) the number of attractor metagene gene products, states of methylation, and/or presence of microRNAs in a sample exhibiting an altered level; and (3) the extent of such change in attractor metagene gene products, states of methylation, and/or presence of microRNAs.

4.7.3. Kits Comprising Attractor Metagenes for Diagnosis and/or Treatment

In certain embodiments, compositions useful in the detection and/or assaying of one or more attractor metagene of the present invention can be packaged into kits.

In certain embodiments, a kit may comprise a pair of oligonucleotide primers, suitable for polymerase chain reaction, for each gene and/or gene product to be measured. Such primers may be designed based on the sequences for the genes associated with said attractor metagene(s).

In certain embodiments the kit will include a measurement means, such as, but not limited to a microarray. In certain non-limiting embodiments, where the measurement means in the kit employs a microarray, the set of markers associated with the attractor metagene may constitute at least 10 percent or at least 20 percent or at least 30 percent or at least 40 percent or at least 50 percent or at least 60 percent or at least 70 percent or at least 80 percent of the species of markers represented on the chip.

Any of the foregoing kits, in this or the preceding sections, may further optionally comprise one or more controls such as a healthy control, or any other appropriate control to allow for diagnosis. In non-limiting examples, such controls may be plasma samples or may be combinations of genes and/or gene products prepared to resemble such natural plasma samples.

### 5. EXAMPLES

### 5.1 Example 1

### 5.1.1. Materials & Methods

5.1.1.1. General attractor finding algorithm

The association measure J(Gᵢ, Gⱼ) between genes was chosen to be a power function with exponent a of a normalized estimated information theoretic measure of the mutual information I(Gᵢ, Gⱼ) with minimum value 0 and maximum value 1, as a proper compromise between performance and complexity (more sophisticated related association measures can also be used). In other words, J(Gᵢ, Gⱼ)=I^{a}(Gᵢ, Gⱼ), in which the exponent a can be any nonnegative number. As described in Results section, each iteration of the algorithm will define a new metagene in which the weight wᵢ for gene Gᵢ will be equal to wᵢ=J(Gᵢ, M), where M is the immediately preceding metagene. The process is repeated until the magnitude of the difference between two consecutive weight vectors is less than a threshold, which was chosen in this instance to be equal to 10⁻⁷.

At one extreme, if a is very large then each of the seeds will create its own single-gene attractor because all other genes will always have near-zero weights. In that case, the total number of attractors will be equal to the number of genes. At the other extreme, if a is zero then all weights will remain equal to each other, thus representing the average of all genes, so there will only be one attractor. The higher the value of a, the “sharper” (more focused on its top gene) each attractor will be and the higher the overall number of attractors will be. As the value of a is gradually decreased, the attractor from a particular seed will transform itself, occasionally in a discontinuous manner, thus providing insight into potential related biological mechanisms.

An appropriate choice of a was empirically found (in the sense of revealing single biomolecular events of co-expressed genes) for general attractors is around 5, in which case there will typically be approximately 50 to 150 resulting attractors, each resulting from numerous attractee genes, depending on the number of genes and the cancer type. (An alternative to the power function can be a sigmoid function with varying steepness, but the consistency of the resulting attractors was found to be slightly worse in that case).

An attractor metagene can also be interpreted as a set of co-expressed genes containing a number among the top genes of the attractor. In that case, one can define the size of such set so that the set contains only the genes that are significantly associated with the attractor. One empirical such criterion would be to include the genes whose z-score of their mutual information with the attractor exceeds a large threshold, such as 20.

Identified attractors can be ranked in various ways. The “strength of an attractor” can be defined as the mutual information between the n^{th} top gene of the attractor and the attractor metagene itself. Indeed, if this measure is high, this implies that at least the top n genes of the attractor are strongly co-expressed. For example, n=50 can be a reasonable choice, not too large, but sufficiently so to represent a real complex biological phenomenon of co-expression of at least 50 genes. For amplicons, n=5 is sufficient to ensure that the oncogenes are included in the co-expression). These choices are employed when referring to the strength of an attractor.

The top genes of many among the found attractors are in identical chromosomal locations. In that case the biomolecular event that they represent is the presence of a particular copy number variation. In the cancer datasets that were analyzed, this phenomenon almost always corresponds to a local amplification event known as an amplicon. A related amplicon-finding algorithm, custom-designed to identify localized amplicon-representing attractor metagenes, was also devised as described below.

5.1.1.2. Amplicon Finding Algorithm

To identify amplicons the same algorithm is employed, but for each seed gene the set of candidate attractor genes is restricted to only include those in the local genomic neighbourhood of the gene, and the exponent is selected a so that the strength of the attractor is maximized. Specifically, the genes in each chromosome are sorted in terms of their genomic location and only the genes within a window of size 51, i.e., with 25 genes on each side of the seed gene, are considered. The choice of the exponent a for each seed is also selected, by allowing a to range from 1.0 to 6.0 with step size of 0.5 and identifying the attractor with the highest strength.

Because the set of allowed genes is different for each seed, the attractors will be different from each other, but “neighbouring” attractors will usually be very similar to each other. Therefore, following exhaustive attractor finding by considering each seed gene in a chromosome, a filtering algorithm is applied to only select the highest-strength attractor in each local genomic region, as follows: For each attractor, all the genes are ranked in terms of their mutual information with the corresponding attractor metagene and the range of the attractor to be the chromosomal range of its top 15 genes is determined. If there is any other attractor with overlapping range and higher strength, then the former attractor will be filtered out. This filtering is done in parallel so elimination of attractors occurs simultaneously. The remaining “winning” attractors are assumed to correspond to real amplicons. Of course, the co-expression of the genes in such attractors will still occasionally be due to other co-regulation biological mechanisms, as in the local region of a major histocompatibility complex. They may also be due to copy number deletions, rather than amplifications. In all cases, however, the resulting locally focused attractors will still be interesting.

5.1.1.3. Mutual Information Estimation

Assuming that the continuous expression levels of two genes G₁ and G₂ are governed by a joint probability density p₁₂ with corresponding marginals p₁ and p₂ and using simplified notation, the mutual information [symbol] I(G [symbol] ₁, G₂) is defined as the expected value of log(p₁₂/p₁p₂). It is a non-negative quantity representing the information that each one of the variables provides about the other. The pairwise mutual information has successfully been used as a general measure of the correlation between two random variables. Mutual information is computed with a spline-based estimator using six bins in each dimension. This method divides the observation space into equally spaced bins and blurs the boundaries between the bins with spline basis functions using third-order B-splines. Normalization of the estimated mutual information is accomplished by dividing by the maximum of the estimated [symbol] I(G [symbol] ₁, G₂) and [symbol] I(G [symbol] ₁, G₂), so the maximum possible value of [symbol] I(G [symbol] ₁, G₂) is 1.

5.1.1.4. Pre-Processing Gene Expression Datasets

Among the list of datasets in Table 1, Level 3 data was used when directly available, and imputed missing values using a k-nearest-neighbour algorithm with k=10, as implemented in Troyanskaya et al., Bioinformatics 17, 520-525 (2001). The other datasets on the Affymetrix platform were normalized using the RMA algorithm as implemented in the Affymetrix package in Bioconductor.

To avoid biasing attractor convergence with multiple correlated probe sets of the same gene, the probe set-level expression values were summarized into the gene-level expression values by taking the mean of the expression values of probe sets for the same genes. The annotations for the probe sets are given in the jetset package. (Li et al., BMC Bioinformatics 12, 474 (2011)).

To investigate the associations between the attractor metagene expression and the tumor stage and grade, the following annotated gene expression datasets were used. For stage association: Breast (GSE3893), TCGA Ovarian, Colon (GSE14333). For grade association: Breast (GSE3494), TCGA Ovarian, Bladder (GSE13507). For Breast GSE3494 only the samples profiled by U133A arrays were used. For Breast GSE3893 two platforms were combined by taking the intersections of the probes in the U133A and the U133Plus 2.0 arrays. For datasets profiled by Affymetrix platforms all the datasets were normalized using the RMA algorithm. For Bladder GSE13507 normalization was provided in the dataset.

5.1.1.5. Clustering Attractors in Multiple Datasets

After applying the attractor finding algorithms in the six datasets of Table 1, any attractors that resulted from less than three attractee (seed) genes were filtered out. To identify common attractors in different datasets, the genes were first ranked in each attractor according to their mutual information with the attractor metagene, selecting the top 50 genes as its representative “attractor gene set.” Hierarchical clustering on the attractor gene sets was then performed. The clustering algorithm iteratively defines “attractor clusters,” each of which only contains attractors from distinct datasets (i.e. its maximum size is six). The “similarity score” between two attractor clusters is defined to be the number of overlapping genes among all possible pairs of attractor gene sets between two attractor clusters. If two attractor clusters both contain gene sets from the same datasets, then they are not clustered together. Starting from the two attractor gene sets with highest similarity score, the process proceeded until there was no attractor cluster pair that could be further clustered together.

5.1.1.6. Clustering Amplicon Attractors in Multiple Datasets

All amplicon attractors were ranked in each dataset according to their strength and perform the same clustering algorithm as described above, except that attractor gene sets have size 15 and the similarity score is set to 1 if two attractors are overlapping and 0 if their ranges are exclusive.

### 5.1.2. Results & Discussion

5.1.2.1. Mesenchymal Transition Attractor Metagene

This attractor contains mostly epithelial-mesenchymal transition (EMT)-associated genes. Table 2, presented above, provides a listing of top 100 genes based on their average mutual information with their corresponding attractor metagenes.

This is a stage-associated attractor, in which the signature is significantly present only when a particular level of invasive stage, specific to each cancer type, has been reached. This phenomenon is observed, in three cancer datasets from different types (breast, ovarian and colon) that were annotated with clinical staging information, by providing a listing of differentially expressed genes, ranked by fold change, when ductal carcinoma in situ (DCIS) progresses to invasive ductal carcinoma; colon cancer progresses to stage II; and ovarian cancer progresses to stage III. In all three cases, the attractor is highly enriched among the top genes. Specifically, among the top 100 differentially expressed genes, the number of attractor genes included Table 2 were 55 in breast cancer, 45 in ovarian cancer and 31 in colon cancer. The corresponding Fisher's exact test P values are 3×10⁻¹⁰⁹, 9×10⁻⁸³ and 5×10⁻⁶², respectively.

This attractor has been previously identified with remarkable accuracy as representing a particular kind of mesenchymal transition of cancer cells present in all types of solid cancers tested leading to a published list of top 64 genes. Indeed 56 of these top 64 genes appear in Table 2 (P<10⁻¹²⁷), and all top 24 genes of Table 2 are among the 64. Most of the genes of the signature were found to be expressed by the cancer cells themselves, and not by the surrounding stroma, at least in a neuroblastoma xenograft model. The signature is found to be associated with prolonged time to recurrence in glioblastoma. Related versions of the same signature were previously found to be associated with resistance to neoadjuvant therapy in breast cancer. These results are consistent with the finding that EMT induces cancer cells to acquire stem cell properties. It has been hypothesized that EMT is a key mechanism for cancer cell invasiveness and motility. The attractor, however, appears to represent a more general phenomenon of transdifferentiation present even in nonepithelial cancers such as neuroblastoma, glioblastoma and Ewing's sarcoma.

Although similar signatures are often labeled as “stromal,” because they contain many stromal markers such as α-SMA and fibroblast activation protein, the fact that most of the genes of the signature were expressed by xenografted cancer cells, and not by mouse stromal cells, suggests that this particular attractor of coordinately expressed genes represents cancer cells having undergone a mesenchymal transition. The signature may indicate a non-fibroblastic transition, as occurs in glioblastoma, in which case collagen COL11A1 is not co-expressed with the other genes of the attractor. It is believed that a full fibroblastic transition of the cancer cells occurs when cancer cells encounter adipocytes, in which case they may well assume the duties of cancer associated fibroblasts (CAFs) in some tumors. In that case, the best proxy of the signature is COL11A1 and the strongly co-expressed genes THBS2 and INHBA. Indeed, the 64 genes of the previously identified signature were found from multi-cancer analysis as the genes whose expression is consistently most associated with that of COL11A1.

The only EMT-inducing transcription factor found upregulated in the xenograft model is SNAI2 (Slug), and it is also the one most associated with the signature in publicly available datasets. The microRNAs found to be most highly associated with this attractor are miR 214, miR 199a, and miR-199b. Interestingly, miR-214 and miR-199a were found to be jointly regulated by another EMT-inducing transcription factor, TWIST1.

5.1.2.2. Mitotic CIN Attractor Metagene

This attractor contains mostly kinetochore-associated genes. Table 3, presented above, provides a listing of top 100 genes based on their average mutual information with their corresponding attractor metagenes.

Contrary to the stage associated mesenchymal transition attractor, this is a grade associated attractor, in which the signature is significantly present only when an intermediate level of tumor grade is reached. This phenomenon can be observed, in three cancer datasets from different types (breast, ovarian and bladder) that were annotated with tumor grade information, by providing a listing of differentially expressed genes, ranked by fold change, when grade G2 is reached. In all three cases, the attractor is highly enriched among the top genes. Specifically, among the top 100 differentially expressed genes, the number of attractor genes included Table 3 were 41 in breast cancer, 36 in ovarian cancer and 26 in colon cancer. The corresponding Fisher's exact test P values are 7×10⁻⁷³, 4×10⁻⁶¹ and 5×10⁻⁴⁷, respectively. Consistently, a similar “gene expression grade index” signature was previously found differentially expressed between histologic grade 3 and histologic grade 1 breast cancer samples. Furthermore, that same signature was found capable of reclassifying patients with histologic grade 2 tumors into two groups with high versus low risks of recurrence.

This attractor is associated with chromosomal instability (CIN), as evidenced from the fact that another similar gene set comprising a “signature of chromosomal instability” was previously derived from multiple cancer datasets purely by identifying the genes that are most correlated with a measure of aneuploidy in tumor samples. This led to a 70-gene signature referred to as “CIN70.” Indeed 34 of these 70 genes appear in Table 3 (P<10⁻⁶¹). However, several top genes of the attractor, such as CENPA, KIF2C, BUB1 and CCNA2 are not present in the CIN70 list. Mitotic CIN is increasingly recognized as a widespread multi-cancer phenomenon.

The attractor is characterized by overexpression of kinetochore-associated genes, which is known to induce chromosomal instability (CIN) for reasons that are not clear. Overexpression of several of the genes of the attractor, such as the top gene CENPA, as well as MAD2L1 and TPX2, has also been independently previously found associated with CIN. Included in the mitotic CIN attractor are key components of mitotic checkpoint signaling, such as BUB1B, MAD2L1 (aka MAD2), CDC20, and TTK (MSP1). It was recently found that the CIN70 signature is most strongly associated with poor outcome at intermediate, rather than extreme levels. This is consistent with the concept that, while cancer cells are intolerant of extreme instability, moderate mitotic chromosomal instability may provide a proliferative advantage.

Among transcription factors, MYBL2 (aka B-Myb) and FOXM1 were found to be strongly associated with the attractor. They are already known to be sequentially recruited to promote late cell cycle gene expression to prepare for mitosis.

Inactivation of the retinoblastoma (RB) tumor suppressor promotes CIN28 and the expression of the attractor signature. Indeed, a similar expression of a “proliferation gene cluster” was found strongly associated with the human papillomavirus E7 oncogene, which abrogates RB protein function and activates E2F-regulated genes. Consistently, many among the genes of the attractor correspond to E2F pathway genes controlling cell division or proliferation. Among the E2F transcription factors, E2F8 and E2F7 were found to be most strongly associated with the attractor.

5.1.2.4. A Lymphocyte-Specific Attractor Metagene

A strong lymphocyte-specific attractor was identified as consisting mainly of genes CD53, PTPRC, LAPTM5, DOCK2, EVI2B, CYBB and LCP2. This attractor is strongly associated with the expression of miR-142 as well as with particular hypermethylated and hypomethylated gene signatures. The latter include many of the overexpressed genes, suggesting that their expression is triggered by hypomethylation. Gene set enrichment analysis reveals that the attractor is found enriched in genes known to be preferentially expressed in lymphocyte differentiation and is also found occasionally upregulated in various cancers.

5.1.2.5. Chr8q24.3 Amplicon Attractor Metagene

Amplification in chr8q24 is often associated with cancer because of the presence of the MYC (aka c-Myc) oncogene at location 8q24.21. Indeed, MYC is one of 157 genes in “amplicon 8q23-q24” previously identified in an extensive study of the breast cancer “amplicome” derived from 191 samples. (Nikolsky et al., Cancer Res 68, 9532-9540 (2008)).

It was found, however, that the core of the amplified genes occurs at location 8q24.3 and this is, in fact, the most prominent multi-cancer amplicon attractor. The main core gene of the attractor appears to be PUF60 (aka FIR). Other consistently present top genes are EXOSC4, CYC1, SHARPIN, HSF1, GPR172A. It is known that PUF60 can repress c-Myc via its far upstream element (FUSE), although a particular isoform was found have the opposite effect. The other genes may also play important roles. For example, HSF1 (heat shock transcription factor 1) has been associated with cancer in various ways. It was found that HSF1 can induce genomic instability through direct interaction with CDC20, a key gene of the mitotic CIN attractor mentioned above (listed in Table 3). Furthermore, HSF1 was found required for the cell transformation and tumorigenesis induced by the ERBB2 (aka HER2) oncogene (see subsequent discussion of HER2 amplicon) responsible for aggressive breast tumors.

5.1.2.6. Chr17q12 HER2 Amplicon Attractor Metagene

This amplicon is prominent in breast cancer but was also found to be present in some samples of ovarian cancer, but not as much in colon cancer. Among the top four genes of the attractor are ERBB2 (aka HER2), GRB7 and STARD3, consistent with their known presence in the amplicon. However, MIEN1 (aka C17orf37) was also identified to have equal strength in the attractor as these three genes. This gene has also recently been identified as an important player within the 17q12 amplicon in various cancers including prostate cancer.

The HER2 amplicon is known to contain multiple focal amplifications of neighboring loci. For example, in addition to the narrow HER2 amplicons, sometimes a large amplicon extends to more than a million bases containing both HER2 as well as TOP2A (one of the genes of the mitotic chromosomal instability attractor) at 17q21. This is confirmed in the instant results from the existing, though weak, correlation of TOP2A with the HER2 amplicon. HER2/TOP2A co-amplification has been linked with better clinical response to therapy.

### 5.2. Example 2

### 5.2.1. Introduction

Medical tests that incorporate molecular profiling of tumors for clinical decision-making (predictive tests) or prognosis (prognostic tests) are typically based on models that combine values associated with particular molecular features, such as the expression levels of specific genes. These genes are selected after analyzing rich gene expression data sets (acquired from testing patient tumors) annotated with clinical phenotypes such as drug responses or survival times. The data sets used to define a model are referred to as “training data sets.” A computational technique is typically used to identify a number of genes that, when properly combined, are associated with a phenotype of interest in a statistically significant manner. The predictive power of the resulting model is later confirmed in independent “validation data sets.”

There are, however, vast numbers—tens or hundreds of thousands—of potentially relevant molecular features to choose from when developing a model, making it difficult to precisely identify those at the core of the biological mechanisms responsible for the phenotype of interest. Spurious or suboptimal predictions may occur, and the end result may be a model that only partly reflects physiological reality. Such a model may still be clinically useful, but there is room for improvement.

One way to address this problem is by using molecular features preselected on the basis of previous knowledge. In such an approach, a training data set is used mainly for pinpointing the combination of preselected features that is most associated with the phenotype of interest. The instant example describes the use of such an approach during in connection with the Sage Bionetworks—DREAM Breast Cancer Prognosis Challenge, an open challenge to build computational models that accurately predict breast cancer survival (hereinafter referred to as the Challenge). (Margolin et al., Sci. Transl. Med. 5, 181re1 (2013)). Specifically, selected gene coexpression signatures present in multiple cancer types, identified as attractor metagenes herein, were employed in the prediction of survival in breast cancer.

As outlined in the instant Example, certain attractor metagenes of the instant invention were strong prognostic features for breast cancer survival. This phenotypic association was present despite the fact that these attractor metagenes (i) were discovered by a purely unsupervised method (that is, without reference to any phenotypic association) and (ii) were determined without using the Challenge training data set. In addition, the instant Example outlines how such attractor metagenes can be combined with additional clinical and molecular features to predict patient ranking in terms of their survival.

### 5.2.2. Materials & Methods

5.2.2.1. A General Overview of Building A Prognostic Model

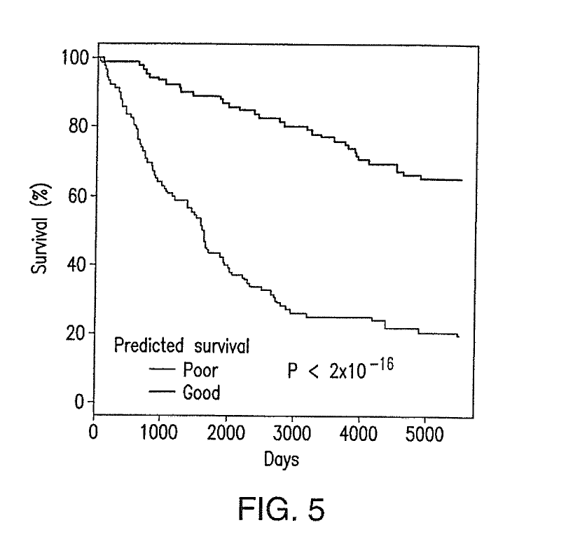

Building the prognostic model involved derivation and selection of relevant features, training the submodels using the derived features based on survival information, and combining predictions from the submodels illustrated in FIG. 5 to produce a robust ensemble prediction. In particular, FIG. 5 shows block diagrams describing an exemplary model and each subhead in the Figure corresponds to the section with the same subhead that follows.

5.2.2.2. Derivation of Features

The number of potential molecular features were reduced by preselecting the following 12 features due to their prognostic capability: (i) the CIN attractor metagene consisting of genes CENPA, DLGAP5, MELK, BUB1, KIF2C, KIF20A, KIF4A, CCNA2, CCNB2, and NCAPG; (ii) the MES attractor metagenes consisting of genes COL5A2, VCAN, SPARC, THBS2, FBN1, COL1A2, COL5A1, FAP, AEBP1, and CTSK; (iii) the LYM attractor metagenes consisting of genes PTPRC, CD53, LCP2, LAPTM5, DOCK2, IL10RA, CYBB, CD48, ITGB2, and EVI2B; (iv) the FGD3-SUSD3 metagene consisting of genes FGD3 and SUSD3 described in the Results section, below; (v) the chr8q24.3 amplicon attractor metagene consisting of genes EXOSC4, PUF60, BOP1, SLC52A2, SHARPIN, HSF1, FBXL6, CYC1, SCRIB, and GAPP1 (because it was found to be the most prominent amplicon in all cancer types previously analyzed and in the METABRIC training data set); (vi) the chr15q26.1 amplicon attractor metagene consisting of gene PRC1, BLM, and FANCI (because it was found to be the most prognostic amplicon in the METABRIC training data set); (vii) the breast cancer-specific ER attractor metagene consisting of genes AGR3, CA12, FOXA1, GATA3, MLPH, AGR2, ESR1, and TBC1D9; (viii) the breast cancer-specific adipocyte metagene consisting of genes ADIPOQ, ADH1B, FABP4, PLIN1, RBP4, PLIN4, G0S2, GPD1, CD36, and AOC3; (ix) the breast cancer-specific HER2 metagene consisting of genes ERBB2, PGAP3, STARD3, MIEN1, GRB7, PSMD3, and GSMDB; (x) the chr7p11.2 attractor metagene consisting of genes MRPS 17, LANCL2, SEC61G, CCTA6, CHCHD2, and EGFR; (xi) the ZMYND10 metagene consisting of genes ZYMND10, LRRC48, and CASC1; and (xii) the PGR-RAI2 metagene consisting of genes PGR and RAI2 (note that both the ZMYND10 and PGR-RAI2 metagenes were protective in that their individual CIs in all breast cancer data sets were less than 0.5). The rationale for considering certain of these particular metagenes is that additional protective features were desired, and the ones selected were highly protective and at the same time not positively correlated with the most protective feature, the FGD3-SUSD3 metagene.

Each metagene feature used in the model was defined by the average expression value of each of the 10 top-ranked genes in each attractor metagene. If, however, some of these 10 genes had mutual information with the metagene—as defined in (Cheng et al., PLoS Comput. Biol. 9, e1002920 (2013))—that was less than 0.5, it was removed from consideration when deriving the metagene feature. If a gene was profiled by multiple probes—a collection of micrometer beads that bind a specific nucleic acid sequence—the probe with the highest degree of coexpression with the metagene was selected. The selection was done by applying the iterative attractor-finding algorithm disclosed herein on all the probes for the top 10 genes and selecting the top-ranked probe for each gene. The expression values of each metagene feature were median-centered by subtracting their median value.

All the categorical—nonnumerical, such as histological type—variables in the clinical data were binarized by representing each category by a binary variable. In that case, missing values were assigned zero in each binary variable. For example, the categorical variable ER_IHC_status (a variable that describes the immunochemistry status of ER) was binarized into two binary variables: ER-positive (ER.P) and ER-negative (ER.N). ER-positive patients were assigned [1, 0] for these two variables, ER-negative patients were assigned [0, 1], and patients with missing ER status were uniquely assigned [0, 0]. Missing values in numerical variables were imputed by the average of the nonmissing values across all samples.

5.2.2.3. Conditioning of Metagene Features

Three conditioned metagene features were used in the model: the MES feature conditioned on tumor sizes of less than 30 mm and no positive lymph nodes, the LYM feature conditioned on ER-negative patients, and the LYM feature conditioned on patients with more than three positive lymph nodes. The features were conditioned by median-centering the metagene's expression values of the subgroup of samples, satisfying the condition using the subgroup's median, and setting the values of the remaining samples to zero.

5.2.2.4. Training Submodels and Making Predictions

A prognostic model selects particular features out of the set of derived features and combines them using an algorithm for optimally fitting the given survival information. The ensemble model consisted of several such submodels. The choice of these models, described below, was made based on their prognostic capability.

5.2.2.5. Cox Regression Based on Akaike Information Criterion

The Cox proportional hazards model relates the effect of a unit increase in a covariate to the hazard ratio. (Andersen et al., Ann. Stat. 10, 1100-1120 (1982)). To select from derived features as covariates in the regression model, stepwise selection was performed based on Akaike Information Criterion (AIC). (Sakamoto et al., Akaike Information Criterion Statistics (D. Reidel Publishing Company, Dordrecht, 1986). In each step, the feature with the lowest AIC measure was selected. The Cox-AIC model makes predictions by computing fitted values of the given features to the regression model. AIC was used for feature selection on molecular features and clinical features separately to fit Cox proportional hazards models. The predictions made by the two separate models were combined by summation.

5.2.2.6. Generalized Boosted Regression Models

The generalized boosted regression model (GBM) adopts the exponential loss function used in the AdaBoost algorithm (Freund et al., J. Comput. Syst. Sci. 55, 119-139 (1997)) and uses Friedman's gradient descent algorithm accompanied by subsampling to improve predictive performance and reduce computational time (Friedman, Ann. Stat. 29, 1189-12320 (2001).).

GBMs were trained on molecular features and clinical features separately, as for the Cox-AIC models. Only the clinical features that were selected by the Cox-AIC model were used as input to the GBM. Fivefold cross-validation was performed to determine the best number of trees in the model. The tree depth was set to the number of significant explanatory variables in the Cox-AIC model (P<0.05 based on t test). The predicted values made by the two separated models were combined by summation.

5.2.2.7. K-Nearest Neighbor Model

A modified version of the K-nearest neighbor (KNN) model (Venables et al. Modern Applied Statistics (Springer, New York ed. 4, 2002)) was used for survival prediction in the model. Features were selected whose values defined patients' ranking with CI greater than 0.6 or less than 0.4 in the training set.

When making predictions, the Euclidean distance in the selected feature space between the patient with unknown survival and each deceased patient in the training set was calculated. The top 10% of the deceased patients with smallest distances, defined as the “nearest neighbors,” were used to make predictions. The predictions were made by taking the weighted average of the survival times of the nearest neighbors, where the weight of a neighbor was the reciprocal of the distances between the neighbor and the patient with unknown survival.

5.2.2.8. Combination of Cox Regression and GBM Applied on Empirically Selected Features.

The performance of the overall model was improved by incorporating a submodel constrained to include the four fundamental molecular features described in Results (CIN, MES constrained to a tumor size less than 30 mm with no positive lymph node, LYM constrained to ER-negative patients, and the FGD3-SUSD3 metagene) together with very few clinical features, including the number of positive lymph nodes and the age at diagnosis. The selected features were used to fit a Cox regression model and a GBM, whose predictions were combined by summation.

5.2.2.9. Combination of Predictions

The final model contained the submodels described above. The resulting predictions from Cox-AIC and GBM, as well as the reciprocal of the predicted survival time given by the KNN model, were added and the result was divided by the corresponding 5D. The same normalization was done on the predictions derived from submodel 4, described above, and the final ensemble prediction was the summation of these two.

5.2.2.10. Combination of OS- and DS-Based Predictions

The best performance was achieved when the models were trained twice, once using OS-based survival data and again using DS-based survival data, and then combining the two predictions. Therefore, the ensemble model depicted in FIG. 5 was adopted. These two sets of predictions were combined by taking the weighted average of the two. The weights were determined by maximizing the CI with OS in the training set with a heuristic optimization technique.

### 5.2.3. Results

5.2.3.1. Participation in the Challenge

The three universal attractor metagenes used to develop the final model contain genes associated with mitotic chromosomal instability (CIN), mesenchymal transition (MES), and lymphocyte-specific immune recruitment (LYM). Because cancer is thought to be characterized by a few unifying “hallmarks”, these gene signature are referred to as “bioinformatic hallmarks of cancer” that are associated with the ability of cancer cells to divide uncontrollably, to invade surrounding tissues, and, with the effort of the organism, to fight cancer with a particular immune response. In addition, the instant model makes use of another molecular feature that was identified during participation in the Challenge: a metagene whose expression is associated with good prognosis and that contains the expression values of two genes—FGD3 and SUSD3—that are genomically adjacent to each other.

The initial phases of the Challenge were based on partitioning of the rich METABRIC breast cancer data set (Curtis et al., Nature 486, 346-352 (2012)) (which includes molecular, clinical, and survival information from 1981 patients) into two subsets: a training set and a validation set. Participants' computational models were developed on the training set and evaluated on the validation set, using a real-time leaderboard to record the performance [as determined with concordance index (CI) values, defined herein] of all submitted models. During the final phase of the Challenge, participants were given access to the full set of the METABRIC data, which had been renormalized for uniformity by Sage Bionetworks using eigen probe set analysis. (Mecham, et al., Bioinformatics 26, 1308-1315 (2010)). At that time, the computational models could be trained on that full set and submitted for evaluation against a newly generated validation data set of patients, referred to as the Oslo Validation (OsloVal) data set. Therefore, the numerical values for the results that are presented here use the full METABRIC data set to maximize accuracy, whereas the computational models were developed using the originally available training data sets.

5.2.3.2. Selection of a Numerical Score for Evaluating Prognostic Models

A “CI” (Pencina et al., Stat. Med. 23, 2109-2123 (2004)) was the numerical measure used to score all Challenge submissions on the leaderboards. In this context, the CI is a score that applies to a cohort of patients (rather than an individual patient) and evaluates the similarity between the actual ranking of patients in terms of their survival and the ranking predicted by the computational model. CI measures the relative frequency of accurate pairwise predictions of survival over all pairs of patients for which such a meaningful determination can be achieved and, therefore, is a number between 0 and 1. The average CI for random predictions is 0.5. If a model achieves a CI of 0.75, then the model will correctly order the survival of two randomly chosen patients three of four times. The final model had a CI of 0.756 in the OsloVal data set.

The METABRIC data set included both disease-specific (DS) survival data, in which all reported deaths were determined to be due to breast cancer (otherwise, a patient was considered equivalent to a hypothetical still living patient with reported survival equal to the time to actual death from other causes), and overall survival (OS) data, in which all deaths are reported even though they could potentially be due to other causes. The instant work performed in the context of the Challenge used mainly DS survival-based data, and unless otherwise noted, the CI scores referring to the METABRIC data set presented herein were evaluated using DS survival data. This is because the CIs for models developed using DS survival-based data from the METABRIC data set were found to be significantly higher than those obtained when the OS survival-based data were used. Furthermore, DS survival-based modeling did not need to include age as a prognostic feature as much as OS survival-based modeling did, which suggests that OS survival-based modeling cannot predict survival using molecular features as accurately as DS survival-based modeling, and instead needed to make use of age, which is an obvious feature for predicting survival even in healthy people.

The first phases of the Challenge consisted of participants training their prognostic computational models using a subset of samples from the full METABRIC data set as a training set, whereas the remaining subset was used to test the models by evaluating the CI scores in a realtime leaderboard. The survival data and the corresponding scoring of the OsloVal data set were OS survival-based. Accordingly, the Kaplan-Meier survival curves presented herein involving OsloVal are OS survival-based.

5.2.3.3. CI Scores for Individual Genes

As a first task, the prognostic ability of the expression level of each individual gene was quantified by computing the CI between the expression levels of the gene in all patients and the survival of those patients (Table 5). Specifically, the CIs reported in Table 5 are the CIs that would be calculated if the prognostic model consisted exclusively of the expression level of only one specific gene. For example, consider the CDCA5 gene (listed at the top of the left-hand column of Table 5). If all patients were ranked in terms of their CDCA5 expression levels, from highest to lowest, and then all patients were ranked in terms of their survival times, from shortest to longest, these two rankings would yield a CI of 0.651. This means that if two patients were randomly selected from the METABRIC data set, the one whose expression of CDCA5 is higher will have the shorter survival time 65.1% of the time. Because CDCA5 expression is associated with poor prognosis (that is, the higher the expression, the shorter the survival), CDCA5 is referred to as a poor survival—inducing gene (or simply, an “inducing gene,” which is one that displays a CI that is significantly greater than 0.5).

At the opposite end of the spectrum was the FGD3 gene, which had a CI of 0.352 (Table 5, right-hand column). This CI indicates that if one randomly chooses two patients from the METABRIC data set, then the one with lower FGD3 expression levels will have the shorter survival time 64.8% (100% minus 35.2%) of the time. Because high levels of FGD3 expression were associated with a good prognosis (that is, the higher the expression, the longer the survival), FGD3 is referred to as a survival-protective gene (or simply, a “protective” gene, which is one that displays a CI that is significantly less than 0.5). Table 5 shows two expanded lists of ranked genes: one with the most inducing genes (those with the highest CIs) and one with the most protective genes (those with the lowest CIs).

In the following, all references to gene expression levels, including average values and numbers on scatter-plot axes, are assumed to be log 2-normalized. For each attractor metagene, when the top-ranked genes are referred to, it refers to those that had the highest mutual information with the attractor metagene, as previously described (Cheng et al., PLoS Comput. Biol. 9, e1002920 (2013)).

5.2.3.4. Mitotic CIN Attractor Metagene

In the Challenge, the mitotic CIN attractor metagene was represented with the average of the expression levels of the 10 top-ranked genes from the previously evaluated (Cheng et al., PLoS Comput. Biol. 9, e1002920 (2013)) attractor metagene: CENPA, DLGAP5, MELK, BUB1, KIF2C, KIF20A, KIF4A, CCNA2, CCNB, and NCAPG. The metagene defined by this average is referred to as the “CIN feature.” It contains many genes that encode proteins that are part of the kinetochore—a structure at which spindle fibers attach during cell division to segregate sister chromatids—particularly those involved in the microtubule-kinetochore interface, suggesting a biological mechanism by which mitotic chromosomal instability in dividing cancer cells gives rise to daughter cells with genomic modifications, some of which pass the test of natural selection. The mitotic CIN attractor metagene has previously been shown to be strongly associated with tumor grade (a classification system that measures how abnormal a cancer cell appears when assessed microscopically) in multiple cancers (Cheng et al., PLoS Comput. Biol. 9, e1002920 (2013)).

The mitotic CIN attractor metagene was essentially rediscovered by identifying the genes for which expression was most associated with poor prognosis in the METABRIC data set. Indeed, all 10 genes (listed above) of the CIN feature that were used in the Challenge were among the 50 genes listed in the left column of Table 5; furthermore, 40 of the 50 genes listed in the left column of Table 5 were among the top 100 genes of the CIN attractor metagene identified previously (Cheng et al., PLoS Comput. Biol. 9, e1002920 (2013)) (the P value for such overlap is less than 1.04×10⁻⁹⁷ based on Fisher's exact test).

As outlined in Table 5, individual genes were ranked in terms of their CIs with respect to gene expression and survival data in the METABRIC data set. The CI measures the similarity of patient rankings based on the expression level of the gene compared to the actual rankings based on DS survival data. Shown on the left are the most “inducing” genes with the highest CIs. Shown on the right are the most protective genes with the lowest CIs. The underlined genes are among the top 100 genes of the CIN attractor metagene defined in (Cheng et al., PLoS Comput. Biol. 9, e1002920 (2013)). The probe IDs are identifiers for probes designed by Illumina. If a gene was profiled by multiple probes, the probe with the highest difference from the average CI for random predictions, 0.5, was chosen. Genes identified by asterisks are among the 10 top-ranked genes of the CIN attractor metagene and were used in the model

TABLE 5
CIN expression and survival.

| | Gene | Concordance | | Gene | Concordance |
|---|---|---|---|---|---|
| Probe ID | Symbol | Index | Probe ID | Symbol | Index |
| ILMN_1683450 | CDCA5 | 0.651 | ILMN_1772686 | FGD3 | 0.352 |
| ILMN_1714730 | UBE2C | 0.644 | ILMN_1785570 | SUSD3 | 0.358 |
| ILMN_1801939 | CCNB2* | 0.643 | ILMN_2310814 | MAPT | 0.372 |
| ILMN_1700337 | TROAP | 0.643 | ILMN_2353862 | LRRC48 | 0.374 |
| ILMN_2357438 | AURKA | 0.642 | ILMN_2397954 | PARP3 | 0.374 |
| ILMN_1781943 | FAM83D | 0.640 | ILMN_1674661 | CIRBP | 0.375 |
| ILMN_2212909 | MELK* | 0.640 | ILMN_1801119 | BCL2 | 0.376 |
| ILMN_1695658 | KIF20A* | 0.639 | ILMN_1708983 | CASC1 | 0.377 |
| ILMN_1673721 | EXO1 | 0.639 | ILMN_1772588 | CCDC170 | 0.377 |
| ILMN_1786125 | CCNA2* | 0.638 | ILMN_1849013 | HS.570988 | 0.378 |
| ILMN_1801257 | CENPA* | 0.638 | ILMN_1809639 | TMEM26 | 0.378 |
| ILMN_1796949 | TPX2 | 0.637 | ILMN_1657361 | CBX7 | 0.380 |
| ILMN_1771039 | GTSE1 | 0.637 | ILMN_1713162 | GSTM2 | 0.380 |
| ILMN_1716279 | CENPE | 0.637 | ILMN_1806456 | C14orf45 | 0.380 |
| ILMN_1808071 | KIF14 | 0.636 | ILMN_1790315 | C7orf63 | 0.381 |
| ILMN_2077550 | RACGAP1 | 0.636 | ILMN_1667716 | TMEM101 | 0.382 |
| ILMN_1736176 | PLK1 | 0.636 | ILMN_1907649 | HS.144312 | 0.382 |
| ILMN_1703906 | HJURP | 0.636 | ILMN_1811014 | PGR | 0.382 |
| ILMN_1663390 | CDC20 | 0.636 | ILMN_1807211 | NICN1 | 0.382 |
| ILMN_1751776 | CKAP2L | 0.635 | ILMN_1805104 | ABAT | 0.382 |
| ILMN_2344971 | FOXM1 | 0.635 | ILMN_1655117 | WDR19 | 0.383 |
| ILMN_1751444 | NCAPG* | 0.635 | ILMN_1696254 | CYB5D2 | 0.383 |
| ILMN_1747016 | CEP55 | 0.634 | ILMN_1777342 | PREX1 | 0.383 |
| ILMN_2042771 | PTTG1 | 0.634 | ILMN_2183692 | PHYHD1 | 0.384 |
| ILMN_1740291 | POLQ | 0.633 | ILMN_2128795 | LRIG1 | 0.384 |
| ILMN_2202948 | BUB1* | 0.633 | ILMN_1784783 | NME5 | 0.384 |
| ILMN_1685916 | KIF2C* | 0.633 | ILMN_1862217 | HS.532698 | 0.384 |
| ILMN_2413898 | MCM10 | 0.632 | ILMN_1815705 | LZTFL1 | 0.384 |
| ILMN_1713952 | Clorfl06 | 0.632 | ILMN_1670925 | CYB5D1 | 0.385 |
| ILMN_1684217 | AURKB | 0.632 | ILMN_1684034 | STAT5B | 0.386 |
| ILMN_1815184 | ASPM | 0.632 | ILMN_1664922 | FLNB | 0.387 |
| ILMN_1737728 | CDCA3 | 0.632 | ILMN_1794213 | ABHD14A | 0.387 |
| ILMN_1702197 | SAPCD2 | 0.630 | ILMN_1776967 | DNAAF1 | 0.387 |
| ILMN_1728934 | PRC1 | 0.630 | ILMN_1736184 | GSTM3 | 0.387 |
| ILMN_1739645 | ANLN | 0.629 | ILMN_1760574 | RAI2 | 0.387 |
| ILMN_2049021 | PTTG3 | 0.629 | ILMN_2341254 | STARD13 | 0.387 |
| ILMN_1670238 | CDC45 | 0.628 | ILMN_1651364 | PCBD2 | 0.387 |
| ILMN_1799667 | KIF4A* | 0.628 | ILMN_1769382 | KBTBD3 | 0.387 |
| ILMN_1788166 | TTK | 0.628 | ILMN_1697317 | DYNLRB2 | 0.387 |
| ILMN_1771734 | GMPSP1 | 0.627 | ILMN_1790350 | TPRG1 | 0.388 |
| ILMN_1811472 | KIF23 | 0.627 | ILMN_1664348 | PNPLA4 | 0.389 |
| ILMN_1666305 | CDKN3 | 0.627 | ILMN_2125763 | ZMYND10 | 0.389 |
| ILMN_1731070 | ORC6 | 0.627 | ILMN_2323385 | TRIM4 | 0.389 |
| ILMN_2413650 | STIL | 0.626 | ILMN_1657451 | SRPK2 | 0.389 |
| ILMN_1770678 | CBX2 | 0.626 | ILMN_1779416 | SCUBE2 | 0.390 |
| ILMN_1749829 | DLGAP5* | 0.625 | ILMN_1719622 | RABEP1 | 0.391 |
| ILMN_1789510 | STIP1 | 0.624 | ILMN_1687351 | ANKRA2 | 0.391 |
| ILMN_1814281 | SPC25 | 0.624 | ILMN_1691884 | STC2 | 0.391 |
| ILMN_1709294 | CDCA8 | 0.624 | ILMN_2140700 | CRIPAK | 0.393 |
| ILMN_1671906 | MND1 | 0.624 | ILMN_1858599 | HS.20255 | 0.393 |

The results regarding this and other attractor metagenes were validated in a statistically significant manner in the OsloVal data set despite its relatively small size (184 samples). For example, FIG. 2 shows the Kaplan-Meier cumulative survival curves of the CIN feature for the METABRIC (P<2×10⁻¹⁶ using log-rank test) and OsloVal (P=0.0041 using log-rank test) data sets, comparing tumors with high and low values of the CIN feature. These data confirmed that poor prognosis was associated with expression of the mitotic CIN attractor metagene.

5.2.3.5. MES Attractor Metagene

In the Challenge, the MES attractor metagene was represented with the average of the expression levels of the 10 top-ranked genes from the previously evaluated (Cheng et al., PLoS Comput. Biol. 9, e1002920 (2013)) attractor metagene: COL5A2, VCAN, SPARC, THBS2, FBN1, COL1A2, COL5A1, FAP, AEBP1, and CTSK. The metagene defined by this average is referred to as the MES feature. A nearly identical signature had been previously identified (Kim et al., BMC Med. Genomics 3, 51 (2010)) from its association with tumor stage (a measure of the extent to which the cancer has spread to adjacent lymph nodes or distant sites in the body). Specifically, the signature is expressed in high amounts only in tumor samples from patients whose cancer has exceeded a defined stage threshold, which is cancer type-specific. For example, in breast cancer, the MES signature appears early, when in situ carcinoma becomes invasive (stage I); in colon cancer, it is expressed when stage II is reached; and in ovarian cancer, it is expressed when stage III is reached. Identification of stage-specific differentially expressed genes in these three cancers reveals strong enrichment of the signature. This differential expression results from the fact that the signature is present in some, but not all, samples in which the stage threshold is exceeded, but never in samples in which the stage threshold has not been reached. That is, the presence of the signature implies tumor invasiveness, but its absence is uninformative.

Related versions of the MES signature were found to be prognostic in various cancers, such as oral squamous cell carcinoma (Cheng et al., PLoS Comput. Biol. 9, e1002920 (2013)) and ovarian cancer (Tothill et al., Clin. Cancer Res. 14, 5198-5208 (2008)). In breast cancer, however, the prognostic ability of the MES feature individually was not significant. This lack of prognostic power may be explained by the fact that the presence of the MES signature in breast cancer implies that the tumor is invasive, but this was the case anyway for nearly all patients in the METABRIC data set.

Therefore, the MES signature was considered to be potentially prognostic only for very early stage breast cancer patients, which was defined by the absence of positive lymph nodes combined with a tumor size less than 30 mm. This restriction improved prognostic ability, however it still did not reach the level of statistical significance. However, when used in combination with the other features, this restricted version of the MES signature was helpful toward the performance of the final model. This was confirmed, as described below, by the fact that the prognostic power of the final model was reduced when eliminating the MES feature.

5.2.3.6. LYM Attractor Metagene

In the Challenge, the LYM attractor metagene was represented with the average of the expression levels of the 10 top-ranked genes from the previously evaluated (Cheng et al., PLoS Comput. Biol. 9, e1002920 (2013)) attractor metagene: PTPRC (CD45), CD53, LCP2 (SLP-76), LAPTM5, DOCK2, IL10RA, CYBB, CD48, ITGB2 (LFA-1), and EVI2B. The metagene defined by this average is referred to as the LYM feature. The composition of this gene signature indicates that a signaling pathway that includes the protein tyrosine phosphatase receptor type C (also called CD45; encoded by PTPRC) and leukocyte surface antigen CD53 has a role in patient survival. The top-ranked genes in the LYM attractor metagene, including ADAP (FYB), are known to participate in a particular type of immune response in which the LFA-1 integrin mediates costimulation of T lymphocytes that are regulated by the SLP-76-ADAP adaptor molecule, because all the corresponding genes, including ADAP (FYB), were among the top-ranked genes of the LYM attractor metagene.

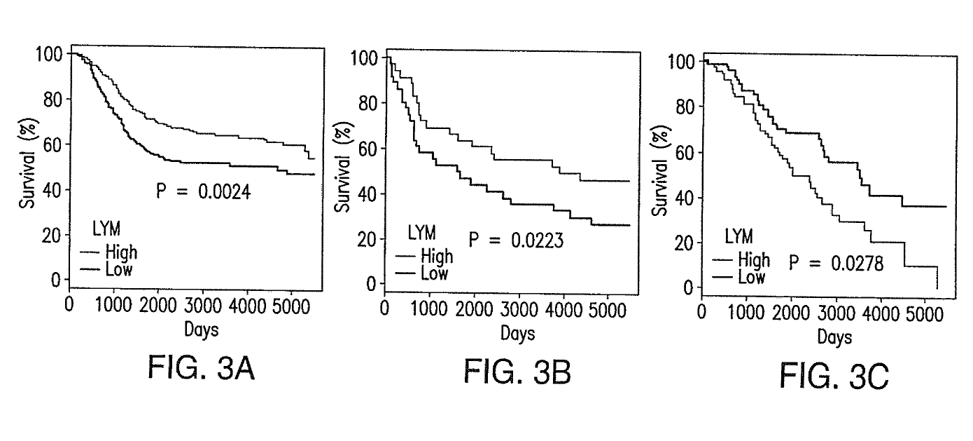

By itself, the LYM feature was slightly protective (CI<0.5) in the METABRIC data set but was not significantly associated with prognosis. Therefore, the prognostic power of the feature was tested on various subsets of patients grouped on the basis of histology, estrogen receptor (ER) status, etc. The LYM feature was strongly protective in ER-negative breast cancer in the METABRIC data set, and this observation was validated in the OsloVal data set; FIG. 3A shows Kaplan-Meier survival curves for ER-negative patients from the METABRIC data set (P=0.0024 using log-rank test); FIG. 3B shows Kaplan-Meier survival curves for ER-negative patients from the OsloVal data set (P=0.0223 using log-rank test). In both cases, the curves compare tumors with high and low values of the LYM feature.

By contrast, the effect on prognosis was reversed for patients who had ER-positive cancers and multiple cancer cell-positive lymph nodes; FIG. 3C shows the Kaplan-Meier survival curves for METABRIC patients with ER-positive status and more than four positive lymph nodes, comparing tumors with high and low values of the LYM feature (P=0.0278 using log-rank test). There were only 19 corresponding samples in the OsloVal data set, insufficient for validation of this reversal.

5.2.3.7. FGD3-SUSD3 Metagene

As shown in Table 5, the FGD3 and SUSD3 genes were found to be the most protective ones in the METABRIC data set, with CIs equal to 0.352 and 0.358, respectively. Therefore, these were considered to be promising candidates to be included as features in the prognostic model. The two genes are genomically adjacent to each other at chromosome 9q22.31. In the final prognostic model, a FGD3-SUSD3 metagene was used, which was defined by the average of the two expression values.

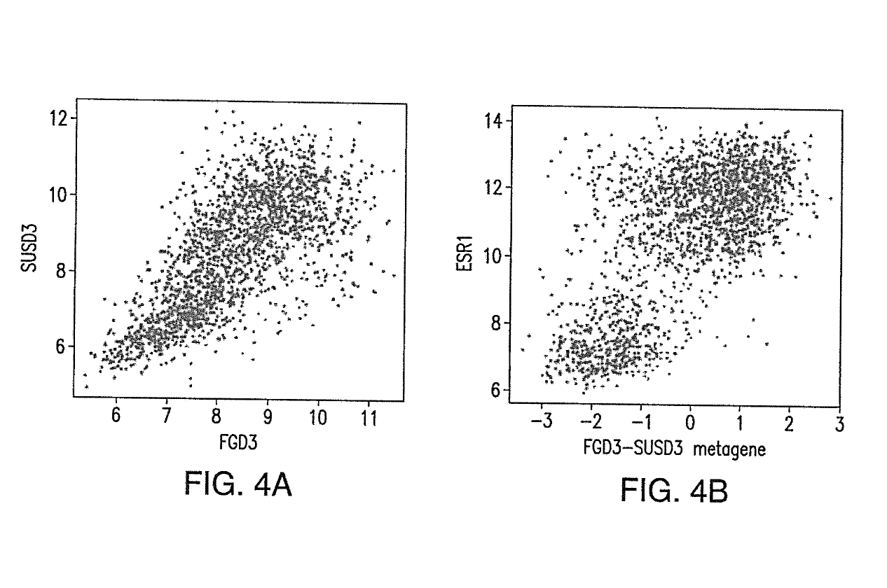

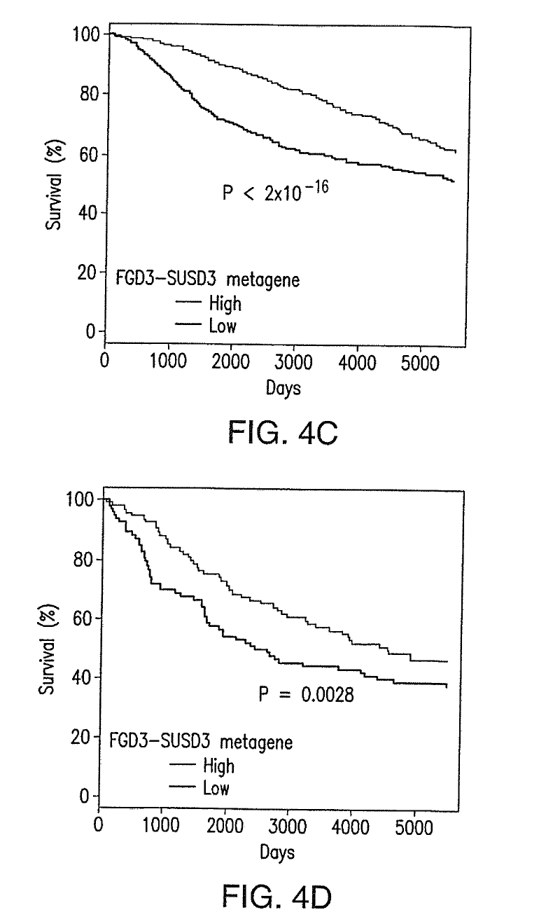

A scatter plot (FIG. 4A) of the METABRIC expression levels of FGD3 versus SUSD3 showed that the two genes did not appear to be coregulated when one or the other gene was highly expressed, but the genes did appear to be simultaneously silent (that is, low expression of one gene implies low expression of the other). The CIs for the FGD3-SUSD3 metagene and the estrogen receptor 1 (ESR1) gene in the METABRIC data set were 0.346 and 0.403, respectively, indicating that the lack of FGD3-SUSD3 expression was more strongly associated with poor prognosis compared with lack of expression of ESR1. Furthermore, a scatter plot (FIG. 4B) of the METABRIC expression levels of the FGD3-SUSD3 metagene versus ESR1 revealed that the two features were associated in the sense that ER negative breast cancers tended to express low levels of the FGD3-SUSD3 metagene, but the reverse was not necessarily true.

The poor prognosis associated with low expression of the FGD3-SUSD3 metagene was validated in the OsloVal data set. FIG. 4C shows the Kaplan-Meier curves for the FGD3-SUSD3 metagene in the METABRIC data set (P<2×10⁻¹⁶ using log-rank test). FIG. 4D shows the Kaplan-Meier survival curves for the FGD3-SUSD3 metagene in the OsloVal data set (P=0.0028 using log-rank test). In both cases, the curves compare tumors with high and low expression of the FGD3-SUSD3 metagene.

5.2.3.8. Breast Cancer Prognosis Challenge Model

The development of the breast cancer prognosis model for the Challenge is described in detail in Materials and Methods section, above. It used, as potential features, several metagenes that had been identified previously (Cheng et al., PLoS Comput. Biol. 9, e1002920 (2013)), the FGD3-SUSD3 metagene, and certain clinical phenotypes. During the course of the Challenge, several combinations of prognostic algorithms (based on various statistical and machine-learning techniques) were tested, each of which defined a computational model that automatically selected some of the potential features and achieved prediction of survival. These are referred to herein as “submodels,” which were eventually combined into one “ensemble” model.

FIG. 5 shows the Kaplan-Meier cumulative survival curves for the final ensemble prognostic model using the OsloVal data set (the P value derived from the log-rank test was lower than the minimum computable one, which was 2×10⁻¹⁶ using log-rank test), comparing patients with “poor” and “good” predicted survival according to the ranking assigned by the model, which was trained on the METABRIC data set.

The corresponding CI of the final ensemble model in the OsloVal data set was 0.7562. To test whether three of the features—CIN, MES, and LYM—contributed toward increasing the CI for the model using the OsloVal data set, the CIs were evaluated after removing each feature separately and retraining the model on the METABRIC data set without it. The resulting CI after removing the CIN feature and keeping the MES and LYM features was 0.7526, the CI after removing the MES feature and keeping the CIN and LYM features was 0.7514, and the CI after removing the LYM feature and keeping the CIN and MES features was 0.7488. In all cases, the CI was lower than that of the ensemble model. These results are consistent with each of these three attractor metagenes providing information useful for breast cancer prognosis.

5.2.3.9. Comparison with Random Gene Expression Signatures

Venet et al. recently observed that randomly chosen gene expression signatures may often be significantly associated with breast cancer outcome. (Venet et al., PLoS Comput. Biol. 7, e1002240 (2011)). To explain this phenomenon, the authors introduced a specially defined proliferation signature—called meta-PCNA—which consists of 127 genes whose expression levels were most positively correlated with that of the proliferation marker PCNA, as determined from a gene expression data set of normal tissues. They observed that the meta-PCNA signature, although derived from an analysis of normal tissues, was prognostic for breast cancer outcome, and that the expression levels of many other genes were also associated with the meta-PCNA signature to varying degrees. Thus, they explained the observed association of random signatures with breast cancer outcome by the fact that several member genes of such random signatures are likely to be associated with those prognostic genes.

The meta-PCNA signature is highly similar to the mitotic CIN attractor metagene described herein. Indeed, 39 of the 127 genes in the meta-PCNA signature are among the 100 top-ranked genes of the CIN attractor metagene (Cheng et al., PLoS Comput. Biol. 9, e1002920 (2013)) (the P value for such overlap is 1.07×10⁻⁵⁴ based on Fisher's exact test). Furthermore, 7 of the 10 genes (CENPA, MELK, KIF2C, KIF20A, KIF4A, CCNA2, and CCNB2) of the CIN feature used in the Challenge are among the 127 genes of the meta-PCNA signature.

Therefore, both the meta-PCNA signature, which was derived from normal tissue analysis, and the mitotic CIN attractor metagene, which was derived from a multicancer analysis, can be used to explain the observed phenomenon that random gene expression signatures are associated with breast cancer outcome. To compare the mitotic CIN attractor metagene with the meta-PCNA signature, the corresponding CIs were evaluated for the two breast cancer data sets (NKI and Loi) used in the meta-PCNA study, for the METABRIC data set using both DS- and OS-based survival data, and for the OsloVal data set. In all five cases, the CIs of the CIN feature were slightly higher than those of the meta-PCNA signature (Table 2). This can be explained if the large “mitotic” component of the mitotic CIN attractor metagene is not considered exclusively cancer-associated, as it is also found in normal cells. By contrast, the “chromosomal instability” component of the mitotic CIN attractor metagene can be cancer-related and can account for the observed slightly higher association with survival compared with the meta-PCNA signature. Furthermore, the performance of the ensemble model with the OsloVal data set was higher than that of the CIN metagene alone.

### 5.2.4. Discussion

Even though features discovered previously from an unsupervised and multicancer analysis were used without using the METABRIC data set for training, the model described herein proved highly predictive of survival in breast cancer within the context of the Challenge. Therefore, these features appear to represent important molecular events in cancer development and can be associated with cancer-related phenotypes other than survival, such as response to drugs.

Several cancer-related gene signatures that share similarity with the mitotic CIN and MES attractor metagenes have been reported (Sotiriou et al., J. Natl. Cancer Inst. 98, 262-272 (2006); Carter et al., Nat. Genet. 38, 1043-1048 (2006); Fredlund et al., Breast Cancer Res. 14, R113 (2012); and Farmer et al., Nat. Med. 15, 68-74 (2009)). The key advantage of the attractor metagenes is that they are sharply defined by independent analyses, after being discovered separately and in nearly identical form in multiple cancer types, and can thus point to the few top ranked genes for each attractor metagene. In the short term, these select genes can be tested for their ability to improve the performance of current cancer biomarker products. Existing clinical biomarker products include some genes that are components of attractor metagene signatures but do not rank at the top of their corresponding ranked list of genes. For example, the CENPA, PRC1, and ECT2 genes are among those used in Agendia's MammaPrint breast cancer assay, and CCNB1, BIRC5, AURKA, MKI67, and MYBL2 are used in Genomic Health's Oncotype DX assay for breast cancer. All eight of these genes are included in the ranked list of the top 100 genes of the CIN attractor metagene (Cheng et al., PLoS Comput. Biol. 9, e1002920 (2013)). It would be reasonable to test whether replacing such genes with a choice that more closely represents the mitotic CIN attractor metagene would improve the accuracy of these products.

Notably absent from the selected features are copy number variations (CNVs), although such data were provided in uniformly renormalized form for both the METABRIC and OsloVal data sets. CNVs were included in earlier versions of the model and it was found that they did not improve performance in the presence (but not in the absence) of the CIN attractor metagene. Although a CNV-based “genomic instability index” (GII) was used as part of a milestone performance before the start of the Challenge, the inclusion of the CIN expression-based feature nullified the prognostic ability of GII as well as of all the individual CNVs employed in early versions of the model. Even for the amplicons, it was found that the corresponding expression-based attractor metagenes consistently had higher prognostic ability compared to any kind of CNV-based features. Therefore, it appears that (i) the components of the mitotic CIN metagene play fundamental biological roles that function upstream of biological aberrations caused by genomic alterations in cancer, and (ii) the biological effects of CNVs are more directly manifested by the expression of a few highly ranked genes in the corresponding amplicon attractor than by the presence of CNVs in the corresponding genomic region.

### 5.3. Example 3

### Identification of CIN, MES, and LYM in the PANCAN12 Data

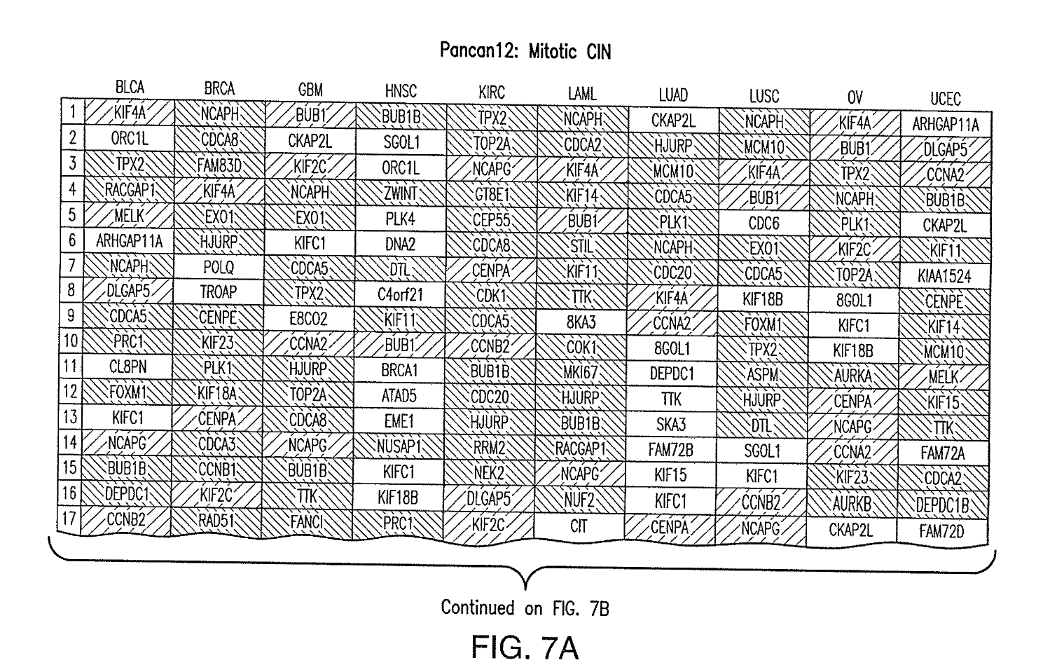

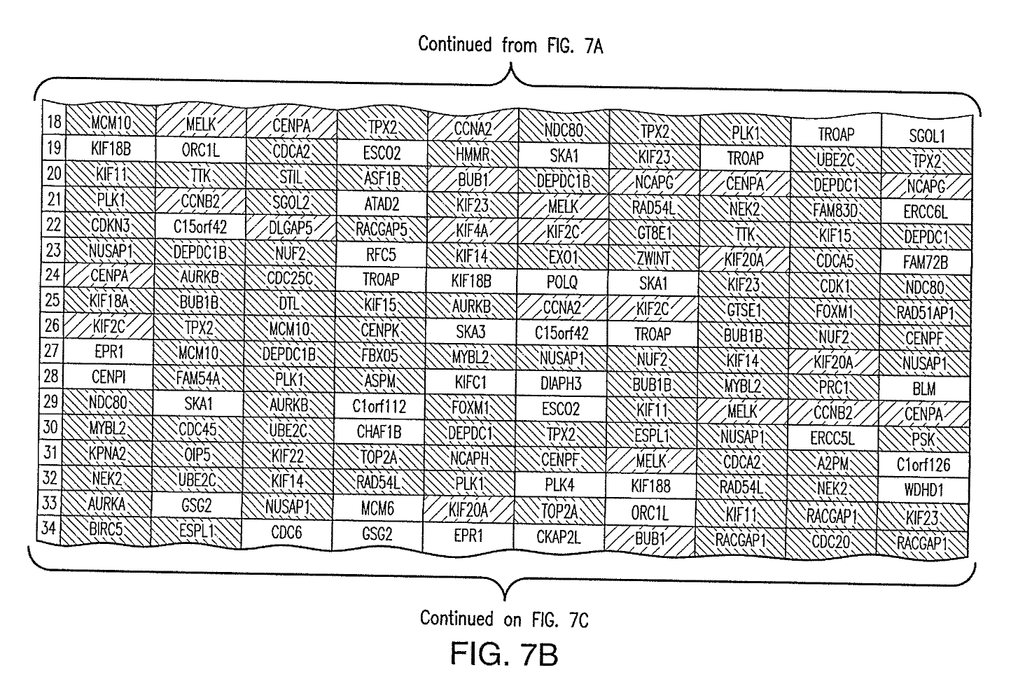

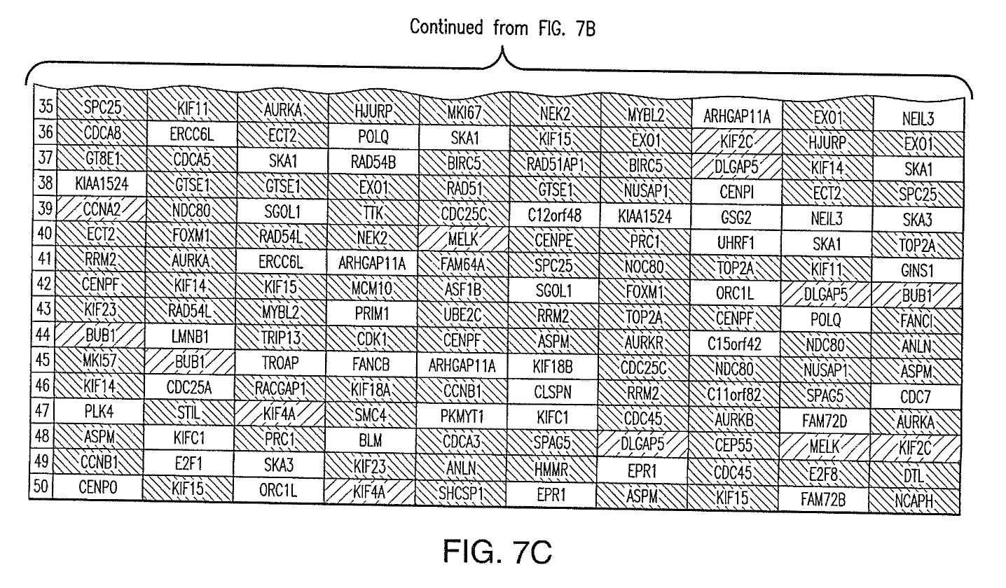

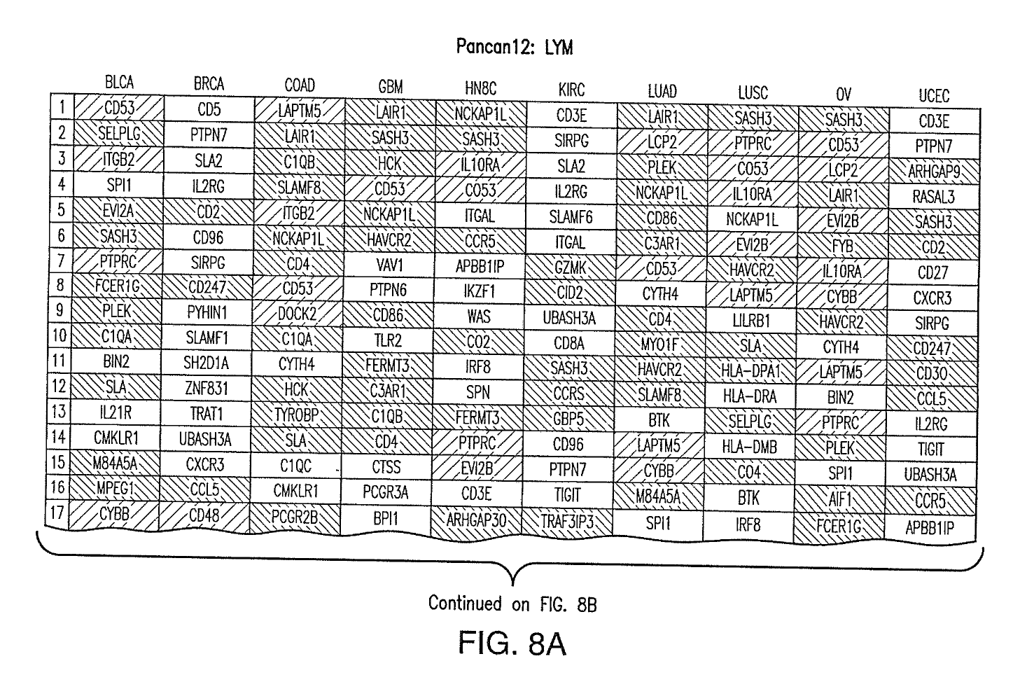

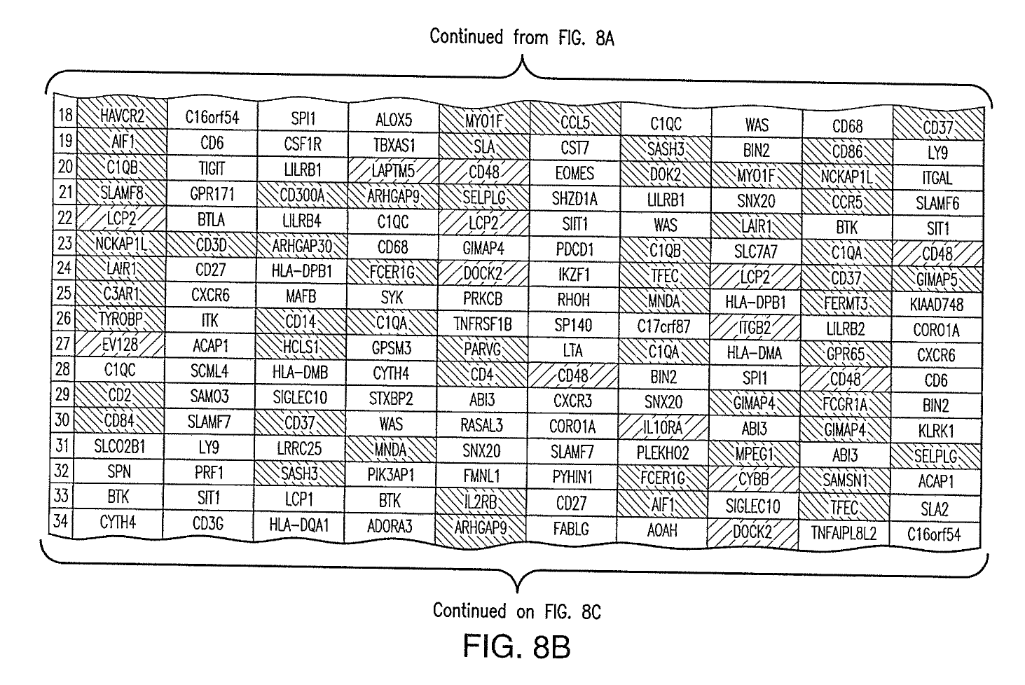

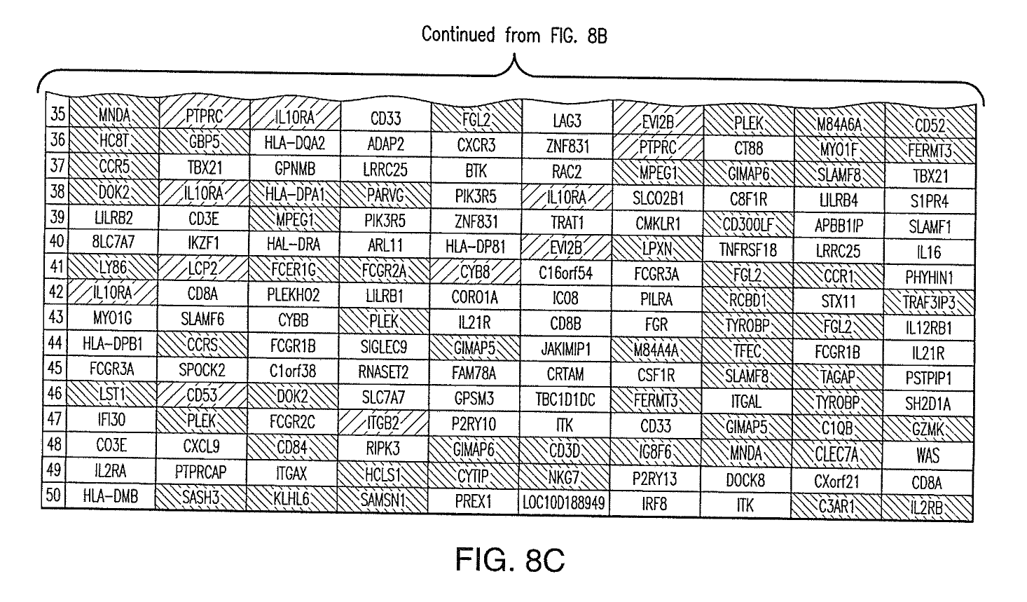

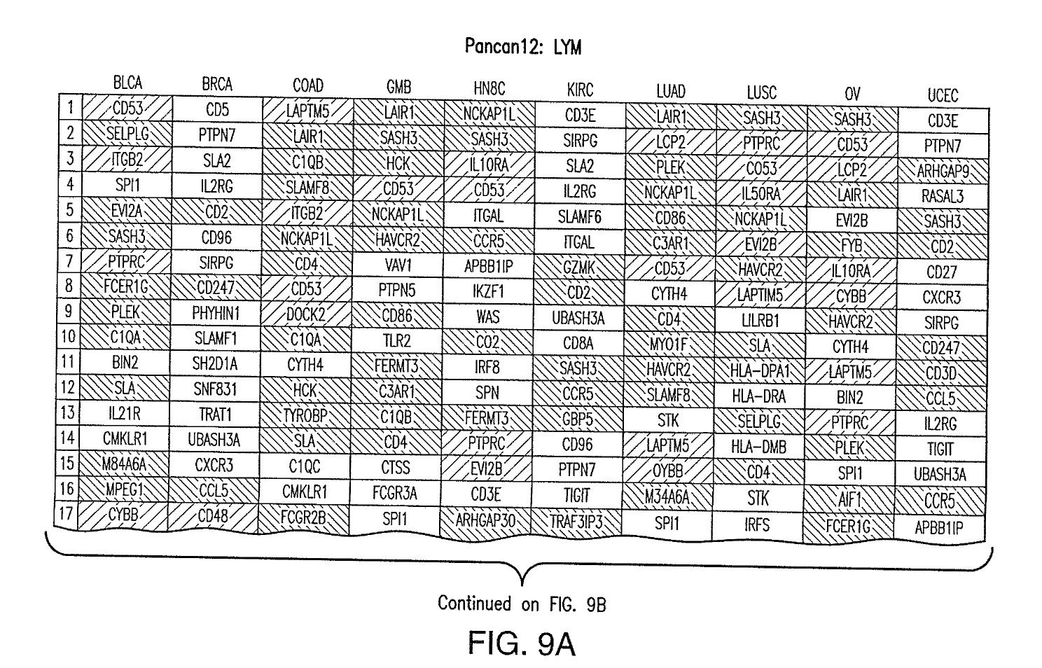

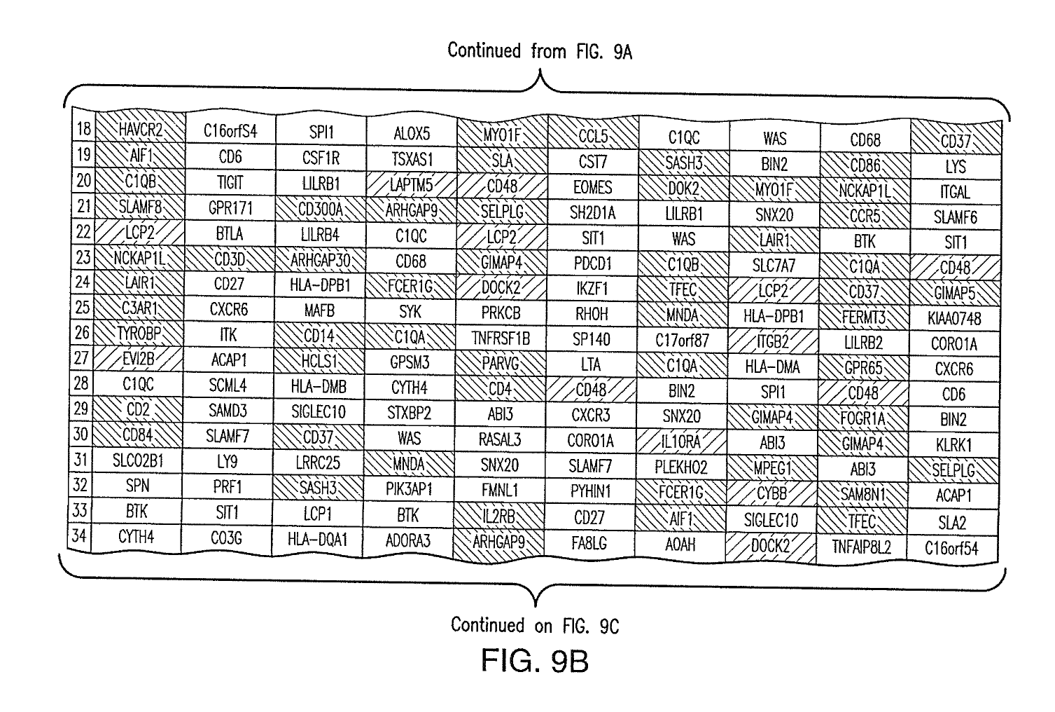

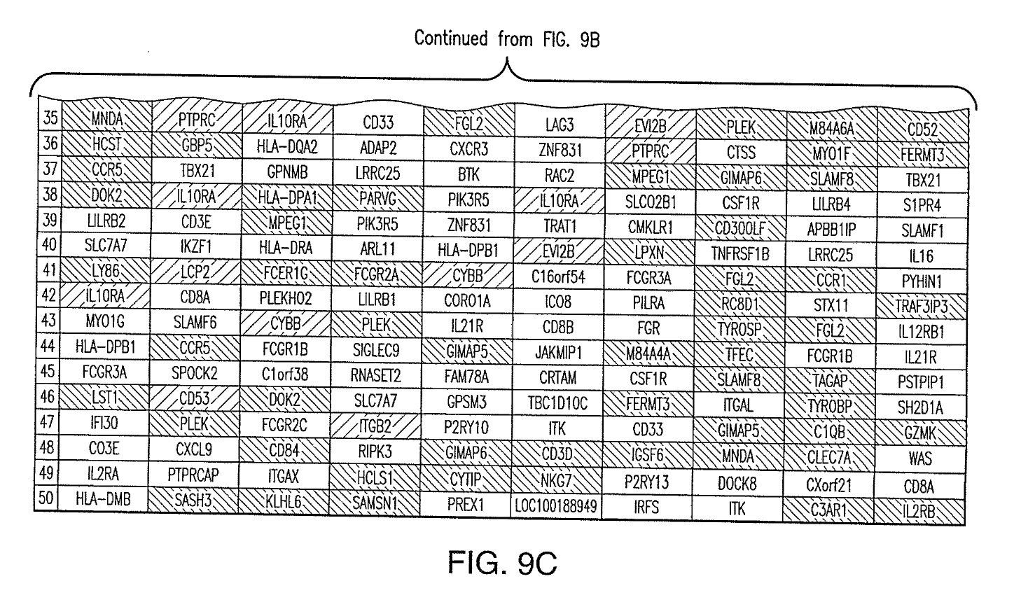

Tables 1, 2, and 3, presented above, provide lists of the top 100 genes for each of three of the attractor metagenes (CIN, MES, LYM, respectively) disclosed in the instant application. That such attractor metagenes represent phenomena occurring in different cancer types can be tested by identifying similar attractor metagenes in samples from different types of cancer. For example, by applying the algorithm outlined in Example 1 to the PANCAN12 datasets available from the Cancer Genome Atlas (a joint effort of the National Cancer Institute (NCI) and the National Human Genome Research Institute (NHGRI), two of the 27 Institutes and Centers of the National Institutes of Health, U.S. Department of Health and Human Services), these three attractor metagenes were identified in at least 10 of the 12 datasets in each case (e.g., the 71-sample READ dataset is not sufficiently rich). FIGS. 7-9 depict the corresponding attractors for the CIN, MES and LYM metagenes in the PANCAN12 data. Highlighted (light shading for top 10, dark shading for remaining 90) are the genes from Tables 1, 2, and 3, that appear in the PANCAN12 data, demonstrating huge enrichment and validating the results disclosed herein.

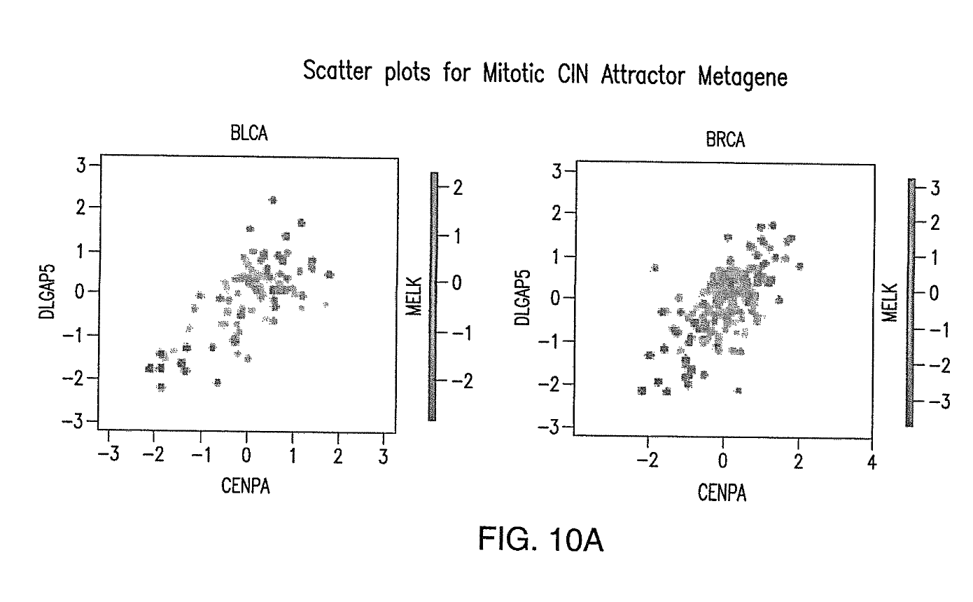

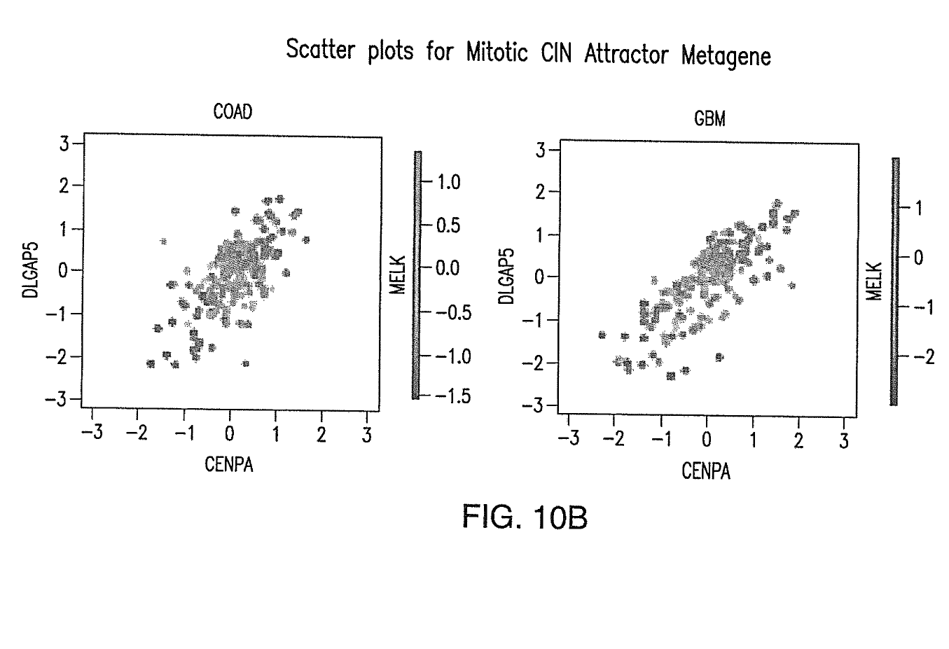

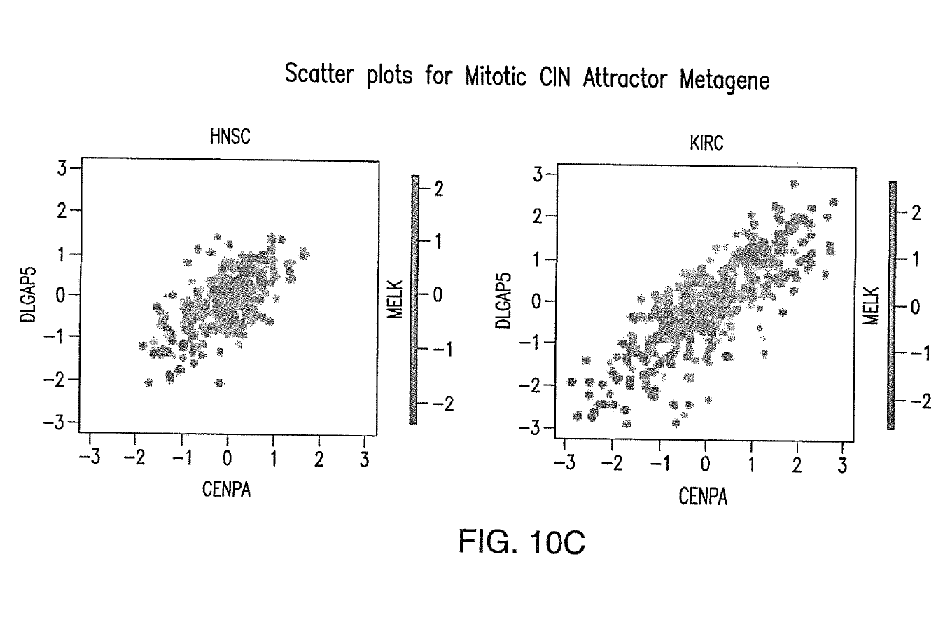

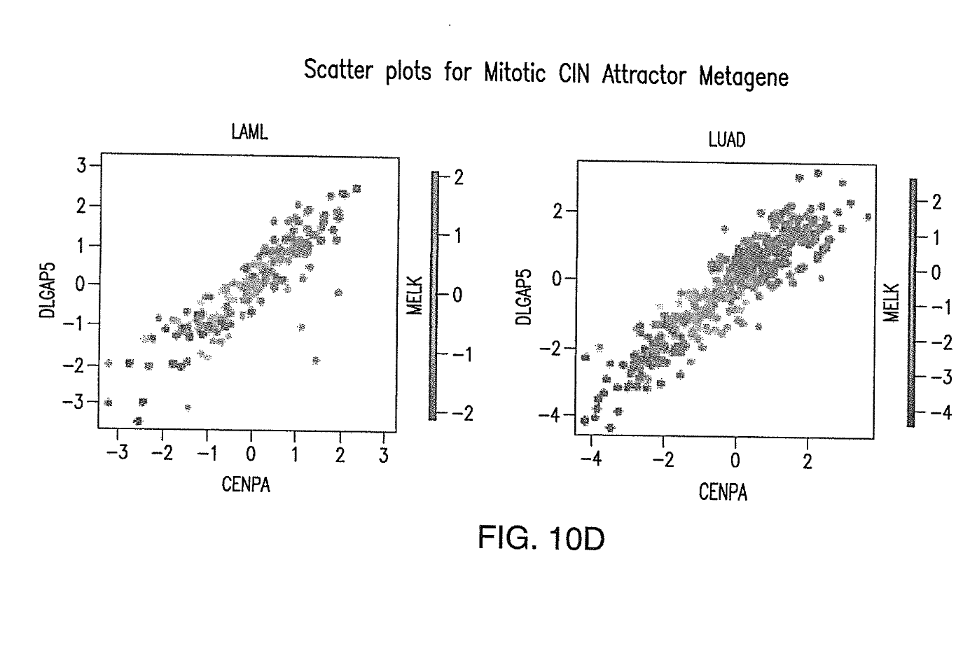

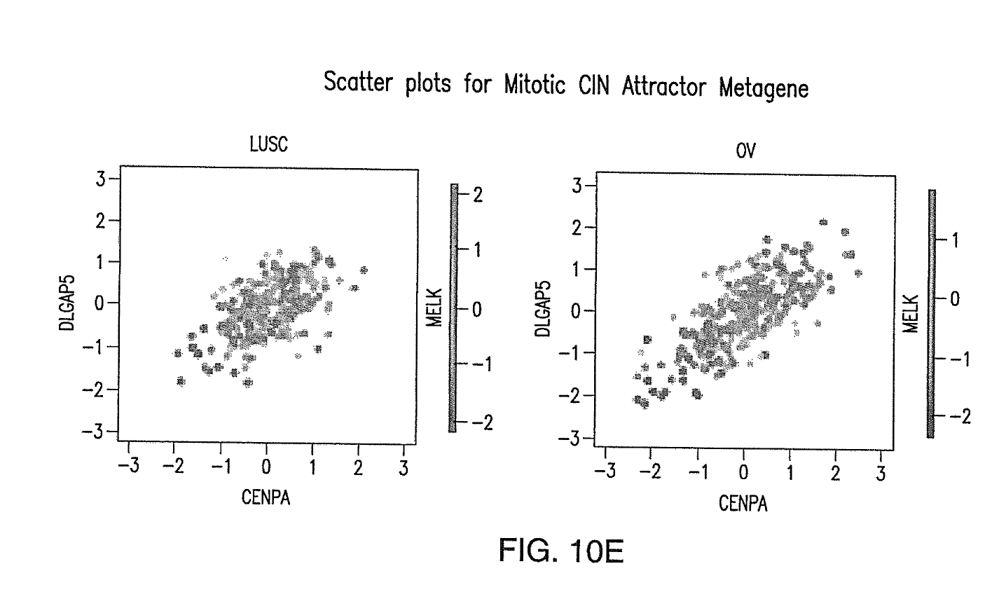

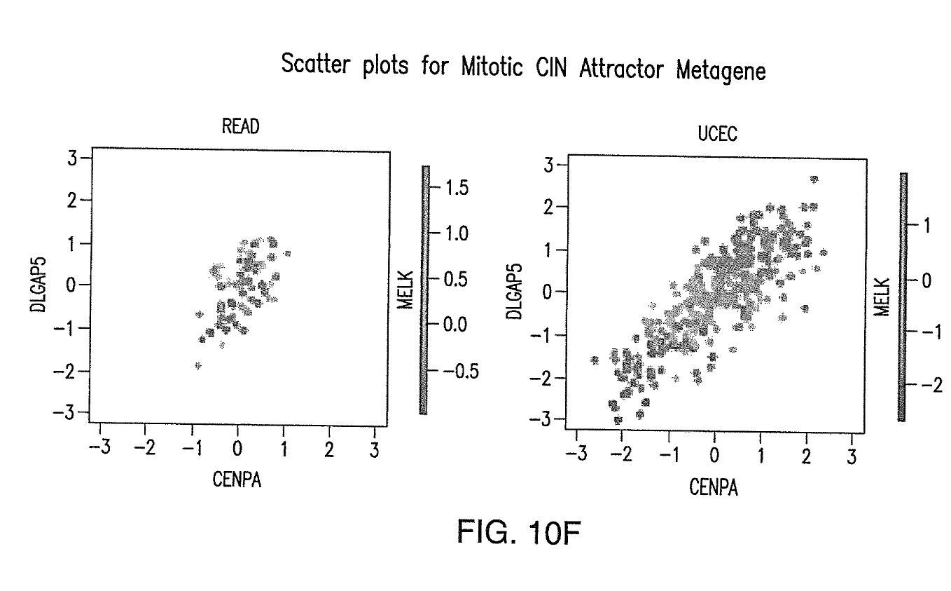

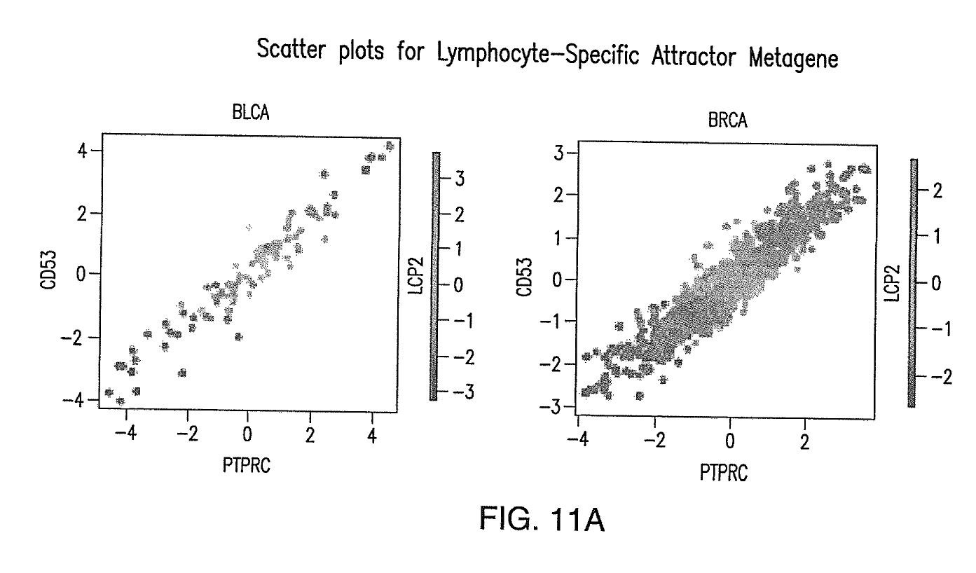

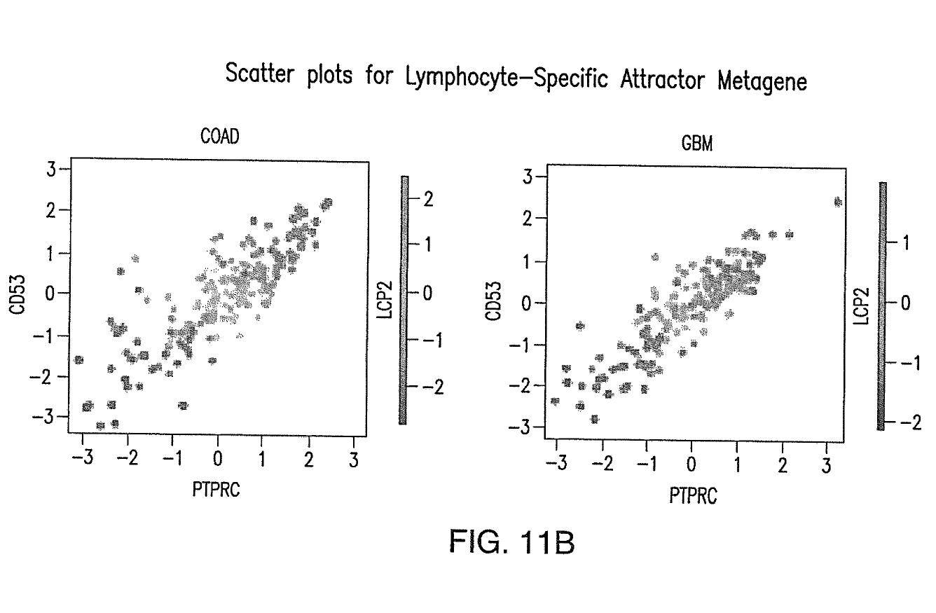

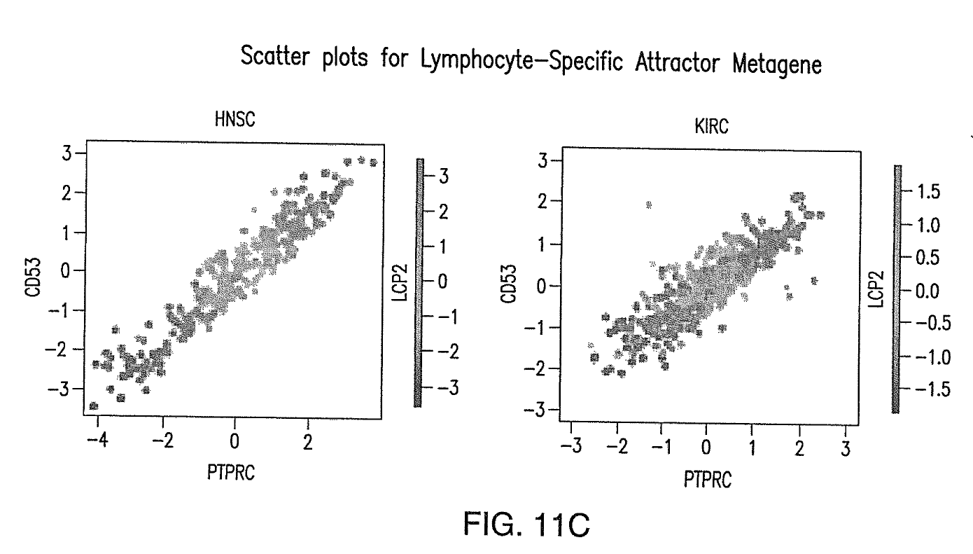

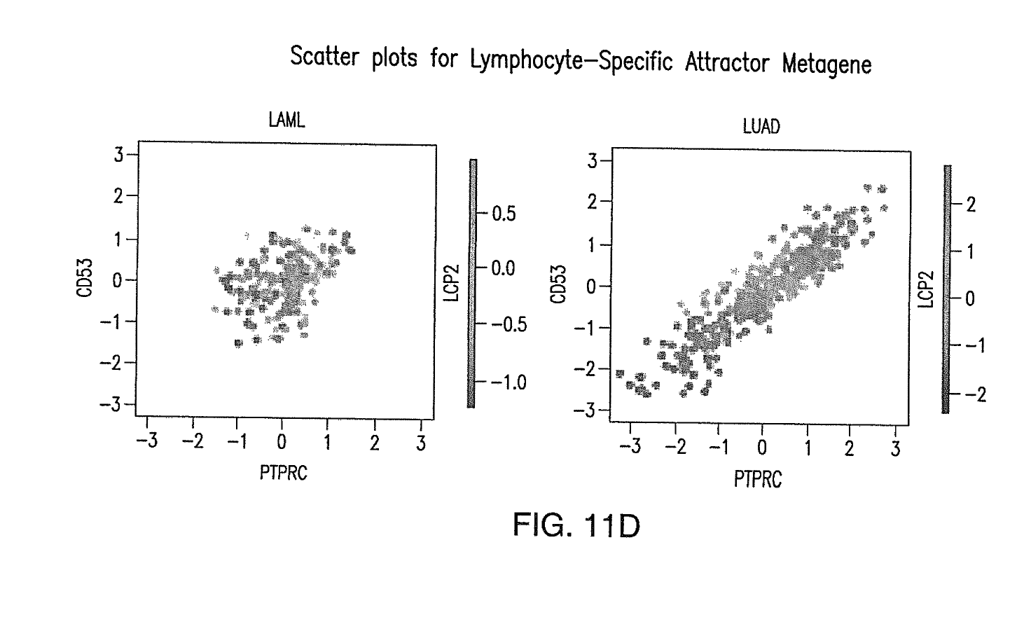

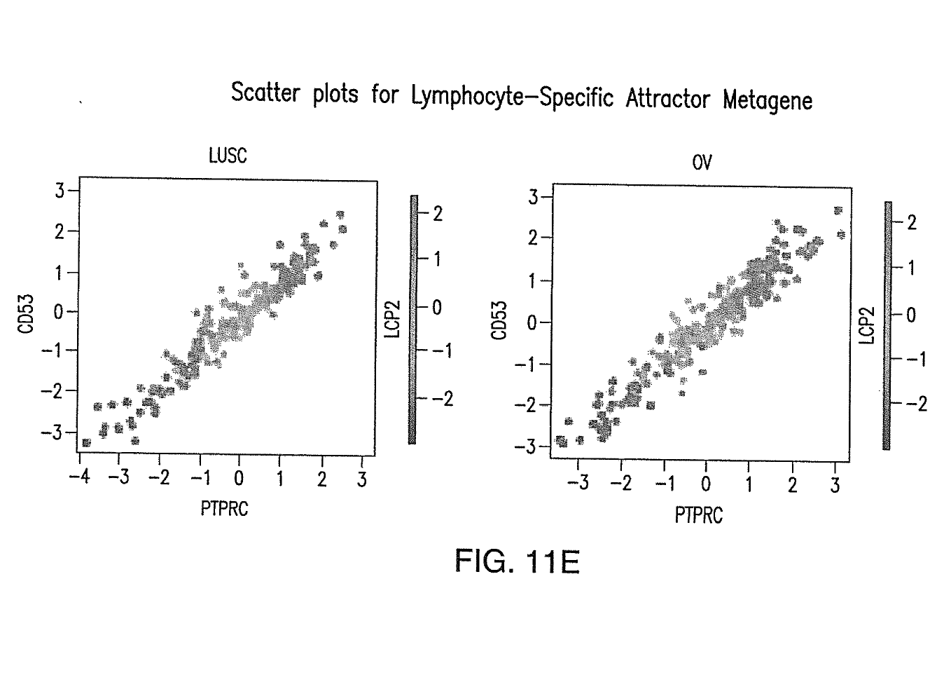

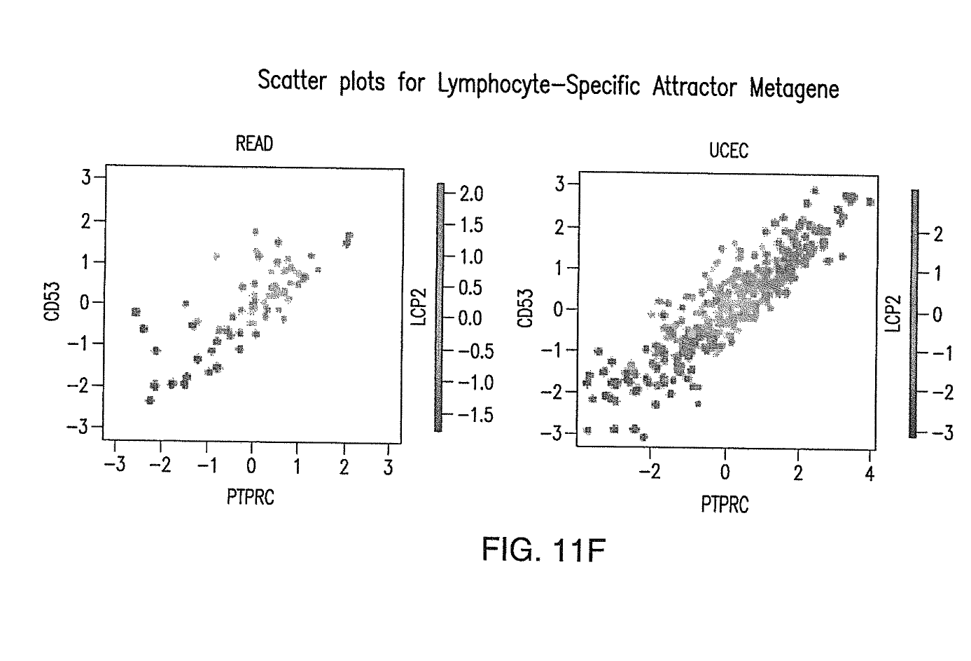

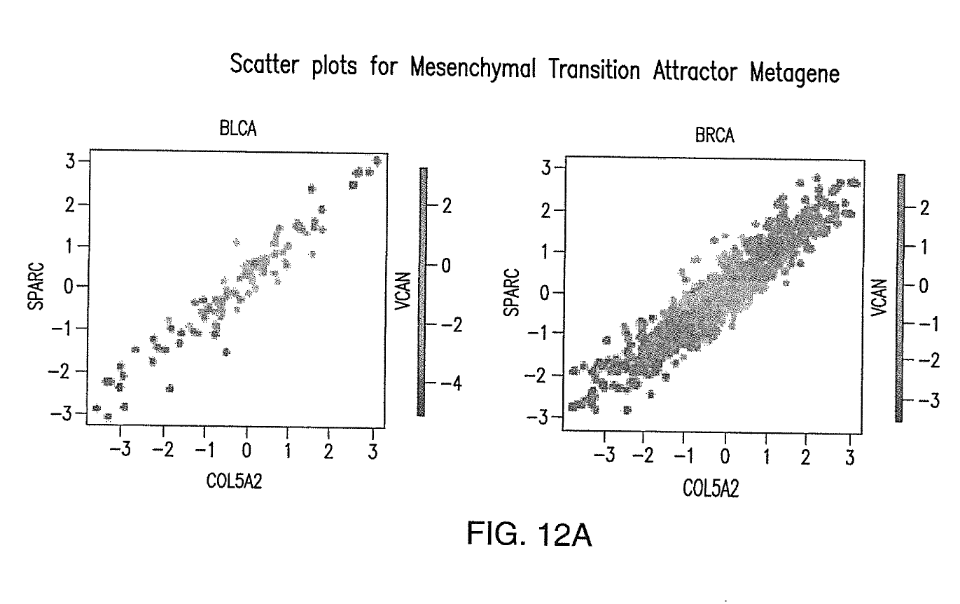

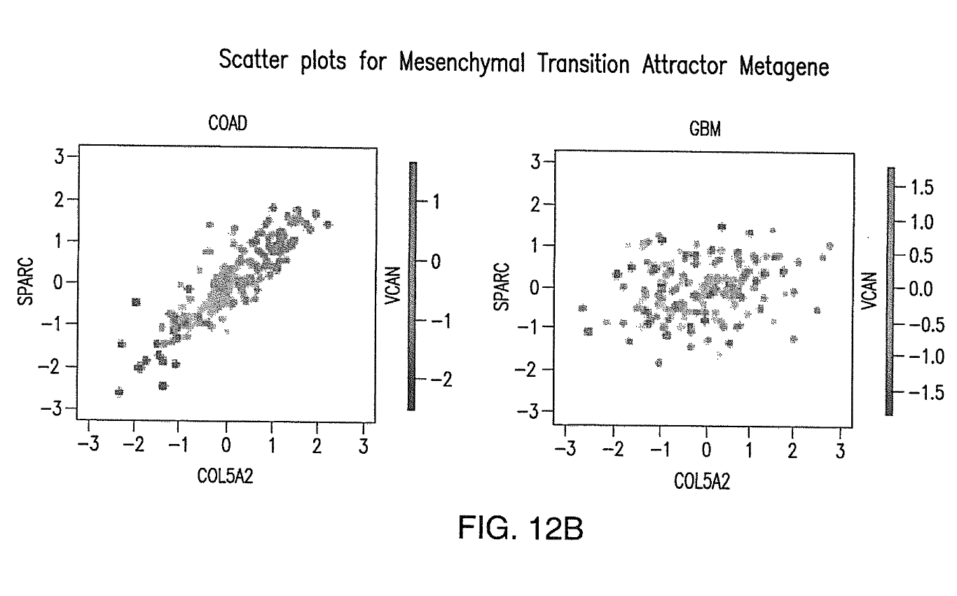

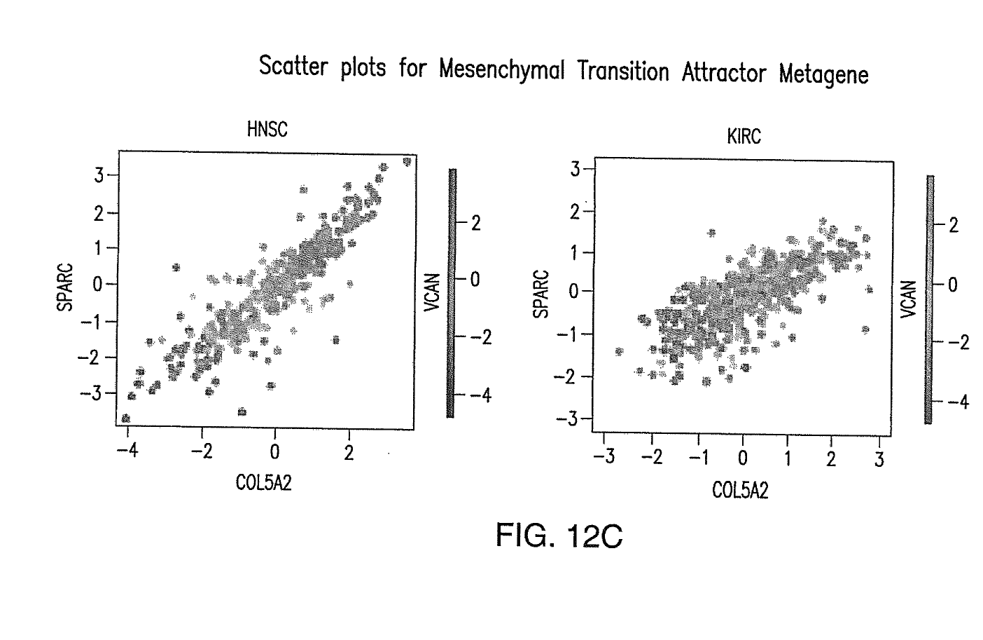

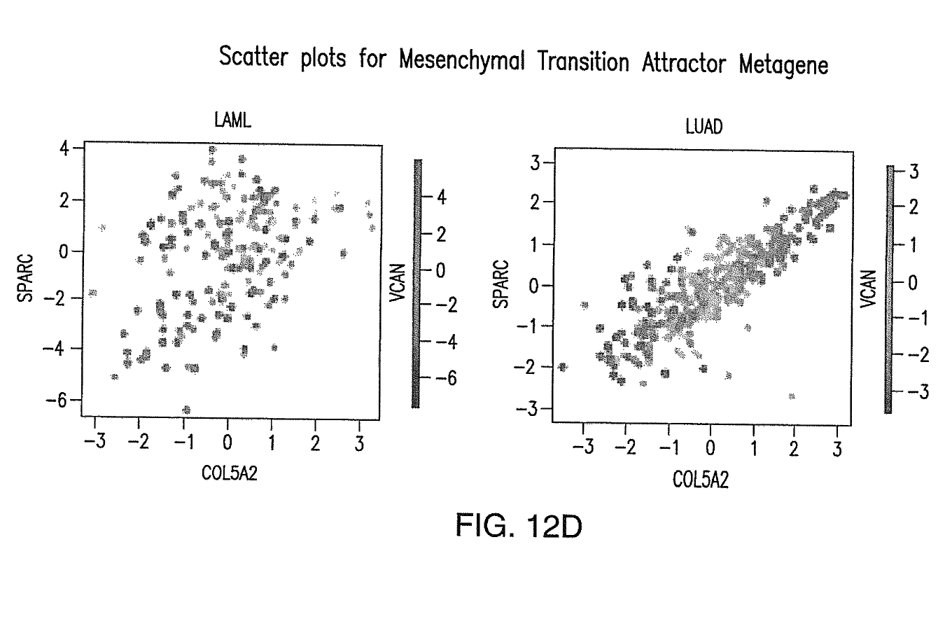

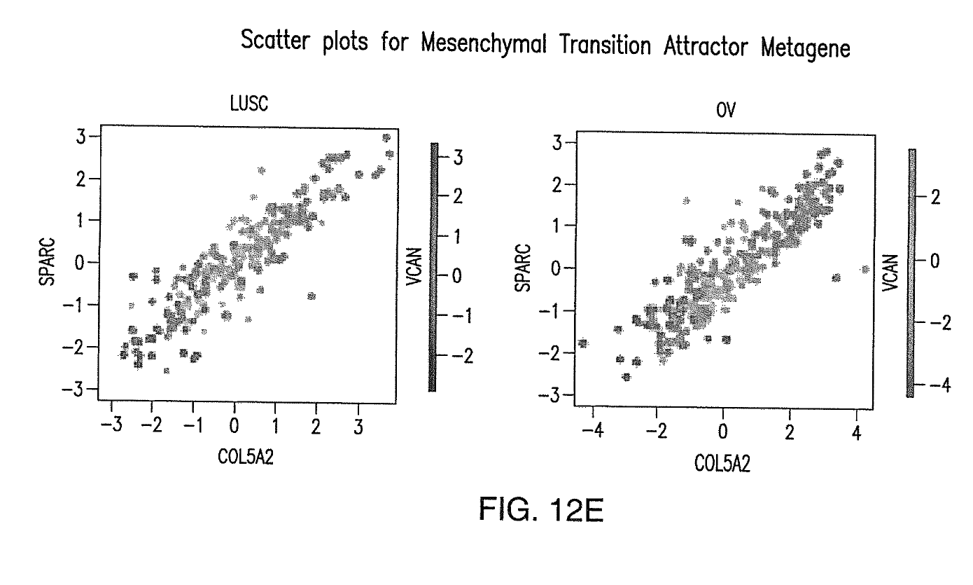

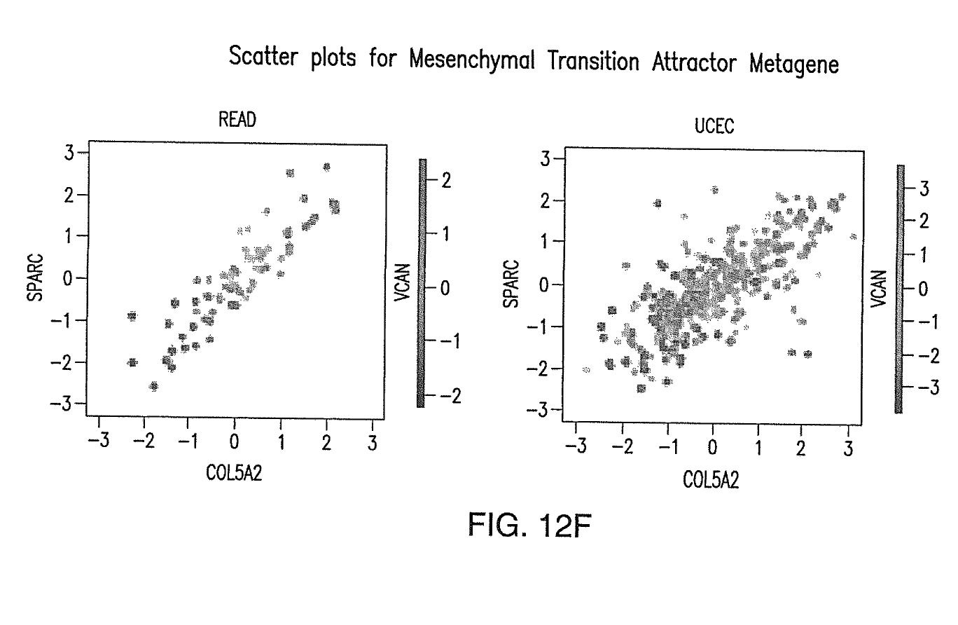

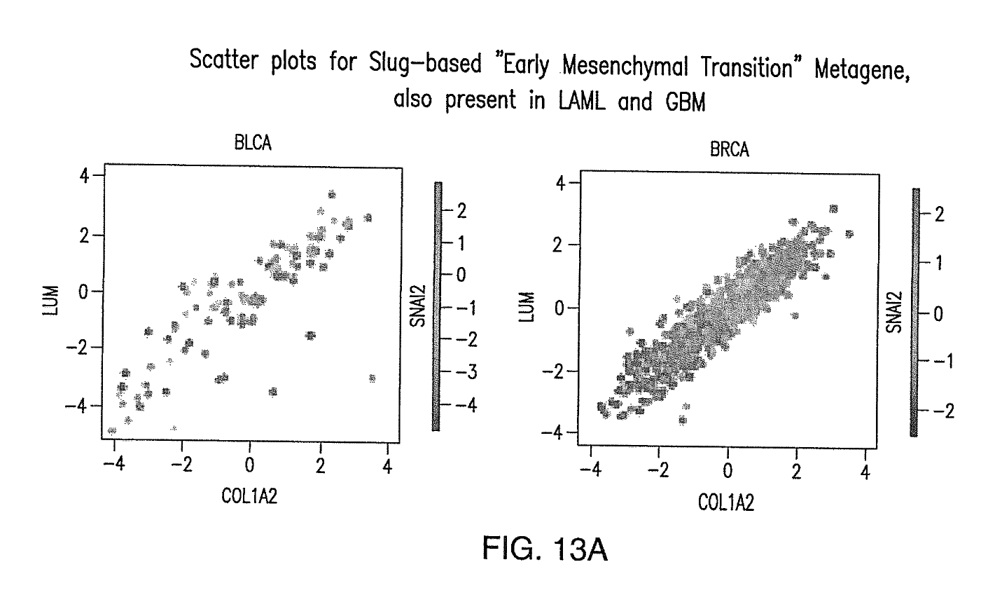

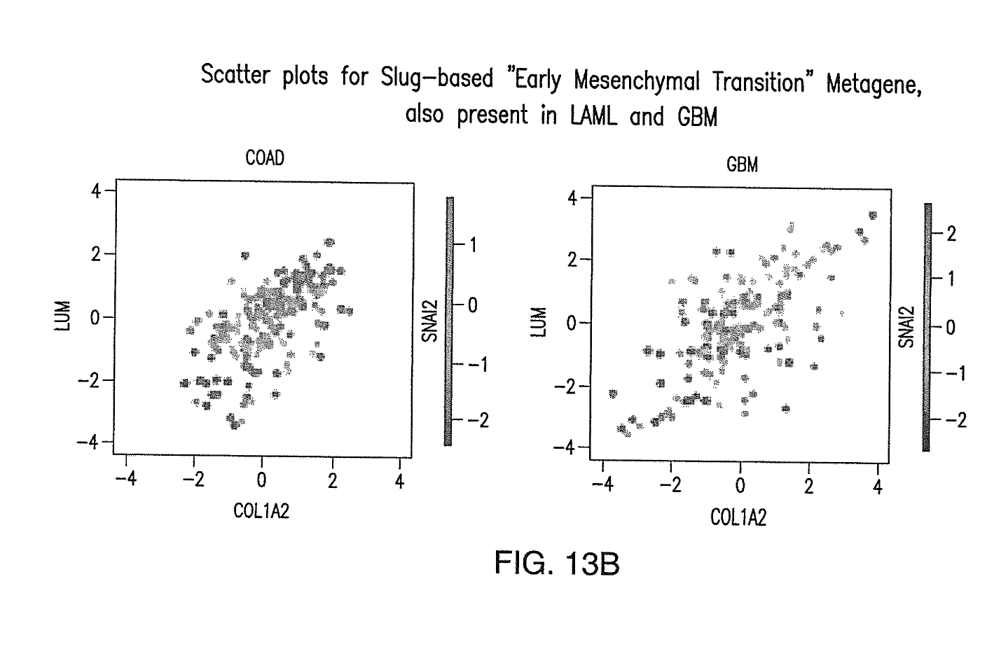

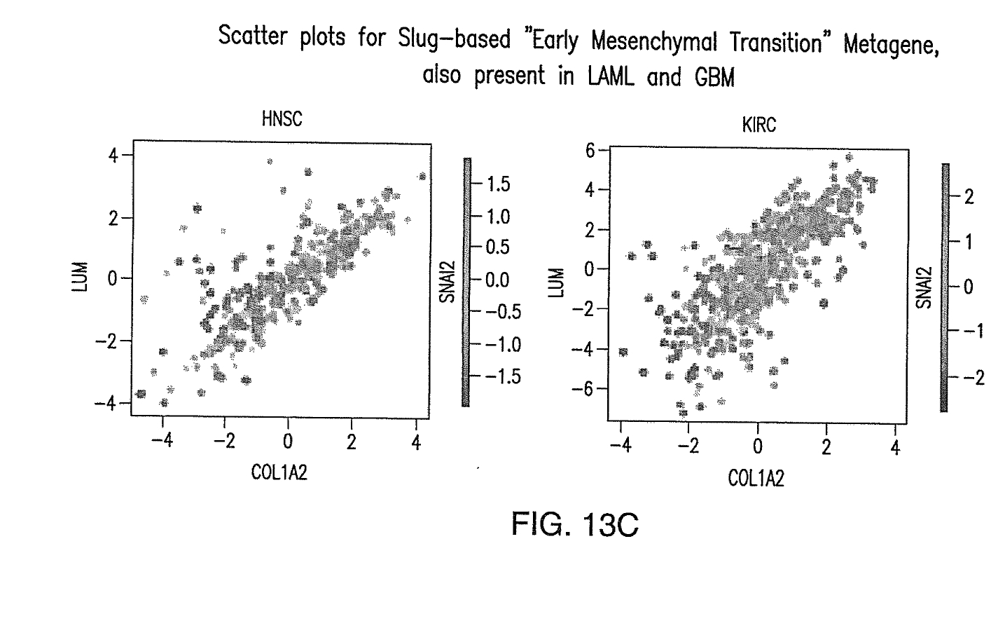

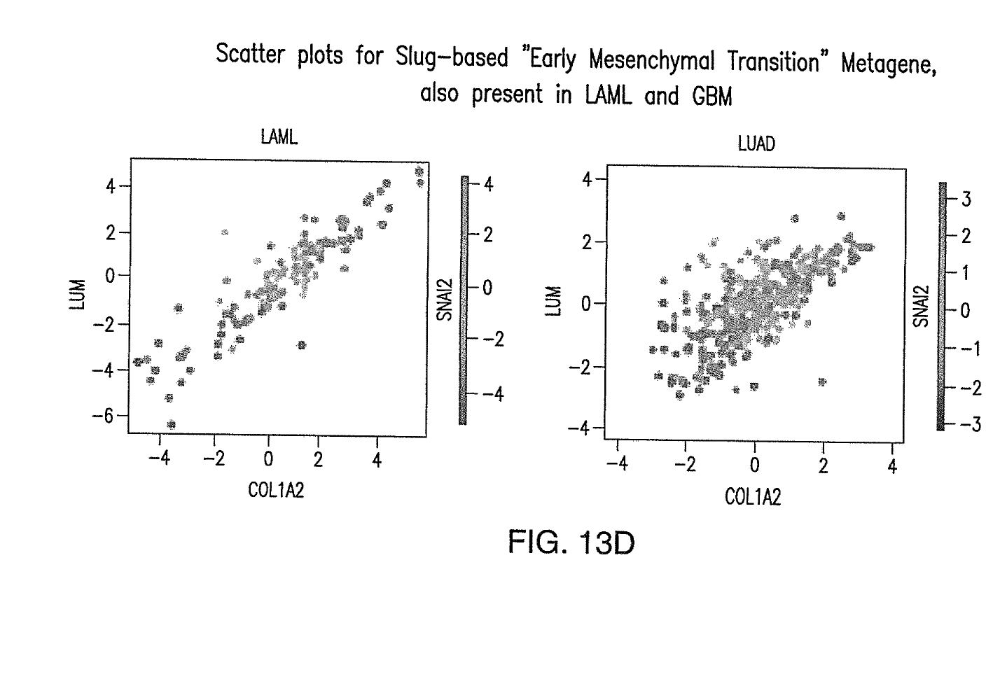

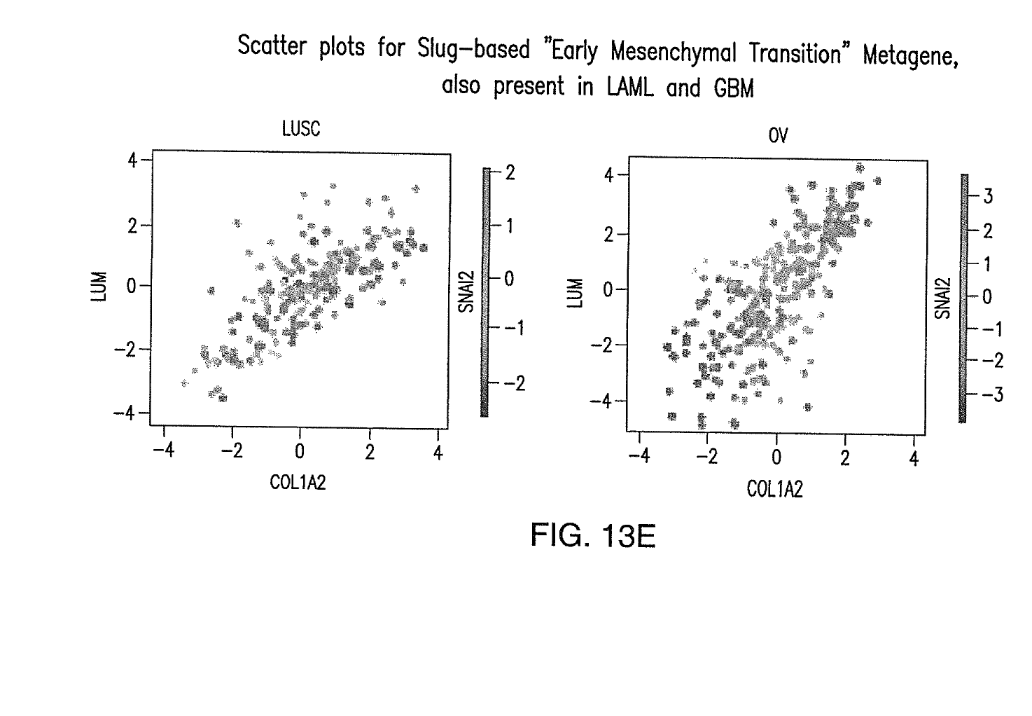

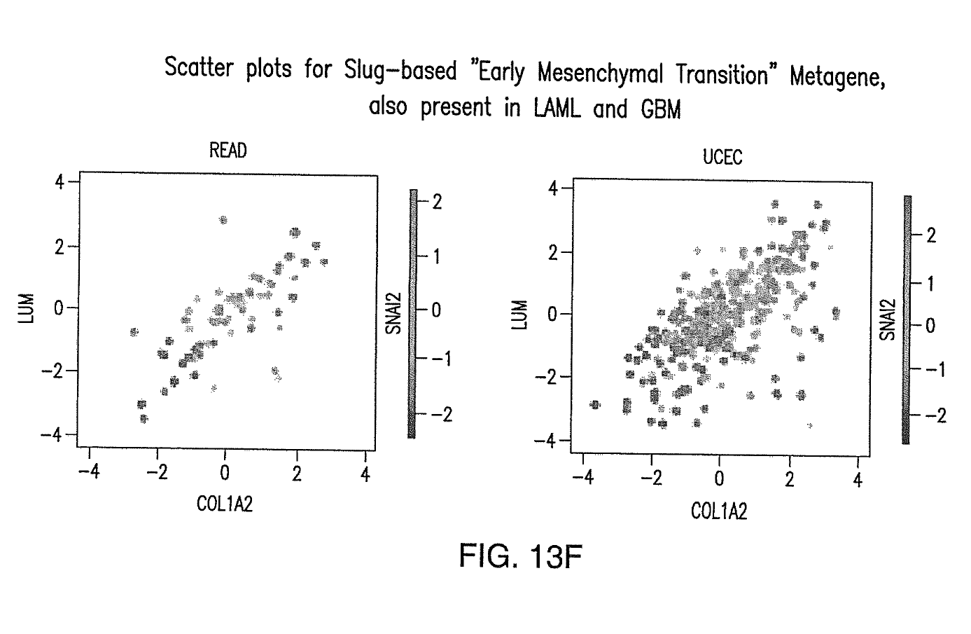

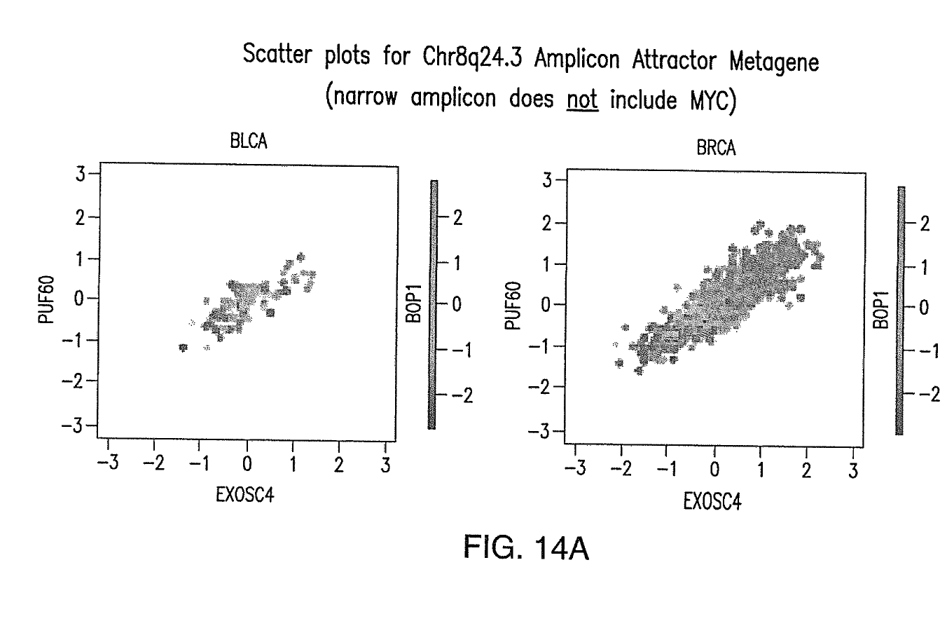

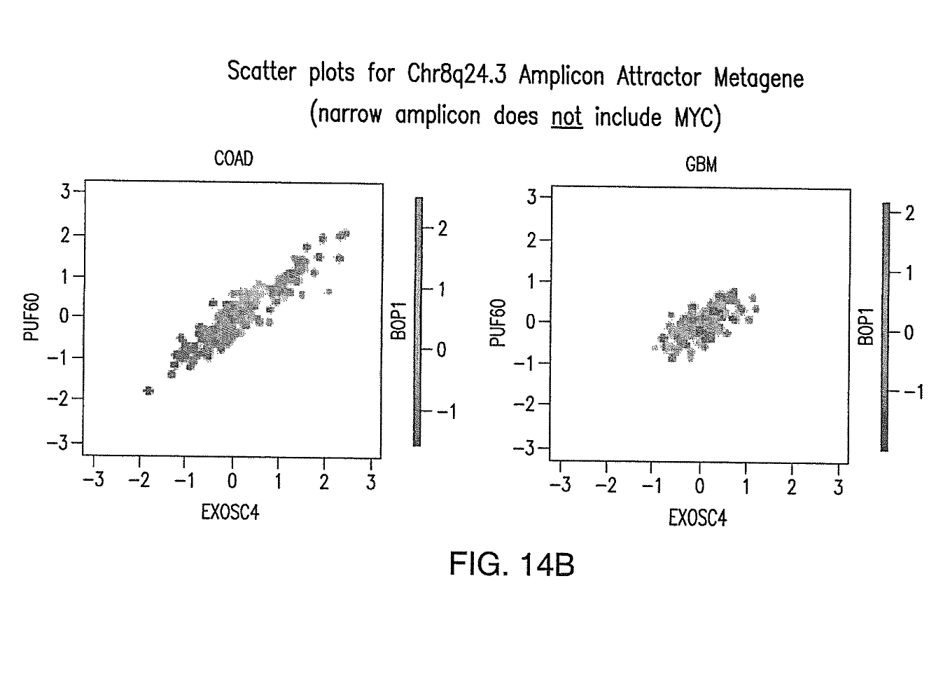

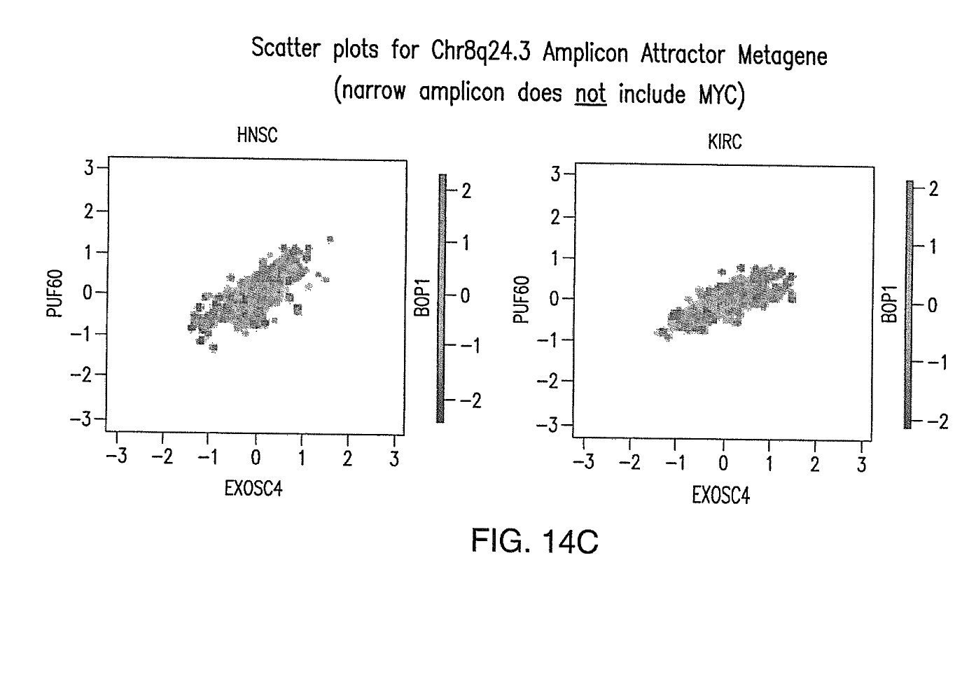

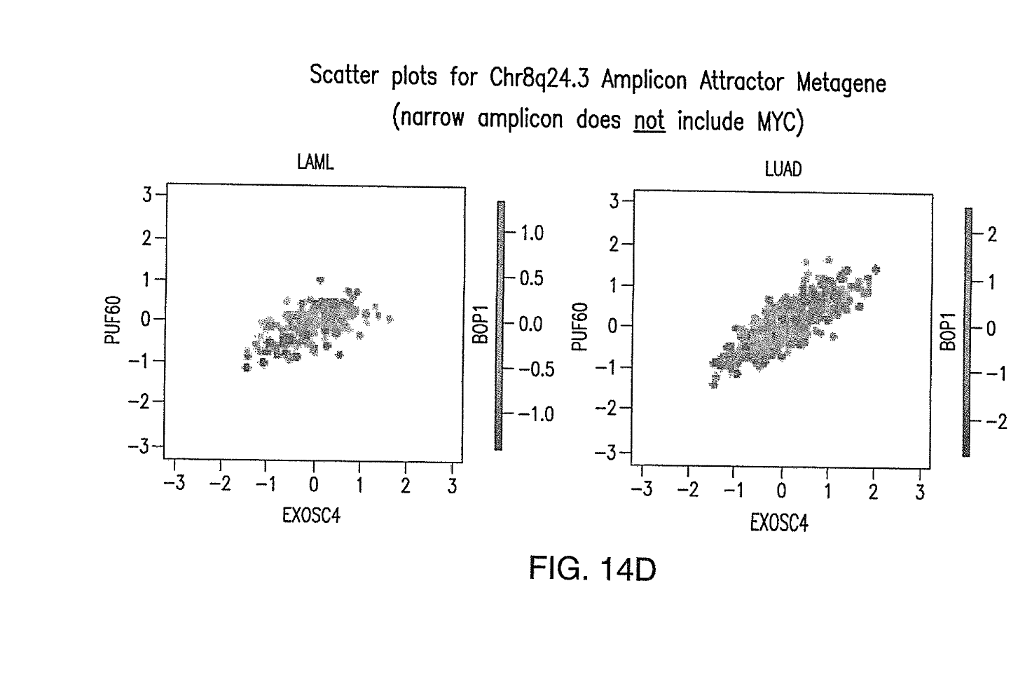

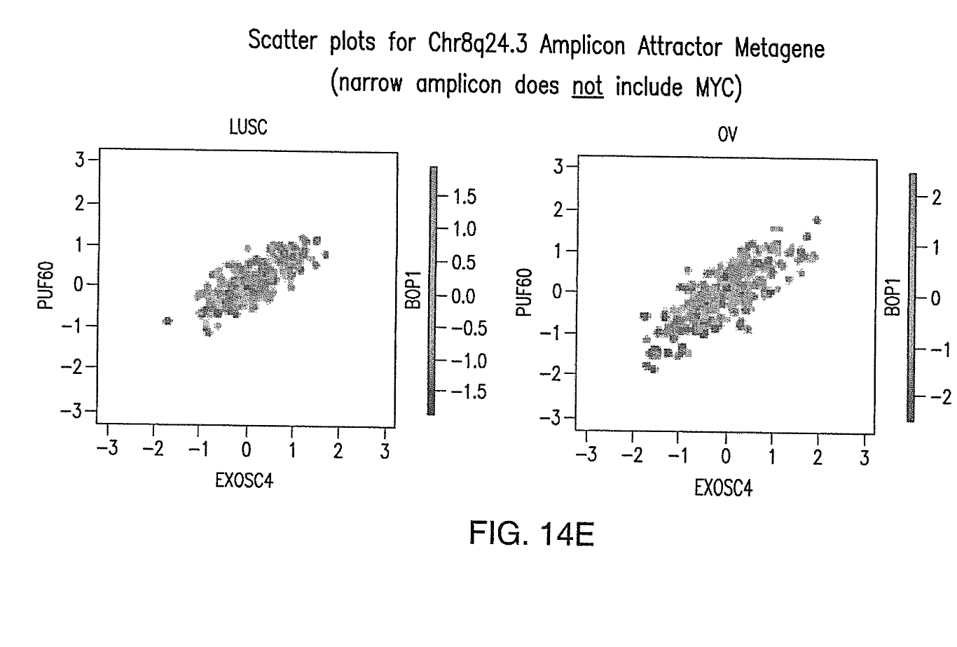

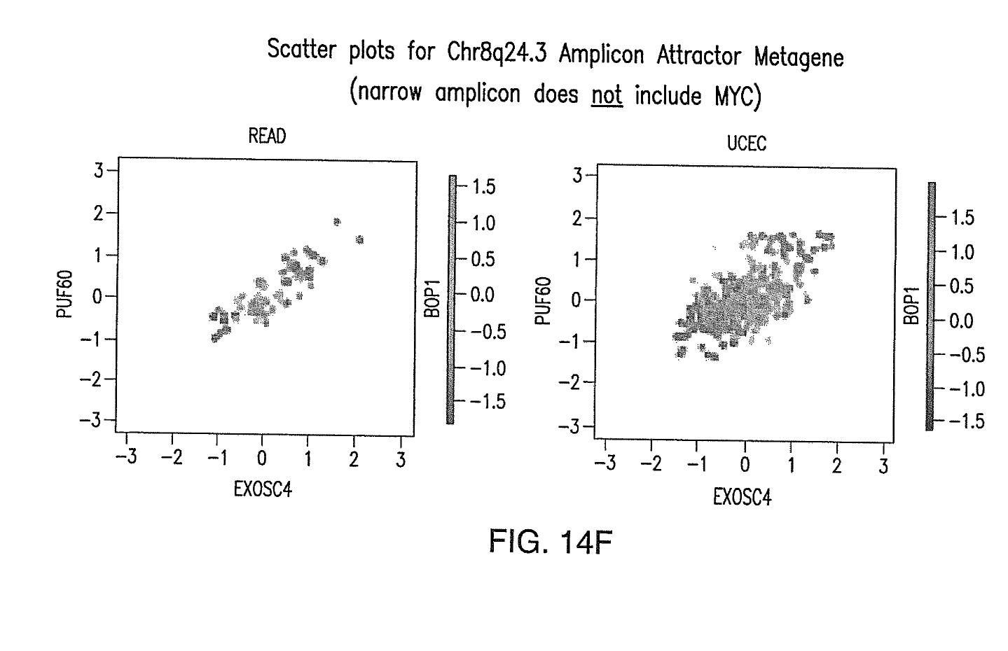

To visualize the coordinated nature of the attractor metagene expression in the various cancer types of the PANCAN12 data sets, FIGS. 10-12 depict scatter plots of the expression of the top three genes from Tables 1, 2, and 3, presented above. In virtually every case, across all three attractor metagenes, the expression of the top three genes of each attractor metagene are coordinated (coordinately less expression evidenced by dots in the bottom left corner and coordinately more expression evidenced by dots in the top right corner). In the case of the MES attractor metagene, two of the PANCAN12 datasets, two cancer types, LAML and GBM appear to lack consistent three-gene coexpression. However, when a previously-described “early version” of the mesenchymal transition metagene is employed, even the LAML and GBM cancers evidence coordinated expression (FIG. 13). Finally, similar coordinated expression is evidenced with respect to the top three genes of the Chr8q24.3 amplicon attractor metagene (FIG. 14). The coordinated expression of these attractor metagenes across the various cancer types of the PANCAN12 data sets underscores the fact that these attractor metagenes can reflect molecular mechanisms underlying different types of cancers.

Various patents, patent applications, and publications are cited herein, the contents of which are hereby incorporated by reference in their entireties.

What is claimed is:

1. A method for identifying an attractor from a data set, comprising:
evaluating the data set, wherein the data set comprises information from a plurality of objects characterized by particular feature vectors and wherein said evaluation identifies, using a computer processor, an association between individual members of plurality of objects; and
selecting, from the plurality of objects, a set of two or more objects maximally associated with a composite version of the same set of objects, and thereby identifying an attractor from said data set.

2. The method of claim 1 wherein the objects are genes, the attractor is an attractor metagene, and the composite version of the gene set comprising the attractor metagene is a weighted average of the individual genes in which the weights are proportional to the associations of the corresponding individual genes with the metagene.

3. The method of claim 2, wherein said evaluation consists of an iterative process in which each iteration modifies a metagene comprising individual genes such that the individual genes are increasingly associated with a composite version of the same set of gene.

4. The method of claim 2, wherein said evaluation consists of an iterative process in which each iteration modifies a metagene defined as a weighted average of individual genes such that the weights become increasingly proportional to the associations of the corresponding individual genes with the metagene.

5. The method of claim 2 wherein the gene data set comprises expression levels for each of the plurality of genes.

6. The method of claim 2 wherein the gene data set comprises methylation values for each of the plurality of genes.

7. A system for identifying an attractor metagene from a gene data set, comprising:
at least one processor and a computer readable medium coupled to the at least one processor, the computer readable medium having stored thereon instructions which when executed cause the processor to:
evaluate the gene data set, wherein the gene data set comprises information from a plurality of genes and wherein said evaluation identifies, using the computer processor, an association between individual members of plurality of genes; and
selecting, from the plurality of genes, a set of two or more genes maximally associated with a composite version of the same set of genes, and thereby identifying an attractor metagene from said gene data set.

8. The system of claim 7 wherein the composite version of the gene set comprising the attractor metagene is a weighted average of the individual genes in which the weights are proportional to the associations of the corresponding individual genes with the metagene.

9. The system claim 7, wherein said evaluation consists of an iterative process in which each iteration modifies a metagene comprising individual genes such that the individual genes are increasingly associated with a composite version of the same set of gene.

10. The system claim 8, wherein said evaluation consists of an iterative process in which each iteration modifies a metagene defined as a weighted average of individual genes such that the weights become increasingly proportional to the associations of the corresponding individual genes with the metagene.

11. The system of claim 7 wherein the gene data set comprises expression levels for each of the plurality of genes.

12. The system of claim 7 wherein the gene data set comprises methylation values for each of the plurality of genes.

13. A kit for detecting the presence of an attractor metagene comprising measuring means for one or more biomarker selected from the group consisting of the genes associated with an attractor metagene of FIG. 1A-B, Table 2, Table 3, or Table 4, where the measuring means is, for each biomarker to be measured, a pair of oligonucleotide primers capable of hybridizing the biomarker.

14. A method of treatment wherein a patient sample is assayed for the presence of one or more biomarker selected from the group consisting of the genes associated with an attractor metagene of FIG. 1A-B, Table 2, Table 3, or Table 4, and wherein, if said biomarker associated with the attractor metagene is present, thereafter adjusting said treatment accordingly.

15. A method of performing a prognosis of a subject wherein a patient sample is assayed for the presence of one or more biomarker selected from the group consisting of the genes associated with an attractor metagene of FIG. 1A-B, Table 2, Table 3, or Table 4, and wherein, if said biomarker associated with the attractor metagene is present, predicting the likely outcome of the cancer.
